SEQUENCE LISTING (73 sequences)

SEQ ID NO: 1 - protein, 449 aa, Homo Sapiens
QVQLQQWGAGLLKPSETLSLTCGVYGESLSGHYWSWVRQPPGKRLEWIGEIKHNGSPNYHPSLKSRVTISLDMSKNQFSLNLTSVTAADTAVYFCARRSNWPYLPFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 212 aa, Homo Sapiens
IQMTQSPSFVSASVGDRVTITCRASQGISSYLAWYQQKPGKAPKLVIYAASTLQSGVPSRFSGSGSGTEFTLTISSLQPEDFATYYCQHLIGLRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 453 aa, Homo Sapiens
QVQLQESGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 213 aa, Homo Sapiens
VLTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 5 - protein, 461 aa, Homo Sapiens
EVQLVESGGGLVKPGGSLRLSCAASGFTFSSYSMNWVRQAPGRGLEWVSSISNTSTYIYYADSVEGRFTLSRDNAKNSLYLQMNSLRAEDTAVYYCARANQHFDWLLSLLGGYHYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 218 aa, Homo Sapiens
DIVMTQSPLSLPVTPGEPASISCRSSQSLLHSNGYNYLDWYLQKPGQSPQLLIYLGSNRASGVPDRFIGSGSGTDFTLKISRVEAEDVGVFYCMQALQAVGFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 452 aa, Homo Sapiens
QVQLVQSGAEVQKPGSSVKVSCKASGGTFSTYSISWVRQAPGQGLEWMGRSIPILGIANYAQKFQGRVTFTADKSTTTAYMELSSLRSEDTAVYYCARGVGQQLVQYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 215 aa, Homo Sapiens
QSALTQPASVSGSPGQSITISCTGTSSDVGSYNLVSWYQQHPGKAPKLMIYEVSKRPSGVSNRFSGSKSGNTASLTISGLLAEDEADYYCCSYAGSSISVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 9 - protein, 449 aa, Homo Sapiens
QVQLVQSGAEVKKPGASVKVSCKASGYTFTGYYIHWVRQAPGQGLEWMGWINPNSGGTNYAQMFQGRVTMTRDTSISTAYMELSRLRSDDTAVFYCATGGSWLGGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Homo Sapiens
IVLTQSPSSLSASVGDRVTITCRASQSISSYLNWYQQKPGKAPKLLIYAASSLQSGVPSRFSGSGSGTDFTLTISSLQPDDFATYYCQRSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, Homo Sapiens
EVQLVESGGGLVQPGGSLRLSCAASGFTFSNNAMSWVRQTPGKGLEWVSSFSGGRDTTYYADSVKGRFTISRDDSKNTLFLQMSSLRAEDTAVYYCAKDLGLLRGIANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Homo Sapiens
QSALTQPASVSGSPGQSITISCTGTSSDVGTYNSVSWYQQHPGKAPNLIIYDVTNRPSGVSNRFSGSKSGNTASLTISGLQTEDEADYYCSSYRRTNTLGFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 13 - protein, 452 aa, Homo Sapiens
QVQLVQSGAEVKKPGASVKLSCKASGYTFNSYYINWLRQAPGQGLEWMGIINPSSSSTNYAQNFQGRVTMTRDTSTSTVYMELSSLRSEDTAVYYCARNYAGIEARGWLDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 211 aa, Homo Sapiens
EIVLTQSPGTLSLSPGERATLSCRASQSVSSRSLAWYQQKPGQAPRLLIYGASSRATGIPDRFSGSGSGTDFTLTISRLEPEDFAVYYCQQSTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 453 aa, Homo Sapiens
QVQLVQSGAEVKKPGASVKVSCKASEYSFTGYYLHWVRQAPGQGLEWMGWINPNNGDTRSAQRFQGRVTMTRDTSISTAYMEVSSLTSDDAAIYYCARARWDLLPGGRCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 215 aa, Homo Sapiens
QSALTQPRSVSGSPGQSVTISCTGTSSDVGSYTYVSWYQQHPGKAPKLIVSDVSERPSGVPDRFSGSKSGNTASLTISGLQAEDEADYYCSSYAGSYTFVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 17 - protein, 450 aa, Homo Sapiens
QVQLQESGAGLLKPSETLSLTCAVYGGSFSGYYWSWIRQPPGKGLEWIGEIIHSGSTNYNPSLKSRVTISVDTSNNQFSLKLSSVTAADTAVYYCARGRRLLWFGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, Homo Sapiens
EIVLTQSPGTLSLSPGERATLSCRASQSVSGSYLAWYQQKPGQAPRLLIYGASSRATGIPDRFNGSGSGTDFTLTISRLEPEDFAVYYCQQYGSSPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 450 aa, Homo Sapiens
QVQLVQSGAEVKKPGASVKVSCKASGYTFTGYYMHWVRQAPGQGLEWMGGINSNSGGTNFAQKFQGRVTMTRDTSISTAYMELSRLRSDDTAVYYCASTYSSTWFRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 216 aa, Homo Sapiens
EIVLTQSPGTLSLSPGERATLSCRASQSISINYLAWYQQRPGQAPRLLISGASSRATGIPDRFSGSGSGTDFTLTISRLEPEDFAVYYCQQYGSSPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 453 aa, Homo Sapiens
QVQLVQSGAEVKKPGASVKVSCKASGYTFIGYYMHWVRQAPGQGLEWMGWINPNSGGTNYAQKFQGRVTMTRDTSIRTVYMELSRLRFDDTAMYYCARAPSLVVGGGRLVDYWGQGSQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 215 aa, Homo Sapiens
QSALTQPRSVSGSPGQSVTISCTGTSSDVGGYNYVSWCQQHPGKAPQLMIYDVSKRPSGVPDRFSGSKSGNMASLTISGLQAEDEGDYYCCSYAGNYTLVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 23 - protein, 449 aa, Homo Sapiens
QVQLVQSGPEVKKPGTSMKISCKASGFTFTRSTMQWVRQARGQRLEWIGWIVVGSGNTNYAQKFQERVTITRDMSTSTAYMELSSLRSEDTAVYYCAAAPVGPTSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 212 aa, Homo Sapiens
DIQMTQSPSSLSASVGDRVTITCRASQSIINYLNWYQQKPGRAPKLLIYAASSLLSGVPSRFSGSGSGTDFTLTISSLQPEDFATYYCQQSYSTPYTFGQGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 25 - protein, 452 aa, Homo Sapiens
QVQLVQSGAEVKKPGASVKVSCKASGYIFISYFMHWVRQAPGQGLEWMGIINPSSGDTRYAQKFQGRVTMTRDTSTNTVYMELSSLRSDDTAVYYCARRPGGLERHNWLDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 215 aa, Homo Sapiens
EIVMTQSPATLSVSPGERATLSCRASQSISSNLAWYQQKPGQAPRLLIYGASTRATGTPARFSGSGSGTEFTLTISSLQSEDFASYYCQQYNNWPAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 449 aa, Homo Sapiens
QVQLVQSGAEVKKPGASVKVSCKASGYTFTGYYMHWVRQAPGQGLEWMGRINPNTGGTNYAQKFQGRVIMTRDTSIKTTYMELSSLRSDDMAVYYCARSATGYYGMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, Homo Sapiens
DIQMTQSPSSLSASVGDRVTITCRASQSIIKYLNWYQQRPGKAPKLLIYATSTLQSGVPARFSGSGSGTDFTLTISSLQPEDFATYYCQQSYSTLWTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 452 aa, Homo Sapiens
QVQLVQSGAEVKKPGASVKVSCKASGYTFIAYYMHWVRQAPGQGLEWMGWINPNSGGTNYAQKFQGRVTMTRDTSISTAYMELSRLRFDDTAVYYCGRFSGNYFLYHGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 219 aa, Homo Sapiens
DIVMTQSPDSLALSLGERATINCKSSQSVLYNSNNKNYLAWYQQKPGQPPKLLIYWASTRESGVPDRFSGSGSGTDFTLTISSLQAEDVAVYYCQQYYSNLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 456 aa, Homo Sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYFCARVWVYYYDSSGYSYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, Homo Sapiens
EIVLTQSPATLSLSPGERATLSCRASQSVSSYLAWYQQKPGQAPRLLIYDASKRATGIPARFSVSGSGTDFTLTISSLEPEDFAVYYCQQRSHWTWTFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 449 aa, Homo Sapiens
QVQLVQSGAEVKKPGASVKVSCKASGYTFIGYYIHWVRQAPGQGLEWMGWINPNSGGTNYAQKFQGRVTMTRDTSITTAYMELTRLRSDDTAVYYCARGGLPGTGTAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 215 aa, Homo Sapiens
DIQLTQSPSSLSASVGDRVTITCRASQGISNYLAWYQQKPGKIPRLLIYAASTLQSGVPSRFSGSGSGTDFTLTISSLQPEDVATYYCQKFNSVPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 456 aa, Homo Sapiens
QVQLVQSGAEVKKPGASVKVSCKASGYTFTSYDINWVRQATGQGLECMGWMNPNSGNTGYAQKFQGRVTMTRNTSISTAYMELSSLRSDDTAVYYCARGLRTYYYGSGYYRPLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, Homo Sapiens
DIQMTQSPSSLSASVGDRVTITCRASQSISNYLNWYQQKPRKAPKLLIYAASSLQSGVPSRFSGSGSGADFTLTISSLQPEDFATYYCQQSYSTLQTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 449 aa, Homo Sapiens
EVQLVESGAEVKKPGASVKVSCKASGYTFTGYYMHWVRQAPGQGLEWMGWINPNSGGTNYAPKFQGRVTMTRDTSISTAYMELTGLRSDDTAVYYCARSITLIVNFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, Homo Sapiens
DIVLTQSPSSLSASVGDRVTITCRTSQSISNYLNWYQQKPGKAPKLLIYAASSLQSGVPSRFSGSGSGTDFTLTVSSLQPEDFATYYCQQTYNTPVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 451 aa, Homo Sapiens
QVQLVQSGAEVKKPGASVKVSCKASGYTFISYYMHWVRQAPGQGLEWMGIINSSGGSTNYAQKFQGRVTMTRDTSTSTVYMELSSLRSEDTAVYYCARGGITLVRGVIYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Homo Sapiens
EIVLTQSPSSLSASVGDRVTITCRASQGISNSLAWYQQKPGKAPKLLLYAASRLESGVPSRFSGSGSGTDYTLTISSLQPEDFATYYCQQYYSTLPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 453 aa, Homo Sapiens
QVQLQESGPGLVKPSQTLSLTCTVSGGSISSGGYYWSWIRQHPGKGLEWIGNIYYSGSTYYNPSLKSRITISLDTSKNQFSLKLSSVTAADTAVYYCARTSRTTVLRNAFDIWGHGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 216 aa, Homo Sapiens
QSALTQPRSVSGSPGQSVTISCTGTSSDVGGYDYVSWYQQHPGKAPKLMISDVSKRPSGVPDRFSGSKSGNTASLTISGLQPEDEADYYCCSYAGTYTYWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 43 - protein, 456 aa, Homo Sapiens
QVQLVQSGAEVKKPGASVKVSCKASGYTFTGYYIHWVRQAPGQGLEWMGWINPNSGGTNYAQKFQGRVTMTRDTSISAAYMELSSLLFDDTAVYYCARVPHIVVVVAAANANFDYWGQGTLVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 214 aa, Homo Sapiens
DIQMTQSPSSVSASVGDRVTITCRASQGISTWLAWYQQKPGKAPKALIYAATRLQSGVPSRFSGSGSGTDFTLTISSLQPEDFAIYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 459 aa, Homo Sapiens
VQLVESGAEVKKPGASVQVSCKASGYIFKSYYVHWVRQAPGQGLEWMGIINPSGGTASYAQKFQGRVTMTRDTSTSTVYMELSSLRSEDTAVYYCARDNGIVGYSGSRGYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 214 aa, Homo Sapiens
EIVLTQSPSSLSASVGDRVTFTCRASQSMSSCLNWYQQKPGKAPKLLIYAASSLQSGVPSRFSGSGSGTDFTLTISSLQPEDFATYYCQQSYSTPYTFGQGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 453 aa, Homo Sapiens
QVQLVQSGAEVQKPGASVKVSCKASGYTFASYDINWVRQATGQGLEWMGWMNPKTGNTGYAQKFQGRVTLTRNTSISTAYMELTSLRSEDTAVYYCATYRIIAAVGYRYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, Homo Sapiens
DIQLTQSPSSLSASVGDSVTITCRASQGFGNYLAWYQQRPGKVPEVLIYAATTLQSGVPSRFSGSGSGTDFTLTISSLQPEDVATYYCQKYNSAPFTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 456 aa, Homo Sapiens
EVQLVESGPGLVKPLETLSLTCAVPGGSIRRNYWSWIRQPPGKGLEWIGHSYGSGGSTNYNPSLESRVTLSVDTSKNLFSLKLTSVTAADTAVYYCARTVWYYTSGTHYFDHWGQGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCPAPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSVSPGK

SEQ ID NO: 50 - protein, 215 aa, Homo Sapiens
QSVLTQPPSVSAAPGQKVTISCSGSSSNIGRSYVSWYQQVPGAAPKLLIYDTNKRPSGVSDRFSGSKSGSSASLAITGLQTGDEADYYCGAWDGSLNVHIFGSGTKLTVLGQPKASPLVTLFPPSSEELQANKATLVCLISDFYPGVVKVAWKADGNSVNTGVETTTPSKQSNNKYAASSYLSLTSDQWKSHKSYSCQVTHEGSTVEKTVAPTEC

SEQ ID NO: 51 - protein, 448 aa, Homo Sapiens
QVQLVQSGAEVKKPGSSVRVSCKASGGSFSRYTVNWVRQAPGQGLEWMARFIPIFNMPDYAPKFQGRITITADESTSTAYLELSSLTSDDTAVYYCASRQHHEYFQEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 215 aa, Homo Sapiens
EIVLTQSPGTLSMSPGERATLSCRASRTVSSSNLAWYQQKPGQAPRLLIYDVSSRATGIPDRFSGRGSGTDFTLTISRLEPEDFAVYYCQQYGTSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 449 aa, Homo Sapiens
EVQLVESGGGLVQPGGSLRLSCAASGFTFSTYAMSWVRQAPGKGLEWVSSINNSGRNTFSADSVKGRFTISRDNSKNTLFLVMNSLRAEDTAVYYCAKDLRLGGGSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 216 aa, Homo Sapiens
QSALTQPASVSGSPGQSITISCTGTSSDVGSYNFVSWYQQHPGKAPKLMIYEVSERPSGISNRFSGSKSGNTASLTISGLQAEDEADYYCSSYAGSTTFRVFGGGTKLTVRGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 55 - protein, 457 aa, Homo Sapiens
TGVHSEVQLVESGPGLVKPSQTLSLTCTVSGASISSGGYFWSWIRQHPGKGLEWIGNIYYIGNTYYNPSLKSRLTISVDTTQNQFSLKLTSVTAADTAVYYCARVPRLRGGNYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, Homo Sapiens
QSVLTQPASVSGSPGQSITISCTGTSSDVGGYKYVSWYQQHPDKAPKLMIYEVSNRPSGVSNRFSGSKSGNTASLTISGLQAEDEADYYCSSYTSSSTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTE

SEQ ID NO: 57 - protein, 458 aa, Homo Sapiens
QVQLLESGAEVKKPGSSVKVSCKASGDTFIRYSFTWVRQAPGQGLEWMGRIITILDVAHYAPHLQGRVTITADKSTSTVYLELRNLRSDDTAVYFCAGVYEGEADEGEYDNNGFLKHWGQGTLVTVTSASTKGPSVFPLAPSSKSTSGGTAALGCLVKDYFPQPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 216 aa, Homo Sapiens
ELELTQSPATLSVSPGERATLSCRASESVSSDLAWYQQKPGQAPRLLIYGASTRATGVPARFSGSGSGAEFTLTISSLQSEDFAVYYCQQYNNWPPRYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 460 aa, Homo Sapiens
EVQLVQSGAEVKKPGSSVKVSCKASGGTFSNYAINWVRQAPGQGLEWMGGIIPIFNIAHYAQRFQGRVSITADESTSTAYMELSSLRSEDTAVFYCASPYPNDYNDYAPEEGMSWYFDLWGRGTLVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 214 aa, Homo Sapiens
DIQMTQSPSTLSASVGDRVTITCRASQSISNWLAWYQQKPGRAPKLLMYKASSLKSGVPSRFSGSGSGTEFTLTISSLQSDDFATYYCQQHDSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 61 - protein, 449 aa, Homo Sapiens
EVQLVQSGAEVKKPGATVKISCKASGYTFSDFYMYWVRQAPGKGLEWMGLIDPEDADTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 213 aa, Homo Sapiens
DIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 63 - protein, 464 aa, Homo Sapiens
EVQLVQSGAEVNKPGSSVKVSCQASGATLNSHAFSWVRQAPGQGLEWMAGIIPIFGSSHYAQKFRGRVTISADESTRTVYLHLRGLRSDDTAVYYCASNSIAGVAAAGDYADYDGGYYYDMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 215 aa, Homo Sapiens
QSILTQPPSVSAAPGQKVTISCSGSSSNIGNNDVSWYQQFPGTVPKLVIYENNERPSGIPDRFSGSKSGTSATLGITGLQTGDEADYYCGTWDSSLSAVVFGGGSKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 65 - protein, 462 aa, Homo Sapiens
QVQLVQSGAEVKKPGSSVKVSCRASRGTFSSYGITWVRQAPGQGLEWMGGIIPIFDVTNYAQNFQGRVAITTDAEMSTAYMELRSLKSEDSAVYYCASDSRDFSYYEPGTSYSHYYNIMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 215 aa, Homo Sapiens
EIVMMQSPGTLSLSPGERATLSCRASQSVSSSYLAWYQQKPGQAPRLLIYGASSRATGIPDRFSGSGSGTDFTLTISRLEPEDFAVYYCQQYGSSPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 462 aa, Homo Sapiens
EVQLLEQSGAEVKKPGSSVQVSCKASGGTFSMYGFNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 215 aa, Homo Sapiens
ELVLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTVAAPSVFIFPPSDEQLKSGTASVVCLLNNFYPREAKVQWKVDNALQSGNSQESVTEQDSRDSTYSLGSTLTLSKADYEKHKVYACEVTHQGLSSPVTKSFNRGEC

SEQ ID NO: 69 - protein, 185 aa, Homo Sapiens [sequence omitted]

SEQ ID NO: 70 - protein, 26 aa, Artificial sequqnce
  Modified residuals at positions 1, 20, and 22
  MOD_RES (1)..(1): thiopropionyl is attached to Asparagine
  MOD_RES (20)..(20): D-proline
  MOD_RES (22)..(22): p-cyclohexylmethoxy is attached to phenylalanine
NLHFCQLRCKSLGLLGRCAPTFCACV

SEQ ID NO: 71 - protein, 80 aa, Artificial sequqnce
  Linker sequence. Repeat of (GGGGX)n, wherein n=2-16; X is serine or threonine.
  misc_feature (5)..(5): Xaa can be any naturally occurring amino acid
  REPEAT (6)..(80): The sequence is (GGGGX)n, wherein n=2-16; X is serine or threonine. Therefore, the minimal sequence is GGGGXGGGGX, wherein n=2.  Any number of further repeats from the third to sixteenth can be absent.
GGGGXGGGGXGGGGXGGGGXGGGGXGGGGXGGGGXGGGGXGGGGXGGGGXGGGGXGGGGXGGGGXGGGGXGGGGXGGGGX

SEQ ID NO: 72 - protein, 40 aa, Artificial sequqnce
  Linker sequence
GGGGSGGGGSGGGGSGGGGSGGGGSGGGGSGGGGTGGGGT

SEQ ID NO: 73 - protein, 40 aa, Artificial sequqnce
  Linker sequence
GGGGSGGGGSGGGGSGGGGSGGGGSGGGGSGGGGSGGGGS

(19) United States
(12) Patent Application Publication
(10) Pub. No.: US 2024/0285790 A1
(43) Pub. Date: Aug. 29, 2024

(54) ANTIBODY-CD4 CONJUGATES AND METHODS OF USING THE SAME

(71) Applicants: THE HENRY M. JACKSON FOUNDATION FOR THE ADVANCEMENT OF MILITARY MEDICINE, INC., Bethesda, MD (US); CENTRE HOSPITALIER DE L'UNIVERSITE DE MONTREAL, Montreal, Quebec (CA)

(72) Inventors: Marzena PAZGIER, Derwood, MD (US); William D. TOLBERT, North Bethesda, MD (US); Dung N. NGUYEN, Beltsville, MD (US); Andres FINZI, Quebec (CA); Jonathan RICHARD, Quebec (CA)

(21) Appl. No.: 18/565,379

(22) PCT Filed: Jun. 10, 2022
(86) PCT No.: PCT/US2022/032958

§ 371 Date: Nov. 29, 2023

Related U.S. Application Data
(60) Provisional application No. 63/209,192, filed on Jun. 10, 2021.

(51) Int. Cl. A61K 47/68; A61K 45/06; A61P 31/18

(52) U.S. Cl. CPC A61K 47/6841; A61K 45/06; A61K 47/6811; A61K 47/6889; A61P 31/18

(57) ABSTRACT

The present disclosure provides for antibody conjugate molecules (Ab-CD4 conjugates) comprising an antibody, at least one linker, and at least one CD4 compound, wherein the at least one linker links the antibody to the at least one CD4 compound and wherein the Ab-CD4 conjugate is capable of neutralizing an HIV virus, as well as methods of killing HIV-infected cells through an Fc-mediated effector function using the Ab-CD4 conjugates. Also provided are pharmaceutical compositions comprising the Ab-CD4 conjugates and methods of treating or preventing HIV injection in a subject.

### CROSS-REFERENCE TO RELATED APPLICATION

This application claims the benefit of, and relies on the filing date of, U.S. provisional patent application No. 63/209,192, filed 10 Jun. 2021, the entire disclosure of which is incorporated herein by reference.

### GOVERNMENT INTEREST

This invention was made with government support under R01AI129769 awarded by the National Institutes of Health. The government has certain rights in the invention.

### FIELD

This application relates generally to antibody conjugates comprising a human CD4 or CD4 mimetic compound (CD4mc), which may include small CD4 mimetic compounds or peptide-based mimetic compounds, linked or conjugated to an antibody specific for the constant region 1 and 2 (C1C2) (referred to as Cluster A epitope region) or the co-receptor binding site (CoRBS) epitope region of the HIV envelope glycoprotein (Env) and capable of neutralizing HIV virions and sensitizing and killing HIV-infected cells through Fc-mediated effector functions, including antibody-dependent cellular cytotoxicity (ADCC).

### BACKGROUND

An estimated 1.7 million new cases of human immunodeficiency virus (HIV) infection were diagnosed worldwide in 2019, and approximately 38.0 million people are currently living with AIDS/HIV. Although AIDS-related deaths have been dramatically reduced in recent years, an estimated 690,000 people nonetheless died from AIDS-related complications worldwide in 2019, and there remains no cure.

HIV is a retrovirus that infects CD4+ cells of the immune system, destroying or impairing their function. As the infection progresses, the immune system becomes weaker, leaving the infected person more susceptible to opportunistic infections and tumors, such as Kaposi's sarcoma, cervical cancer, lymphoma, and neurological disorders. The most advanced stage of HIV infection is acquired immunodeficiency syndrome (AIDS). It can take 10-15 years for an HIV-infected person to develop AIDS, and certain antiretroviral drugs can delay the process even further.

Although much effort has been put forth into designing effective therapeutics against HIV, currently no curative anti-retroviral drugs against HIV exist. Nonetheless, several stages of the HIV life cycle have been evaluated as potential targets for the development of therapeutic agents, including targeting HIV's ability to replicate after infection in a cell and targeting HIV's ability to enter into a cell.

With respect to HIV's ability to enter into a cell, the envelope glycoprotein trimer (Env) is known to play a role in HIV virus attachment and subsequent entry into host cells. The mature Env trimer is comprised of non-covalently associated gp120-gp41 heterodimers that are formed by furin cleavage of a gp160 precursor [1]. To initiate the viral entry process, the outer gp120 protomer of the Env trimer binds to a receptor CD4 on the host cell surface. After binding to CD4, the Env trimer undergoes conformational changes that lead to the formation of a co-receptor binding site (CoRBS) and engagement of CCR5 and/or CXCR4, two known HIV-1 co-receptors [2-9]. Additional structural rearrangements within the Env trimer lead to the formation of a six-helix bundle from the helical heptad repeat HR1 and HR2 segments of the gp41 ectodomain in order to drive fusion of the viral and target cell membranes [10, 11].

The Env trimer is the only viral protein present on the surface of virions and HIV-1 infected cells; therefore, it represents a major antibody-targeted HIV-1 antigen. Env presentation to a host immune system elicits antibody responses against many diverse Env sites. These antibodies can impact HIV-1 through various mechanisms, including direct virus neutralization and Fc-effector activities, including antibody-dependent cellular cytotoxicity (ADCC) and/or antibody-dependent cellular phagocytosis (ADCP) of infected cells. In HIV-1 infection, a number of elicited antibodies target epitopes that are occluded and, therefore, inaccessible or poorly accessible, in the unliganded Env trimer. These antibodies usually lack direct neutralization activity and therefore are referred to as non-neutralizing antibodies [12, 13]. Some Env epitopes recognized by non-neutralizing antibodies map to highly-conserved regions of the Env trimer and have therefore been deemed potential targets for protective humoral responses and antibody therapeutics. This potential, however, is impeded by these epitopes' lack of exposure or accessibility in the Env trimer.

Most antibody-based therapies and strategies for eradication of HIV are based on neutralizing antibodies, and in particular broadly neutralizing antibodies. In contrast, strategies for a functional cure using non-neutralizing antibodies to target HIV-infected cells through Fc receptor effector function remain largely unexplored.

While current antiretroviral (ART) therapies are able to control viral replication, they are unable to fully restore health or a normal immune status. ART-treated individuals still experience several co-morbidities, including increased cardiovascular disease, bone disorders and cognitive impairment. Additionally, therapy interruption leads to the re-emergence of viral replication and AIDS progression. Therefore, a need exists to develop improved therapeutics for HIV infection and AIDS.

### SUMMARY

The present disclosure provides antibody conjugates that target Env and enable ADCC and/or ADCP killing of HIV-infected cells and methods of using the same to treat HIV infection, including antibody-based molecules that comprise an anti-CoRBS or anti-Cluster A antibody linked to a CD4 molecule to generate a molecule referred to as an Ab-CD4 or a CD4 mimetic molecule (CD4mc) to generate a molecule referred to as an Ab-CD4mc conjugate, as well as use of the Ab-CD4 or Ab-CD4mc conjugate for activating a direct neutralization and Fc-effector mediated effector activities of HIV virions and virally infected cells against epitope regions, traditionally known not to be involved in HIV neutralizing or antibody mediated elimination of HIV-infected cells.

In one aspect is disclosed an Ab-CD4 or Ab-CD4mc conjugate comprising an antibody, at least one linker, and at least one CD4 or CD4 mimetic compound, wherein the antibody binds to a Cluster A region or a co-receptor binding site (CoRBS) of the HIV envelope glycoprotein and comprises an Fc region, wherein the at least one linker links the antibody to the at least one CD4 or CD4 mimetic compound, and wherein the Ab-CD4 or Ab-CD4mc conjugate is capable of neutralizing an HIV virus and mediate Fc-effector activities of virions and HIV-1 infected cells.

In certain aspects disclosed herein, the antibody is a full-length antibody, and in certain aspects disclosed herein, the Ab-CD4 or Ab-CD4mc conjugate comprises an antibody, at least two linkers, and at least two CD4 or CD4 mimetic compounds, wherein a first linker links the antibody to a first CD4 or CD4mc compound and a second linker links the antibody to a second CD4 or CD4mc compound.

In certain aspects disclosed herein, the linker is (G₄Xaa)ₙ, wherein Xaa is serine or threonine and n is 2-16 (SEQ ID NO: 71), such as, for example, at least one linker selected from the group consisting of (G₄S)₆-(G₄T)₂ (SEQ ID NO: 72) and (G₄S)₈ (SEQ ID NO: 73). In certain embodiments, the at least one linker is 40-50 amino acids in length, and in certain embodiments, has a length ranging from about 50 Å to about 200 Å. In other embodiments disclosed herein, the at least one linker is a polyethylene glycol (PEG) linker, such as (PEG)ₙ wherein n is 4-100 or a subrange therein, such as 4-50.

In certain embodiments, the Cluster A antibody is selected from the group consisting of 2.2c, A32, C11, CH20, CH29, CH38, CH40, CH49, CH51, CH52, CH53, CH54, CH55, CH57, CH77, CH78, CH80, CH81, CH89, CH90, CH91, CH92, CH94, DH677.3, JR4, N12-i3, N5-i5, and N60-i3, such as A32 and N5-i5, and in certain embodiments, the CoRBS antibody is selected from the group consisting of 17b, 412d, 48d, E51, N12-i2, and X5, such as 17b and X5.

In certain embodiments, the antibody is conjugated to the at least one linker in the Fab region of the antibody, such as the C_{L}, C_{H}1, V_{L} or V_{H} regions, and in certain embodiments, the antibody is conjugated to the at least one linker in the Fc region of the antibody, such as the CH2 region or the CH3 region.

In certain embodiments, the at least one CD4 compound is selected from the group consisting of soluble CD4 (sCD4) and CD4 mimetic compounds (CD4mc). When the at least one CD4 compound is sCD4, disclosed herein are embodiments wherein the sCD4 compound consists of domains 1-4 of sCD4, domain 1 and domain 2 of sCD4, and domain 1 of sCD4. In various aspects of the disclosure, the Ab-CD4 neutralizes HIV virions.

In certain embodiments, disclosed herein is a vector comprising a nucleic acid molecule encoding any of the Ab-CD4 conjugates disclosed herein, and in certain embodiments, there is disclosed an isolated host cell comprising the vector.

Further disclosed herein are methods of killing HIV-infected cells through an Fc-mediated effector function, the method comprising contacting the HIV-infected cells with any Ab-CD4 or Ab-CD4mc conjugate described herein in the presence of immune cells that bind to the Fc region of the antibody and mediate the Fc-mediated effector function. In certain aspects, the Fc-mediated effector function is ADCC.

A further aspect is directed to a method of treating or preventing HIV infection in a subject, the method comprising administering to the subject an effective amount of a pharmaceutical composition comprising any Ab-CD4 or Ab-CD4mc conjugate disclosed herein. In certain embodiments, the antibody binds to the Cluster A region, and the method further comprises administering a second Ab-CD4 or Ab-CD4mc conjugate comprising an antibody that binds to the CoRBS. In certain embodiments, a mix of Ab-CD4 conjugates is used. In some aspects, the mix of Ab-CD4 or Ab-CD4mc conjugates comprise an Ab-CD4 or Ab-CD4mc conjugate comprising an antibody that binds to Cluster A region and Ab-CD4 or Ab-CD4mc conjugate comprising an antibody that binds to CoRBS. In certain embodiments, the pharmaceutical composition comprises a mix of Ab-CD4 or Ab-CD4mc conjugates comprising an antibody that binds to the CoRBS and an unconjugated antibody that binds to the Cluster A region. In certain embodiments, the pharmaceutical composition comprises a mix of Ab-CD4 or Ab-CD4mc conjugates comprising an antibody that binds to the CoRBS and an unconjugated antibody that binds to the CoRBS region. In certain embodiments, the pharmaceutical composition comprises a mix of Ab-CD4 or Ab-CD4mc conjugates comprising an antibody that binds to the Cluster A region and an unconjugated antibody that binds to the Cluster A region or alternatively, the pharmaceutical composition comprises a mix of Ab-CD4 or Ab-CD4mc conjugates comprising an antibody that binds to the Cluster A region and an unconjugated antibody that binds to the CoRBS region. In certain aspects of the method, the at least one CD4 compound is an sCD4 compound, which optionally consists of domains 1-4 of sCD4, domain 1 and domain 2 of sCD4, or domain 1 of sCD4. In certain aspects of the method, the at least one CD4 compound is a CD4 mimetic compound.

The method of treating or preventing HIV infection may, in certain embodiments, further comprise administering at least one anti-retroviral therapy, such as, for example, nucleoside analog reverse-transcriptase inhibitors, nucleotide reverse transcriptase inhibitors, non-nucleoside reverse transcriptase inhibitors, protease inhibitors, entry or fusion inhibitors, maturation inhibitors, and natural antivirals. In another aspect, there is provided a pharmaceutical composition comprising any Ab-CD4 conjugate as disclosed herein and a pharmaceutically acceptable carrier. In certain aspects of the disclosure, the Ab-CD4 or Ab-CD4mc conjugate of the pharmaceutical composition comprises a mix of Ab-CD4 or Ab-CD4mc conjugates as disclosed herein comprising Ab-CD4 or Ab-CD4mc comprising an antibody that binds Cluster A region and Ab-CD4 or Ab-CD4mc conjugate comprising an antibody that binds to CoRBS. In certain embodiments, the pharmaceutical composition comprises a mix of Ab-CD4 or Ab-CD4mc conjugate comprising an antibody that binds to the CoRBS and an unconjugated antibody that binds to the Cluster A region In certain embodiments, the pharmaceutical composition comprises a mix of Ab-CD4 or Ab-CD4mc conjugate comprising an antibody that binds to the CoRBS and an unconjugated antibody that binds to the CoRBS region In yet further embodiments, the pharmaceutical composition comprises a mix of Ab-CD4 or Ab-CD4mc conjugate comprising an antibody that binds to the Cluster A region and an unconjugated antibody that binds to the Cluster A region or alternatively, the pharmaceutical composition comprises a mix of Ab-CD4 or Ab-CD4mc conjugate comprising an antibody that binds to the Cluster A region and an unconjugated antibody that binds to the CoRBS region.

### BRIEF DESCRIPTION OF THE DRAWINGS

The accompanying drawings, which are included to provide a further understanding of the disclosure, are incorporated in and constitute a part of this specification. The drawings illustrate embodiments of the disclosure and, together with the detailed description, serve to explain the principles of the disclosure. No attempt is made to show structural details of the disclosure in more detail than may be necessary for a fundamental understanding of the disclosure and various ways in which it may be practiced.

FIG. 1A is a schematic illustrating an exemplary Ab-CD4 conjugate molecule wherein two sCD4 or CD4mc compounds are linked to a full-length Cluster A or CoRBS antibody via flexible linkers (GGGGS/T)ₙ (SEQ ID NO: 71) or (PEG)ₙ.

FIG. 1B are two schematics illustrating exemplary triggered and exposed CoRBS (top) or Cluster A (bottom) epitopes, wherein two gp120 trimers have been opened to allowed for six total binding sites per Env, wherein arrows indicate potential binding sites of CoRBS or Cluster A (three per Env trimer) and potential CD4 binding sites (three per Env trimer).

FIG. 2A is a bar graph showing recognition of cellular-expressed trimeric Env by 17b antibodies alone (left), a mixture of 17b antibodies and sCD4 (middle), and 17b-CD4 conjugate molecules (right), reported as normalized relative luminescent units (RLU), as described in Example 2.

FIG. 2B is a bar graph showing recognition of cellular-expressed trimeric Env by X5 antibodies alone (left), a mixture of X5 antibodies and sCD4 (middle), and X5-CD4 conjugate molecules (right), reported as normalized RLU, as described in Example 2.

FIG. 2C is a bar graph showing recognition of cellular-expressed trimeric Env by N5-i5 antibodies alone (left), a mixture of N5-i5 antibodies and sCD4 (middle), and N5-i5-CD4 conjugate molecules (right), reported as normalized RLU, as described in Example 2.

FIG. 2D is a bar graph showing recognition of cellular-expressed trimeric Env by A32 antibodies alone (left), a mixture of A32 antibodies and sCD4 (middle), and A32-CD4 conjugate molecules (right), reported as normalized RLU, as described in Example 2.

FIG. 3A is a bar graph showing the compiled median fluorescence intensities (MFI) of an infected (p24+) cell population with 17b antibodies alone (left), a mixture of 17b antibodies and sCD4 (middle), and 17b-CD4 conjugate molecules (right), as described in Example 2.

FIG. 3B is a bar graph showing the compiled MFI of an infected (p24+) cell population with X5 antibodies alone (left), a mixture of X5 antibodies and sCD4 (middle), and X5-CD4 conjugate molecules (right), as described in Example 2.

FIG. 3C is a bar graph showing the compiled MFI of an infected (p24+) cell population with N5-i5 antibodies alone (left), a mixture of N5-i5 antibodies and sCD4 (middle), and N5-i5-CD4 conjugate molecules (right), as described in Example 2.

FIG. 3D is a bar graph showing the compiled MFI of an infected (p24+) cell population with A32 antibodies alone (left), a mixture of A32 antibodies and sCD4 (middle), and A32-CD4 conjugate molecules (right), as described in Example 2.

FIG. 4A is a bar graph showing the compiled MFI of an infected (p24+) and mock-cell population with 17b-CD4 conjugate molecules (open circles), as described in Example 2.

FIG. 4B is a bar graph showing the compiled MFI of an infected (p24+) and mock-cell population with X5-CD4 conjugate molecules (open circles), as described in Example 2.

FIG. 4C is a bar graph showing the compiled MFI of an infected (p24+) and mock-cell population with N5-i5-CD4 conjugate molecules (open circles), as described in Example 2.

FIG. 4D is a bar graph showing the compiled MFI of an infected (p24+) and mock-cell population with A32-CD4 conjugate molecules (open circles), as described in Example 2.

FIG. 5 is a bar graph depicting the compiled MFI of primary CD4+ T cells infected with HIV-1 JRFL by Alexa-Fluor 647-conjugated F240 monoclonal antibodies in the presence of Ab-CD4 conjugates (17b-CD4, X5-CD4, N5-i5-CD4, and A32-CD4); antibody alone (10 μg/ml); or a mixture of antibody with sCD4 (10 μg/ml), as evaluated by flow cytometry and as described in Example 2.

FIG. 6A is a scatterplot depicting the percentage of ADCC obtained in the presence of the 17b antibody alone (left); a mixture of the 17b antibody and sCD4 (middle); or the 17b-CD4 conjugate (right), wherein primary CD4+ T cells infected with HIV-1 JRFL were used as targets and autologous PBMCs were used as effector cells in a FACS-based ADCC assay, as described in Example 3.

FIG. 6B is a scatterplot depicting the percentage of ADCC obtained in the presence of the N5-i5 antibody alone (left); a mixture of the N5-i5 antibody and sCD4 (middle); or the N5-i5-CD4 conjugate (right), wherein primary CD4+ T cells infected with HIV-1 JRFL were used as targets and autologous PBMCs were used as effector cells in a FACS-based ADCC assay, as described in Example 3.

FIG. 7A is a bar graph showing capacity of 17b antibody alone (left), a mixture of 17b antibody and sCD4 (middle), and 17b-CD4 conjugate (right) to capture VSV-G pseudotyped viral particle expressing HIV-1 mL Env, assessed by virus-capture assay as described in Example 4.

FIG. 7B is a bar graph showing capacity of X5 antibody alone (left), a mixture of X5 antibody and sCD4 (middle), and X5-CD4 conjugate (right) to capture VSV-G pseudotyped viral particle expressing HIV-1_{JRFL} Env, as assessed by virus-capture assay as described in Example 4.

FIG. 7C is a bar graph showing capacity of N5-i5 antibody alone (left), a mixture of N5-i5 antibody and sCD4 (middle), and N5-i5-CD4 conjugate (right) to capture VSV-G pseudotyped viral particle expressing HIV-1_{JRFL} Env, as assessed by virus-capture assay as described in Example 4.

FIG. 7D is a bar graph showing capacity of A32 antibody alone (left), a mixture of A32 antibody and sCD4 (middle), and A32-CD4 conjugate (right) to capture VSV-G pseudotyped viral particle expressing HIV-1_{JRFL} Env, as assessed by virus-capture assay as described in Example 4.

FIG. 8A is a graph showing the capacity to capture pseudotyped viral particle expressing HIV-1_{JRFL} Env after incubation with serial dilutions of 17b antibody alone (triangles), a mixture of 17b antibody and sCD4 (squares), and 17b-CD4 conjugate (circles), as described in Example 4.

FIG. 8B is a graph showing the capacity to capture pseudotyped viral particle expressing HIV-1_{JRFL} Env after incubation with serial dilutions of X5 antibody alone (triangles), a mixture of X5 antibody and sCD4 (squares), and X5-CD4 conjugate (circles), as described in Example 4.

FIG. 8C is a graph showing the capacity to capture pseudotyped viral particle expressing HIV-1_{JRFL} Env after incubation with serial dilutions of N5-i5 antibody alone (triangles), a mixture of N5-i5 antibody and sCD4 (squares), and N5-i5-CD4 conjugate (circles), as described in Example 4.

FIG. 8D is a graph showing the capacity to capture pseudotyped viral particle expressing HIV-1_{JRFL} Env after incubation with serial dilutions of A32 antibody alone (triangles), a mixture of A32 antibody and sCD4 (squares), and A32-CD4 conjugate (circles), as described in Example 4.

FIG. 9A is a graph showing the percent infectivity of A-MLV Env incubated with serial dilutions of 17b-CD4 conjugate (circles), X5-CD4 conjugate (diamonds), N5-i5 conjugate (triangles), and A32-CD4 conjugate (squares), as described in Example 4.

FIG. 9B is a graph showing the capacity to capture pseudotyped viral particle expressing HIV-1_{JRFL} Env after incubation with serial dilutions of sCD4 alone, as described in Example 4.

FIG. 10A is a bar graph showing recognition of cellular-expressed trimeric Env on wild-type cells or D368R cells (cells containing a D368R mutation in the CD4 binding site of HIV-1_{JRFL} Env) in the presence of 17b antibody alone (left), a mixture of 17b antibody and sCD4 (middle), and 17b-CD4 conjugate (right), as assessed by cell-based ELISA and described in Example 5.

FIG. 10B is a bar graph showing recognition of cellular-expressed trimeric Env on wild-type cells or D368R cells in the presence of X5 antibody alone (left), a mixture of X5 antibody and sCD4 (middle), and X5-CD4 conjugate (right), as assessed by cell-based ELISA and described in Example 5.

FIG. 10C is a bar graph showing recognition of cellular-expressed trimeric Env on wild-type cells or D368R cells in the presence of N5-i5 antibody alone (left), a mixture of N5-i5 antibody and sCD4 (middle), and N5-i5-CD4 conjugate (right), as assessed by cell-based ELISA and described in Example 5.

FIG. 10D is a bar graph showing recognition of cellular-expressed trimeric Env on wild-type cells or D368R cells in the presence of A32 antibody alone (left), a mixture of A32 antibody and sCD4 (middle), and A32-CD4 conjugate (right), as assessed by cell-based ELISA and described in Example 5.

FIG. 11A is a graph showing infectivity of viral particles pseudotyped with HIV-1_{JRFL} Env after incubations with serial dilutions of 17b-CD4 conjugates pre-incubated with 10 μg/mL of the gp120 D368R protein (diamonds); pre-incubated with 10 μg/mL of the ID2 gp120 protein (triangles); or in the absence of incubation (circles), as described in Example 5.

FIG. 11B is a graph showing infectivity of viral particles pseudotyped with HIV-1_{JRFL} Env after incubations with serial dilutions of X5-CD4 conjugates pre-incubated with 10 μg/mL of the gp120 D368R protein (diamonds); pre-incubated with 10 μg/mL of the ID2 gp120 protein (triangles); or in the absence of incubation (circles), as described in Example 5.

FIG. 11C is a graph showing infectivity of viral particles pseudotyped with HIV-1_{JRFL} Env after incubations with serial dilutions of N5-i5-CD4 conjugates pre-incubated with 10 μg/mL of the gp120 D368R protein (diamonds); pre-incubated with 10 μg/mL of the ID2 gp120 protein (triangles); or in the absence of incubation (circles), as described in Example 5.

FIG. 11D is a graph showing infectivity of viral particles pseudotyped with HIV-1_{JRFL} Env after incubations with serial dilutions of A32-CD4 conjugates pre-incubated with 10 μg/mL of the gp120 D368R protein (diamonds); pre-incubated with 10 μg/mL of the ID2 gp120 protein (triangles); or in the absence of incubation (circles), as described in Example 5.

FIG. 12A is a bar graph depicting the compiled MFI of primary CD4+ T cells infected with HIV-1 JRFL by 17b-CD4 conjugates pre-incubated with gp120 D368R protein (middle bar); pre-incubated with ID2 gp120 protein (right bar); or in the absence of incubation (left bar), as described in Example 5.

FIG. 12B is a bar graph depicting the compiled MFI of primary CD4+ T cells infected with HIV-1 JRFL by X5-CD4 conjugates pre-incubated with gp120 D368R protein (middle bar); pre-incubated with ID2 gp120 protein (right bar); or in the absence of incubation (left bar), as described in Example 5.

FIG. 12C is a bar graph depicting the compiled MFI of primary CD4+ T cells infected with HIV-1 JRFL by N5-i5-CD4 conjugates pre-incubated with gp120 D368R protein (middle bar); pre-incubated with ID2 gp120 protein (right bar); or in the absence of incubation (left bar), as described in Example 5.

FIG. 12D is a bar graph depicting the compiled MFI of primary CD4+ T cells infected with HIV-1 JRFL by A32-CD4 conjugates pre-incubated with gp120 D368R protein (middle bar); pre-incubated with ID2 gp120 protein (right bar); or in the absence of incubation (left bar), as described in Example 5.

FIG. 13A is a graph showing the plasma viral loads over time for HIV-1_{JRCSF}-infected hu-mice treated with a vehicle control, an A32-CD4 conjugate, an A32-CD4 conjugate comprising a GASDALIE mutation, or an A32-CD4 conjugate comprising a LALA mutation, as described in Example 6.

FIG. 13B is a plot showing the quantity of proviral DNA in CD4+ T cells immunopurified from blood and hematopoietic tissues (i.e., spleen and gut mucosa) at 10 days post-infection for HIV-1_{JRCSF}-infected hu-mice treated with a vehicle control, an A32-CD4 conjugate, an A32-CD4 conjugate comprising a GASDALIE mutation, or an A32-CD4 conjugate comprising a LALA mutation, as described in Example 6.

FIG. 13C is a graph showing the biodistribution of Ab-CD4 (μg/mL) in blood over time for HIV-1_{JRCSF}-infected hu-mice treated with a vehicle control, an A32-CD4 conjugate, an A32-CD4 conjugate comprising a GASDALIE mutation, or an A32-CD4 conjugate comprising a LALA mutation, as described in Example 6.

For all graphs, statistical significance was tested using an unpaired t-test or a Mann-Whitney U test based on statistical normality and is indicated as follows: *, P<0.05; **, P<0.01; ***, P<0.001; and ****, P<0.0001.

The drawings are not necessarily to scale, and may, in part, include exaggerated dimensions for clarity.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

The present disclosure provides antibody-based conjugate molecules comprising an anti-CoRBS or anti-Cluster A antibody IgG linked to CD4 or CD4 mimic (CD4mc) compounds to generate single-chain molecules referred to herein as Ab-CD4 conjugates. As demonstrated herein, the Ab-CD4 conjugates can efficiently target and eliminate HIV-infected cells by ADCC through a mechanism involving the binding of both the antibodies and the CD4 moieties to binding sites on the HIV Env glycoprotein. Furthermore, both the CoRBS and the Cluster A Ab-CD4 conjugates are capable of impacting HIV through a coordinated mechanism of direct neutralization of virions and infected cells clearance by Fc-effector functions, including ADCC and ADCP. Accordingly, disclosed herein is a method for activating a direct neutralization of HIV virions using antibodies that may or may not have neutralizing activities. The Ab-CD4 conjugates can be made by using the non-neutralizing and neutralizing antibodies that target CoRBS or Cluster A and induce Fc-effector function activities.

### Definitions

In order that the present invention may be more readily understood, certain terms are first defined. Additional definitions are set forth throughout the detailed description.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps. When used herein, the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “HIV” refers to human immunodeficiency virus. HIV can be classified into two major subtypes (HIV-1 and HIV-2), each of which has many subtypes. In some embodiments, a human subject is infected with the HIV-1 or HIV-2 subtypes.

As used herein, “antibody-dependent cellular phagocytosis (ADCP)” refers to an immune response wherein an Fc receptor-dependent function of antibody-dependent cellular phagocytosis provides mechanisms for clearance of virus and virus-infected cells by cells including monocytes and macrophages, as well as for stimulation of downstream adaptive immune responses by facilitating antigen presentation, or by stimulating the secretion of inflammatory mediators.

As used herein, “antibody-dependent cellular cytotoxicity (ADCC)” refers to an immune response mediated by an effector cell (e.g., a natural killer (NK) cell) of the immune system that lyses a target cell having membrane-bound surface antigens, wherein the target cell antigens have been bound by specific antibodies.

The term “antibody” refers to an immunoglobulin or antigen-binding fragment thereof, and encompasses any polypeptide comprising an antigen-binding fragment or an antigen-binding domain. The term includes but is not limited to polyclonal, monoclonal, monospecific, polyspecific, humanized, human, single-chain, chimeric, synthetic, recombinant, hybrid, mutated, grafted, and in vitro generated antibodies. Unless preceded by the word “intact,” the term “antibody” includes antibody fragments such as Fab, F(ab′)₂, Fv, scFv, Fd, dAb, and other antibody fragments that retain antigen-binding function. Unless otherwise specified, an antibody is not necessarily from any particular source, nor is it produced by any particular method.

In certain embodiments, an antibody disclosed herein is a non-neutralizing antibody. In certain embodiments, an antibody disclosed herein is a neutralizing antibody.

In certain embodiments, an antibody can be any of the five major classes of immunoglobulins, including IgA, IgD, IgE, IgG, and IgM, or subclasses thereof, based on the identity of their heavy-chain constant domains, which are referred to as alpha, delta, epsilon, gamma, and mu, respectively. In certain embodiments, the antibody is an IgG antibody.

The basic four-chain antibody unit is a heterotetrameric glycoprotein composed of two identical light chains and two identical heavy chains. In the case of an IgG antibody, the 4-chain unit is generally about 150,000 daltons. Each light chain is linked to a heavy chain by one covalent disulfide bond, while the two heavy chain are linked to each other by one or more disulfide bonds, depending on the heavy chain isotype. Each heavy and light chain also has regularly spaced intrachain disulfide bridges.

The term “non-neutralizing antibody” refers to an antibody that binds to a viral antigen but does not directly decrease or disrupt viral entry into a cell. A non-neutralizing antibody may, in certain embodiments, have variable activity in mediating ADCC and/or ADCP.

The term “neutralizing antibody” refers to an antibody that binds to a viral antigen and directly decreases or disrupts viral entry into a cell. A neutralizing antibody may inhibit the entry of HIV with a neutralization index of, for example, >1.5 or >2.0, discussed in Kostrikis, L. G. et al., J. Virol. 1996; 70(1):445-458.

The term “monoclonal antibody” refers to a homogeneous antibody population involved in the highly specific recognition and binding of a single antigenic determinant or epitope. The term “monoclonal antibody” encompasses both intact and full-length monoclonal antibodies, as well as antibody fragments (such as Fab, Fab′, F(ab′)2, and Fv), single chain (scFV) mutants, fusion proteins comprising an antibody portion, and any other modified immunoglobulin molecule comprising an antigen recognition site. Furthermore, “monoclonal antibody” refers to such antibodies made in any number of manners, including but not limited to by hybridoma, phage selection, recombinant expression, and transgenic animals.

As used herein, the term “intact antibody,” or a “full-length antibody,” refers to an antibody that comprises an antigen-binding variable region, as well as a light chain constant domain (C_{L}) and heavy chain constant domains, C_{H}1, C_{H}2, C_{H}3, and C_{H}4, as appropriate for the antibody class. An intact or full-length antibody includes the Fc (Fragment, crystallizable) region, which comprises two heavy chains that contribute two or three constant domains depending on the class of the antibody. In certain embodiments, an intact antibody may have one or more effector functions, which refers to those biological activities attributable to the Fc region of an antibody, including, for example, complement dependent cytotoxicity, ADCC, and ADCP.

The term “antigen” refers to a substance, such as a protein, a fragment thereof or a polysaccharide linked to a protein carrier, that when expressed in an animal or human cell or tissue is capable of triggering an immune response. The protein or fragment thereof may be glycosylated or non-glycosylated.

The term “conjugate” refers to at least two molecules that are covalently linked to each other via at least one linker, which linker can include any known linker in the art, such as peptides, polyethylene glycol, and chemically-modified amino acids, wherein the at least two molecules may be covalently joined together after individual expression or may be expressed as a single molecule via a vector. In certain embodiments, a conjugate comprises at least three molecules covalently linked to each other via at least two linkers, such as at least two CD4 compounds each independently linked to a Cluster A or co-receptor binding site antibody via a linker.

The term “mimetic” refers to a compound, such as a CD4 small molecule mimetic or CD4 peptide mimetic, that can bind to certain receptor binding sites within the HIV envelope glycoprotein but is not structurally related to the original compound it mimics, such as a CD4 molecule.

The term “CD4 compound” refers to a soluble CD4 peptide or a CD4 small molecule mimetic compound or a CD4 peptide mimetic.

The term “epitope” refers to a portion of an antigen capable of being recognized and specifically bound by a particular antibody. When the antigen is a polypeptide, epitopes can be formed both from contiguous amino acids and noncontiguous amino acids juxtaposed by tertiary folding of a protein.

The term “binding affinity” refers to the strength of the sum total of noncovalent interactions between a single binding site of a molecule (e.g., an antibody) and its binding partner (e.g., an antigen). As used herein, “binding affinity” indicates the intrinsic binding affinity which reflects 1:1 interaction between members of a binding pair (e.g., antibody and antigen). The affinity of a molecule for its partner may be represented by the dissociation constant (Kd). Low-affinity antibodies generally bind antigen slowly and tend to dissociate readily, while high-affinity antibodies generally bind antigen faster and tend to remain bound longer. The affinity of an antibody for an antigen can be determined experimentally using any suitable method known in the art, including, for example, flow cytometry or enzyme-linked immunosorbent assay (ELISA).

The term “subject” refers to any animal, such as a mammal, including humans, non-human primates, rodents, and the like which is to be the recipient of a particular treatment.

The term “pharmaceutically acceptable carrier” or “pharmaceutically acceptable excipient” means solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. In certain embodiments, the pharmaceutically acceptable carrier or excipient is not naturally occurring.

The term “preventing” when used in the context of a disease or disease condition means prophylactic administration of a composition that stops or otherwise delays the onset of a pathological hallmark or symptom of a disease or disorder.

The term “treating” when used in the context of a disease or disease condition means ameliorating, improving or remedying a disease, disorder, or symptom of a disease or condition associated with the disease, or can mean completely or partially stopping, on a molecular level, the biochemical basis of the disease, such as halting replication of a virus, etc.

The term “therapeutically effective amount” when used in the context of an amount of an active agent means an amount that results in an improvement or remediation of the disease, disorder, or symptoms of the disease or condition.

Fc receptor-dependent effector functions such as ADCC and ADCP constitute a bridge between innate and adaptive immunities to HIV and may trigger a clearing of virus particles or virus-infected cells through mechanisms involving interactions of antibody constant (Fc) regions and Fcγ receptors (FcγRs) on the surface of cells involved or potentially involved in HIV infection, such as natural killer cells, monocytes, macrophages, dendritic cells, and neutrophils. In contrast to broadly neutralizing antibody responses, which become detectable 2-4 years post-infection, ADCC and ADCP responses appear relatively early during acute infection, and may be detectable as early as 48 days after acute HIV infection. These early ADCC and ADCP responses, which in general are broadly reactive, precede the appearance of neutralizing antibody responses with similar breadth, and ADCC and ADCP ultimately lead to the target cell being killed by an effector cell, decreasing the probability of cell-to-cell transmission of the virus.

Considerable evidence supports a role of ADCC in preventing or modulating HIV infection. ADCC responses in chronically HIV infected individuals have been shown to correlate with a slower progression of HIV and decreased virus replication. The ability to induce ADCC is therefore important in protecting against HIV transmission and in treating HIV-infected individuals.

HIV envelop protein (Env), the only viral protein present on the surface of HIV virions and HIV infect cells, is initially synthesized as a longer precursor protein, gp160. Gp160 forms a homotrimer, and in vivo, the gp160 glycoprotein is processed to the mature envelop glycoproteins gp120 and gp41, which are noncovalently associated with each other in a complex on the surface of the virus. The gp120 surface protein contains a high affinity binding site for human CD4, the primary receptor for HIV, as well as domains that interact with fusion coreceptors CCR5 and CXCR4. The gp41 protein spans the viral membrane and contains a sequence of amino acids for the fusion of viral and cellular membranes. The Env complex is a trimeric structure composed of three gp120 and three gp41 subunits. The CD4 binding site and the co-receptor binding site (CoRBS) are located in the gp41 components. The gp120 subunit contains epitopes known as the A32-region or Cluster A epitopes, including the constant region 1 and 2 (C1C2). These regions in unliganded Env trimers (closed conformation) are important for trimer stability and are inaccessible for antibody recognition until interactions of the Env spike with the cellular CD4 receptor. Exposure of these targets in infected cells is limited by the low levels of surface CD4 that could effectively trigger Env trimers emerging on the cell surface, representing a highly sophisticated mechanism put in place by HIV to prevent antibody-mediated clearance of virally-infected cells. Cluster A antibodies are antibodies that recognize C1C2 of the gp120 subunit [20].

Currently, among the most prominent targets for non-neutralizing antibodies are epitopes that become available only after CD4 binding (CD4-induced or CD4i epitopes), when the Env protein transitions from its unliganded “closed” conformation (State 1) to its “open” CD4-bound conformation (State 3) [14]. Both the CoRBS epitope and the Cluster A epitope, which are located within the highly conserved HIV Env regions, become available sequentially in this process after CD4 binding [12, 15-24]. Whereas the CoRBS is localized at the surface of the Env trimer, mapping to the outer domain of gp120, proximal to the CD4 binding site, the Cluster A epitopes map to the interior of Env within the C1C2 regions of gp120 at the gp41-gp120 interface. Cluster A epitopes are directly involved in inter-promoter contacts that stabilize the trimer. The exposure of the Cluster A region requires a structural rearrangement of the gp120 and gp41 subunits, which occurs late in the entry process as a consequence of CD4-induced changes in Env [12]. Therefore, most known Cluster A antibodies uniformly lack neutralizing activities [20]. CD4i non-neutralizing antibodies are frequently elicited in HIV-infected individuals and are capable of mediating potent ADCC against CD4i targets [16, 18, 25, 26]. Unfortunately, their potential as ADCC mediators is diminished by the fact that the cells infected with primary HIV isolates express Env in its “closed” conformation, in which CD4i targets are not accessible for antibody recognition [14, 27, 28].

Soluble CD4 (sCD4) and CD4-mimetic (CD4mc), the later including small molecule compounds and CD4 peptide mimics have been used in an attempt to modulate Env conformation and expose CD4i epitopes, for example within the CoRBS for direct neutralizing activity [36]. sCD4 and CD4mc have also been used to expose CD4i Env epitopes on infected cells, effectively sensitizing them to ADCC by CD4i non-neutralizing antibodies present in sera, breastmilk, and mucosa of HIV-infected individuals [41]. However, the mechanism of sensitization of infected cells to ADCC uses a sequential opening of the Env trimer and depends on the cooperation of sCD4/CD4mc in addition to CoRBS and Cluster A non-neutralizing antibodies [22, 24, 30]. Thus, a mixture of sCD4 or CD4mc, a CoRBS antibody, and a Cluster A antibody is used to trigger the asymmetric ADCC vulnerable Env conformation referred to as “State 2A,” an intermediate between State 1 and State 3 in the opening of the Env trimer [22, 24, 42].

As disclosed herein, however, such mixtures are less effective at neutralizing HIV and sensitizing HIV-infected cells to ADCC. The Examples provided herein demonstrate the superiority of the Ab-CD4 conjugates disclosed herein over mixtures containing an unconjugated non-neutralizing or neutralizing antibody and a CD4 compound. While not wishing to be bound by theory, it is believed that non-conjugated mixtures of a CD4 compound and a non-neutralizing antibody are unable to maintain the Cluster A-region epitopes exposed (e.g., State 2A and 3 Env conformations) for sufficient time to allow for effective antibody binding.

Disclosed herein are non-neutralizing antibody-based conjugate molecules that physically combine these elements in such a way to overcome the unfavorable steric exposure of the Cluster A-region epitopes and efficiently eliminate HIV-infected cells by ADCC and/or ADCP. Also disclosed herein are neutralizing antibody-based conjugate molecules that physically combine these elements in such a way to overcome the unfavorable steric exposure of the Cluster A-region epitopes and efficiently eliminate HIV-infected cells by ADCC and/or ADCP. The Ab-CD4 conjugates provided herein comprise at least one CD4 compound linked to a Cluster A or CoRBS antibody via a flexible linker. These Ab-CD4 conjugate molecules can be used in methods of treating HIV infection through the use of non-neutralizing or neutralizing antibodies to eliminate HIV virions and HIV-infected cells through neutralization and Fc receptor effector function. This effect results from the cooperative action of the two moieties of the conjugate, wherein the CD4 moiety binds within the CD4 binding site of the Env trimer and triggers it to assume the CD4-bound conformation required to expose the Cluster A region epitope, to which the non-neutralizing antibody can then bind. The non-neutralizing antibody moiety then binds to the exposed epitope, effectively mediating neutralization and the Fc receptor effector function.

In certain embodiments, the Ab-CD4 conjugate molecules disclosed herein can be used in methods of treating HIV infection through the use of neutralizing antibodies that eliminate HIV-infected cells through neutralization and Fc receptor effector function.

### Cluster A or CoRBS Antibodies

In embodiments of the disclosure, any non-neutralizing or neutralizing antibody may be used in the Ab-CD4 conjugates disclosed herein, including, for example, at least one non-neutralizing antibody selected from the group consisting of 2.2c, A32, C11, CH20, CH29, CH38, CH40, CH49, CH51, CH52, CH53, CH54, CH55, CH57, CH77, CH78, CH80, CH81, CH89, CH90, CH91, CH92, CH94, DH677.3, JR4, N12-i3, N5-i5, N60-i3, 17b, 412d, 48d, E51, N12-i2, and X5. In certain embodiments, the Ab-CD4 conjugate comprises at least one neutralizing antibody. In certain embodiments, the neutralizing or non-neutralizing antibody is an IgG antibody, and in certain embodiments, the IgG antibody is a full-length or intact antibody. In various embodiments of the disclosure, the Ab-CD4 conjugate comprises at least one non-neutralizing antibody selected from Cluster A antibodies and CoRBS antibodies.

Cluster A antibodies are known to recognize highly conserved epitope surfaces within the inner domain of C1C2 region of gp120 [20]. Several Cluster A antibodies are known in the art and include, for example, 2.2c (having a heavy chain sequence of SEQ ID NO: 1 and a light chain sequence of SEQ ID NO: 2 or having the 6 CDRs of SEQ ID NOs: 1 and 2), A32 (having a heavy chain sequence of SEQ ID NO: 3 and a light chain sequence of SEQ ID NO: 4 or having the 6 CDRs of SEQ ID NOs: 3 and 4), C11 (having a heavy chain sequence of SEQ ID NO: 5 and a light chain sequence of SEQ ID NO: 6 or having the 6 CDRs of SEQ ID NOs: 5 and 6), CH20 (having a heavy chain sequence of SEQ ID NO: 7 and a light chain sequence of SEQ ID NO: 8 or having the 6 CDRs of SEQ ID NOs: 7 and 8), CH29 (having a heavy chain sequence of SEQ ID NO: 9 and a light chain sequence of SEQ ID NO: 10 or having the 6 CDRs of SEQ ID NOs: 9 and 10), CH38 (having a heavy chain sequence of SEQ ID NO: 11 and a light chain sequence of SEQ ID NO: 12 or having the 6 CDRs of SEQ ID NOs: 11 and 12), CH40 (having a heavy chain sequence of SEQ ID NO: 13 and a light chain sequence of SEQ ID NO: 14 or having the 6 CDRs of SEQ ID NOs: 13 and 14), CH49 (having a heavy chain sequence of SEQ ID NO: 15 and a light chain sequence of SEQ ID NO: 16 or having the 6 CDRs of SEQ ID NOs: 15 and 16), CH51 (having a heavy chain sequence of SEQ ID NO: 17 and a light chain sequence of SEQ ID NO: 18 or having the 6 CDRs of SEQ ID NOs: 17 and 18), CH52 (having a heavy chain sequence of SEQ ID NO: 19 and a light chain sequence of SEQ ID NO: 20 or having the 6 CDRs of SEQ ID NOs: 19 and 20), CH53 (having a heavy chain sequence of SEQ ID NO: 21 and a light chain sequence of SEQ ID NO: 22 or having the 6 CDRs of SEQ ID NOs: 21 and 22), CH54 (having a heavy chain sequence of SEQ ID NO: 23 and a light chain sequence of SEQ ID NO: 24 or having the 6 CDRs of SEQ ID NOs: 23 and 24), CH55 (having a heavy chain sequence of SEQ ID NO: 25 and a light chain sequence of SEQ ID NO: 26 or having the 6 CDRs of SEQ ID NOs: 25 and 26), CH57 (having a heavy chain sequence of SEQ ID NO: 27 and a light chain sequence of SEQ ID NO: 28 or having the 6 CDRs of SEQ ID NOs: 27 and 28), CH77 (having a heavy chain sequence of SEQ ID NO: 29 and a light chain sequence of SEQ ID NO: 30 or having the 6 CDRs of SEQ ID NOs: 29 and 30), CH78 (having a heavy chain sequence of SEQ ID NO: 31 and a light chain sequence of SEQ ID NO: 32 or having the 6 CDRs of SEQ ID NOs: 31 and 32), CH80 (having a heavy chain sequence of SEQ ID NO: 33 and a light chain sequence of SEQ ID NO: 34 or having the 6 CDRs of SEQ ID NOs: 33 and 34), CH81 (having a heavy chain sequence of SEQ ID NO: 35 and a light chain sequence of SEQ ID NO: 36 or having the 6 CDRs of SEQ ID NOs: 35 and 36), CH89 (having a heavy chain sequence of SEQ ID NO: 37 and a light chain sequence of SEQ ID NO: 38 or having the 6 CDRs of SEQ ID NOs: 37 and 38), CH90 (having a heavy chain sequence of SEQ ID NO: 39 and a light chain sequence of SEQ ID NO: 40 or having the 6 CDRs of SEQ ID NOs: 39 and 40), CH91 (having a heavy chain sequence of SEQ ID NO: 41 and a light chain sequence of SEQ ID NO: 42 or having the 6 CDRs of SEQ ID NOs: 41 and 42), CH92 (having a heavy chain sequence of SEQ ID NO: 43 and a light chain sequence of SEQ ID NO: 44 or having the 6 CDRs of SEQ ID NOs: 43 and 44), CH94 (having a heavy chain sequence of SEQ ID NO: 45 and a light chain sequence of SEQ ID NO: 46 or having the 6 CDRs of SEQ ID NOs: 45 and 46), DH677.3 (having a heavy chain sequence of SEQ ID NO: 47 and a light chain sequence of SEQ ID NO: 48 or having the 6 CDRs of SEQ ID NOs: 47 and 48), JR4 (having a heavy chain sequence of SEQ ID NO: 49 and a light chain sequence of SEQ ID NO: 50 or having the 6 CDRs of SEQ ID NOs: 49 and 50), N12-i3 (having a heavy chain sequence of SEQ ID NO: 51 and a light chain sequence of SEQ ID NO: 52 or having the 6 CDRs of SEQ ID NOs: 51 and 52), N5-i5 (having a heavy chain sequence of SEQ ID NO: 53 and a light chain sequence of SEQ ID NO: 54 or having the 6 CDRs of SEQ ID NOs: 53 and 54), and N60-i3 (having a heavy chain sequence of SEQ ID NO: 55 and a light chain sequence of SEQ ID NO: 56 or having the 6 CDRs of SEQ ID NOs: 55 and 56), as well as those disclosed, for example, in U.S. Published Patent Application No. 2017/0283486, incorporated by reference herein in its entirety. The heavy and light chain amino acid sequences of exemplary Cluster A antibodies are shown below in Table A.

As disclosed in U.S. Published Patent Application No. 2017/0283486, these antibodies cross-compete with each other for binding to monomeric gp120. Monoclonal antibodies N5-i5, N60-i3, 2.2c, and JR4, for example, bind to largely overlapping areas of gp120, proximal to the N- and C-terminal extensions and within the C1 and C2 regions. These antibodies approach gp120 from slightly different angles and target their epitopes by two binding modes shown by a heavy and light chain variable region switch. These regions are known to interact with gp41 in untriggered trimeric Env and thus are inaccessible for antibody recognition until interactions of Env with the host receptor CD4.

TABLE 1
Cluster A Antibody Polypeptide Sequences

| | SEQ | | SEQ | |
|---|---|---|---|---|
| Antibody | ID | Heavy chain amino acid | ID | Light chain amino acid |
| name | NO. | sequence | NO. | sequence |
| 2.2c | 1 | QVQLQQWGAGLLKPSETLSLTCGVY | 2 | IQMTQSPSFVSASVGDRVTI |
| | | GESLSGHYWSWVRQPPGKRLEWIGE | | TCRASQGISSYLAWYQQKPG |
| | | IKHNGSPNYHPSLKSRVTISLDMSK | | KAPKLVIYAASTLQSGVPSR |
| | | NQFSLNLTSVTAADTAVYFCARRSN | | FSGSGSGTEFTLTISSLQPE |
| | | WPYLPFDPWGQGTLVTVSSASTKGP | | DFATYYCQHLIGLRSFGQGT |
| | | SVFPLAPSSKSTSGGTAALGCLVKD | | KVEIKRTVAAPSVFIFPPSD |
| | | YFPEPVTVSWNSGALTSGVHTFPAV | | EQLKSGTASVVCLLNNFYPR |
| | | LQSSGLYSLSSVVTVPSSSLGTQTY | | EAKVQWKVDNALQSGNSQES |
| | | ICNVNHKPSNTKVDKRVEPKSCDKT | | VTEQDSKDSTYSLSSTLTLS |
| | | HTCPPCPAPELLGGPSVFLFPPKPK | | KADYEKHKVYACEVTHQGLS |
| | | DTLMISRTPEVTCVVVDVSHEDPEV | | SPVTKSFNRGEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| A32 | 3 | QVQLQESGPGLVKPSQTLSLSCTVS | 4 | VLTQPPSASGSPGQSVTISC |
| | | GGSSSSGAHYWSWIRQYPGKGLEWI | | TGTSSDVGGYNYVSWYQHHP |
| | | GYIHYSGNTYYNPSLKSRITISQHT | | GKAPKLIISEVNNRPSGVPD |
| | | SENQFSLKLNSVTVADTAVYYCARG | | RFSGSKSGNTASLTVSGLQA |
| | | TRLRTLRNAFDIWGQGTMVTVSSAS | | EDEAEYYCSSYTDIHNFVFG |
| | | TKGPSVFPLAPSSKSTSGGTAALGC | | GGTKLTVLGQPKAAPSVTLF |
| | | LVKDYFPEPVTVSWNSGALTSGVHT | | PPSSEELQANKATLVCLISD |
| | | FPAVLQSSGLYSLSSVVTVPSSSLG | | FYPGAVTVAWKADSSPVKAG |
| | | TQTYICNVNHKPSNTKVDKRVEPKS | | VETTTPSKQSNNKYAASSYL |
| | | CDKTHTCPPCPAPELLGGPSVFLFP | | SLTPEQWKSHRSYSCQVTHE |
| | | PKPKDTLMISRTPEVTCVVVDVSHE | | GSTVEKTVAPTEC |
| | | DPEVKFNWYVDGVEVHNAKTKPREE | | |
| | | QYNSTYRVVSVLTVLHQDWLNGKEY | | |
| | | KCKVSNKALPAPIEKTISKAKGQPR | | |
| | | EPQVYTLPPSREEMTKNQVSLTCLV | | |
| | | KGFYPSDIAVEWESNGQPENNYKTT | | |
| | | PPVLDSDGSFFLYSKLTVDKSRWQQ | | |
| | | GNVFSCSVMHEALHNHYTQKSLSLS | | |
| | | PGK | | |
| C11 | 5 | EVQLVESGGGLVKPGGSLRLSCAAS | 6 | DIVMTQSPLSLPVTPGEPAS |
| | | GFTFSSYSMNWVRQAPGRGLEWVSS | | ISCRSSQSLLHSNGYNYLDW |
| | | ISNTSTYIYYADSVEGRFTLSRDNA | | YLQKPGQSPQLLIYLGSNRA |
| | | KNSLYLQMNSLRAEDTAVYYCARAN | | SGVPDRFIGSGSGTDFTLKI |
| | | QHFDWLLSLLGGYHYYGMDVWGQGT | | SRVEAEDVGVFYCMQALQAV |
| | | TVTVSSASTKGPSVFPLAPSSKSTS | | GFGPGTKVEIKRTVAAPSVF |
| | | GGTAALGCLVKDYFPEPVTVSWNSG | | IFPPSDEQLKSGTASVVCLL |
| | | ALTSGVHTFPAVLQSSGLYSLSSVV | | NNFYPREAKVQWKVDNALQS |
| | | TVPSSSLGTQTYICNVNHKPSNTKV | | GNSQESVTEQDSKDSTYSLS |
| | | DKRVEPKSCDKTHTCPPCPAPELLG | | STLTLSKADYEKHKVYACEV |
| | | GPSVFLFPPKPKDTLMISRTPEVTC | | THQGLSSPVTKSFNRGEC |
| | | VVVDVSHEDPEVKFNWYVDGVEVHN | | |
| | | AKTKPREEQYNSTYRVVSVLTVLHQ | | |
| | | DWLNGKEYKCKVSNKALPAPIEKTI | | |
| | | SKAKGQPREPQVYTLPPSREEMTKN | | |
| | | QVSLTCLVKGFYPSDIAVEWESNGQ | | |
| | | PENNYKTTPPVLDSDGSFFLYSKLT | | |
| | | VDKSRWQQGNVFSCSVMHEALHNHY | | |
| | | TQKSLSLSPGK | | |
| CH20 | 7 | QVQLVQSGAEVQKPGSSVKVSCKAS | 8 | QSALTQPASVSGSPGQSITI |
| | | GGTFSTYSISWVRQAPGQGLEWMGR | | SCTGTSSDVGSYNLVSWYQQ |
| | | SIPILGIANYAQKFQGRVTFTADKS | | HPGKAPKLMIYEVSKRPSGV |
| | | TTTAYMELSSLRSEDTAVYYCARGV | | SNRFSGSKSGNTASLTISGL |
| | | GQQLVQYYFDYWGQGTLVTVSSAST | | LAEDEADYYCCSYAGSSISV |
| | | KGPSVFPLAPSSKSTSGGTAALGCL | | FGGGTKLTVLGQPKAAPSVT |
| | | VKDYFPEPVTVSWNSGALTSGVHTF | | LFPPSSEELQANKATLVCLI |
| | | PAVLQSSGLYSLSSVVTVPSSSLGT | | SDFYPGAVTVAWKADSSPVK |
| | | QTYICNVNHKPSNTKVDKRVEPKSC | | AGVETTTPSKQSNNKYAASS |
| | | DKTHTCPPCPAPELLGGPSVFLFPP | | YLSLTPEQWKSHRSYSCQVT |
| | | KPKDTLMISRTPEVTCVVVDVSHED | | HEGSTVEKTVAPTEC |
| | | PEVKFNWYVDGVEVHNAKTKPREEQ | | |
| | | YNSTYRVVSVLTVLHQDWLNGKEYK | | |
| | | CKVSNKALPAPIEKTISKAKGQPRE | | |
| | | PQVYTLPPSREEMTKNQVSLTCLVK | | |
| | | GFYPSDIAVEWESNGQPENNYKTTP | | |
| | | PVLDSDGSFFLYSKLTVDKSRWQQG | | |
| | | NVFSCSVMHEALHNHYTQKSLSLSP | | |
| | | GK | | |
| CH29 | 9 | QVQLVQSGAEVKKPGASVKVSCKAS | 10 | IVLTQSPSSLSASVGDRVTI |
| | | GYTFTGYYIHWVRQAPGQGLEWMGW | | TCRASQSISSYLNWYQQKPG |
| | | INPNSGGTNYAQMFQGRVTMTRDTS | | KAPKLLIYAASSLQSGVPSR |
| | | ISTAYMELSRLRSDDTAVFYCATGG | | FSGSGSGTDFTLTISSLQPD |
| | | SWLGGVDYWGQGTLVTVSSASTKGP | | DFATYYCQRSYSTPLTFGQG |
| | | SVFPLAPSSKSTSGGTAALGCLVKD | | TKVEIKRTVAAPSVFIFPPS |
| | | YFPEPVTVSWNSGALTSGVHTFPAV | | DEQLKSGTASVVCLLNNFYP |
| | | LQSSGLYSLSSVVTVPSSSLGTQTY | | REAKVQWKVDNALQSGNSQE |
| | | ICNVNHKPSNTKVDKRVEPKSCDKT | | SVTEQDSKDSTYSLSSTLTL |
| | | HTCPPCPAPELLGGPSVFLFPPKPK | | SKADYEKHKVYACEVTHQGL |
| | | DTLMISRTPEVTCVVVDVSHEDPEV | | SSPVTKSFNRGEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| CH38 | 11 | EVQLVESGGGLVQPGGSLRLSCAAS | 12 | QSALTQPASVSGSPGQSITI |
| | | GFTFSNNAMSWVRQTPGKGLEWVSS | | SCTGTSSDVGTYNSVSWYQQ |
| | | FSGGRDTTYYADSVKGRFTISRDDS | | HPGKAPNLIIYDVTNRPSGV |
| | | KNTLFLQMSSLRAEDTAVYYCAKDL | | SNRFSGSKSGNTASLTISGL |
| | | GLLRGIANWGQGTLVTVSSASTKGP | | QTEDEADYYCSSYRRTNTLG |
| | | SVFPLAPSSKSTSGGTAALGCLVKD | | FGGGTKLTVLGQPKAAPSVT |
| | | YFPEPVTVSWNSGALTSGVHTFPAV | | LFPPSSEELQANKATLVCLI |
| | | LQSSGLYSLSSVVTVPSSSLGTQTY | | SDFYPGAVTVAWKADSSPVK |
| | | ICNVNHKPSNTKVDKRVEPKSCDKT | | AGVETTTPSKQSNNKYAASS |
| | | HTCPPCPAPELLGGPSVFLFPPKPK | | YLSLTPEQWKSHRSYSCQVT |
| | | DTLMISRTPEVTCVVVDVSHEDPEV | | HEGSTVEKTVAPTEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| CH40 | 13 | QVQLVQSGAEVKKPGASVKLSCKAS | 14 | EIVLTQSPGTLSLSPGERAT |
| | | GYTFNSYYINWLRQAPGQGLEWMGI | | LSCRASQSVSSRSLAWYQQK |
| | | INPSSSSTNYAQNFQGRVTMTRDTS | | PGQAPRLLIYGASSRATGIP |
| | | TSTVYMELSSLRSEDTAVYYCARNY | | DRFSGSGSGTDFTLTISRLE |
| | | AGIEARGWLDPWGQGTLVTVSSAST | | PEDFAVYYCQQSTTFGGGTK |
| | | KGPSVFPLAPSSKSTSGGTAALGCL | | VEIKRTVAAPSVFIFPPSDE |
| | | VKDYFPEPVTVSWNSGALTSGVHTF | | QLKSGTASVVCLLNNFYPRE |
| | | PAVLQSSGLYSLSSVVTVPSSSLGT | | AKVQWKVDNALQSGNSQESV |
| | | QTYICNVNHKPSNTKVDKRVEPKSC | | TEQDSKDSTYSLSSTLTLSK |
| | | DKTHTCPPCPAPELLGGPSVFLFPP | | ADYEKHKVYACEVTHQGLSS |
| | | KPKDTLMISRTPEVTCVVVDVSHED | | PVTKSFNRGEC |
| | | PEVKFNWYVDGVEVHNAKTKPREEQ | | |
| | | YNSTYRVVSVLTVLHQDWLNGKEYK | | |
| | | CKVSNKALPAPIEKTISKAKGQPRE | | |
| | | PQVYTLPPSREEMTKNQVSLTCLVK | | |
| | | GFYPSDIAVEWESNGQPENNYKTTP | | |
| | | PVLDSDGSFFLYSKLTVDKSRWQQG | | |
| | | NVFSCSVMHEALHNHYTQKSLSLSP | | |
| | | GK | | |
| CH40 | 15 | QVQLVQSGAEVKKPGASVKVSCKAS | 16 | QSALTQPRSVSGSPGQSVTI |
| | | EYSFTGYYLHWVRQAPGQGLEWMGW | | SCTGTSSDVGSYTYVSWYQQ |
| | | INPNNGDTRSAQRFQGRVTMTRDTS | | HPGKAPKLIVSDVSERPSGV |
| | | ISTAYMEVSSLTSDDAAIYYCARAR | | PDRFSGSKSGNTASLTISGL |
| | | WDLLPGGRCFDYWGQGTLVTVSSAS | | QAEDEADYYCSSYAGSYTFV |
| | | TKGPSVFPLAPSSKSTSGGTAALGC | | FGGGTKVTVLGQPKAAPSVT |
| | | LVKDYFPEPVTVSWNSGALTSGVHT | | LFPPSSEELQANKATLVCLI |
| | | FPAVLQSSGLYSLSSVVTVPSSSLG | | SDFYPGAVTVAWKADSSPVK |
| | | TQTYICNVNHKPSNTKVDKRVEPKS | | AGVETTTPSKQSNNKYAASS |
| | | CDKTHTCPPCPAPELLGGPSVFLFP | | YLSLTPEQWKSHRSYSCQVT |
| | | PKPKDTLMISRTPEVTCVVVDVSHE | | HEGSTVEKTVAPTEC |
| | | DPEVKFNWYVDGVEVHNAKTKPREE | | |
| | | QYNSTYRVVSVLTVLHQDWLNGKEY | | |
| | | KCKVSNKALPAPIEKTISKAKGQPR | | |
| | | EPQVYTLPPSREEMTKNQVSLTCLV | | |
| | | KGFYPSDIAVEWESNGQPENNYKTT | | |
| | | PPVLDSDGSFFLYSKLTVDKSRWQQ | | |
| | | GNVFSCSVMHEALHNHYTQKSLSLS | | |
| | | PGK | | |
| CH51 | 17 | QVQLQESGAGLLKPSETLSLTCAVY | 18 | EIVLTQSPGTLSLSPGERAT |
| | | GGSFSGYYWSWIRQPPGKGLEWIGE | | LSCRASQSVSGSYLAWYQQK |
| | | IIHSGSTNYNPSLKSRVTISVDTSN | | PGQAPRLLIYGASSRATGIP |
| | | NQFSLKLSSVTAADTAVYYCARGRR | | DRFNGSGSGTDFTLTISRLE |
| | | LLWFGDFDYWGQGTLVTVSSASTKG | | PEDFAVYYCQQYGSSPAFGQ |
| | | PSVFPLAPSSKSTSGGTAALGCLVK | | GTKVEIKRTVAAPSVFIFPP |
| | | DYFPEPVTVSWNSGALTSGVHTFPA | | SDEQLKSGTASVVCLLNNFY |
| | | VLQSSGLYSLSSVVTVPSSSLGTQT | | PREAKVQWKVDNALQSGNSQ |
| | | YICNVNHKPSNTKVDKRVEPKSCDK | | ESVTEQDSKDSTYSLSSTLT |
| | | THTCPPCPAPELLGGPSVFLFPPKP | | LSKADYEKHKVYACEVTHQG |
| | | KDTLMISRTPEVTCVVVDVSHEDPE | | LSSPVTKSFNRGEC |
| | | VKFNWYVDGVEVHNAKTKPREEQYN | | |
| | | STYRVVSVLTVLHQDWLNGKEYKCK | | |
| | | VSNKALPAPIEKTISKAKGQPREPQ | | |
| | | VYTLPPSREEMTKNQVSLTCLVKGF | | |
| | | YPSDIAVEWESNGQPENNYKTTPPV | | |
| | | LDSDGSFFLYSKLTVDKSRWQQGNV | | |
| | | FSCSVMHEALHNHYTQKSLSLSPGK | | |
| CH52 | 19 | QVQLVQSGAEVKKPGASVKVSCKAS | 20 | EIVLTQSPGTLSLSPGERAT |
| | | GYTFTGYYMHWVRQAPGQGLEWMGG | | LSCRASQSISINYLAWYQQR |
| | | INSNSGGTNFAQKFQGRVTMTRDTS | | PGQAPRLLISGASSRATGIP |
| | | ISTAYMELSRLRSDDTAVYYCASTY | | DRFSGSGSGTDFTLTISRLE |
| | | SSTWFRFDYWGQGTLVTVSSASTKG | | PEDFAVYYCQQYGSSPPYTF |
| | | PSVFPLAPSSKSTSGGTAALGCLVK | | GQGTKLEIKRTVAAPSVFIF |
| | | DYFPEPVTVSWNSGALTSGVHTFPA | | PPSDEQLKSGTASVVCLLNN |
| | | VLQSSGLYSLSSVVTVPSSSLGTQT | | FYPREAKVQWKVDNALQSGN |
| | | YICNVNHKPSNTKVDKRVEPKSCDK | | SQESVTEQDSKDSTYSLSST |
| | | THTCPPCPAPELLGGPSVFLFPPKP | | LTLSKADYEKHKVYACEVTH |
| | | KDTLMISRTPEVTCVVVDVSHEDPE | | QGLSSPVTKSFNRGEC |
| | | VKFNWYVDGVEVHNAKTKPREEQYN | | |
| | | STYRVVSVLTVLHQDWLNGKEYKCK | | |
| | | VSNKALPAPIEKTISKAKGQPREPQ | | |
| | | VYTLPPSREEMTKNQVSLTCLVKGF | | |
| | | YPSDIAVEWESNGQPENNYKTTPPV | | |
| | | LDSDGSFFLYSKLTVDKSRWQQGNV | | |
| | | FSCSVMHEALHNHYTQKSLSLSPGK | | |
| CH53 | 21 | QVQLVQSGAEVKKPGASVKVSCKAS | 22 | QSALTQPRSVSGSPGQSVTI |
| | | GYTFIGYYMHWVRQAPGQGLEWMGW | | SCTGTSSDVGGYNYVSWCQQ |
| | | INPNSGGTNYAQKFQGRVTMTRDTS | | HPGKAPQLMIYDVSKRPSGV |
| | | IRTVYMELSRLRFDDTAMYYCARAP | | PDRFSGSKSGNMASLTISGL |
| | | SLVVGGGRLVDYWGQGSQVTVSSAS | | QAEDEGDYYCCSYAGNYTLV |
| | | TKGPSVFPLAPSSKSTSGGTAALGC | | FGGGTRLTVLGQPKAAPSVT |
| | | LVKDYFPEPVTVSWNSGALTSGVHT | | LFPPSSEELQANKATLVCLI |
| | | FPAVLQSSGLYSLSSVVTVPSSSLG | | SDFYPGAVTVAWKADSSPVK |
| | | TQTYICNVNHKPSNTKVDKRVEPKS | | AGVETTTPSKQSNNKYAASS |
| | | CDKTHTCPPCPAPELLGGPSVFLFP | | YLSLTPEQWKSHRSYSCQVT |
| | | PKPKDTLMISRTPEVTCVVVDVSHE | | HEGSTVEKTVAPTEC |
| | | DPEVKFNWYVDGVEVHNAKTKPREE | | |
| | | QYNSTYRVVSVLTVLHQDWLNGKEY | | |
| | | KCKVSNKALPAPIEKTISKAKGQPR | | |
| | | EPQVYTLPPSREEMTKNQVSLTCLV | | |
| | | KGFYPSDIAVEWESNGQPENNYKTT | | |
| | | PPVLDSDGSFFLYSKLTVDKSRWQQ | | |
| | | GNVFSCSVMHEALHNHYTQKSLSLS | | |
| | | PGK | | |
| CH54 | 23 | QVQLVQSGPEVKKPGTSMKISCKAS | 24 | DIQMTQSPSSLSASVGDRVT |
| | | GFTFTRSTMQWVRQARGQRLEWIGW | | ITCRASQSIINYLNWYQQKP |
| | | IVVGSGNTNYAQKFQERVTITRDMS | | GRAPKLLIYAASSLLSGVPS |
| | | TSTAYMELSSLRSEDTAVYYCAAAP | | RFSGSGSGTDFTLTISSLQP |
| | | VGPTSSDYWGQGTLVTVSSASTKGP | | EDFATYYCQQSYSTPYTFGQ |
| | | SVFPLAPSSKSTSGGTAALGCLVKD | | GTKLEIKGQPKAAPSVTLFP |
| | | YFPEPVTVSWNSGALTSGVHTFPAV | | PSSEELQANKATLVCLISDF |
| | | LQSSGLYSLSSVVTVPSSSLGTQTY | | YPGAVTVAWKADSSPVKAGV |
| | | ICNVNHKPSNTKVDKRVEPKSCDKT | | ETTTPSKQSNNKYAASSYLS |
| | | HTCPPCPAPELLGGPSVFLFPPKPK | | LTPEQWKSHRSYSCQVTHEG |
| | | DTLMISRTPEVTCVVVDVSHEDPEV | | STVEKTVAPTEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| CH55 | 25 | QVQLVQSGAEVKKPGASVKVSCKAS | 26 | EIVMTQSPATLSVSPGERAT |
| | | GYIFISYFMHWVRQAPGQGLEWMGI | | LSCRASQSISSNLAWYQQKP |
| | | INPSSGDTRYAQKFQGRVTMTRDTS | | GQAPRLLIYGASTRATGTPA |
| | | TNTVYMELSSLRSDDTAVYYCARRP | | RFSGSGSGTEFTLTISSLQS |
| | | GGLERHNWLDPWGQGTLVTVSSAST | | EDFASYYCQQYNNWPAITFG |
| | | KGPSVFPLAPSSKSTSGGTAALGCL | | QGTRLEIKRTVAAPSVFIFP |
| | | VKDYFPEPVTVSWNSGALTSGVHTF | | PSDEQLKSGTASVVCLLNNF |
| | | PAVLQSSGLYSLSSVVTVPSSSLGT | | YPREAKVQWKVDNALQSGNS |
| | | QTYICNVNHKPSNTKVDKRVEPKSC | | QESVTEQDSKDSTYSLSSTL |
| | | DKTHTCPPCPAPELLGGPSVFLFPP | | TLSKADYEKHKVYACEVTHQ |
| | | KPKDTLMISRTPEVTCVVVDVSHED | | GLSSPVTKSFNRGEC |
| | | PEVKFNWYVDGVEVHNAKTKPREEQ | | |
| | | YNSTYRVVSVLTVLHQDWLNGKEYK | | |
| | | CKVSNKALPAPIEKTISKAKGQPRE | | |
| | | PQVYTLPPSREEMTKNQVSLTCLVK | | |
| | | GFYPSDIAVEWESNGQPENNYKTTP | | |
| | | PVLDSDGSFFLYSKLTVDKSRWQQG | | |
| | | NVFSCSVMHEALHNHYTQKSLSLSP | | |
| | | GK | | |
| CH57 | 27 | QVQLVQSGAEVKKPGASVKVSCKAS | 28 | DIQMTQSPSSLSASVGDRVT |
| | | GYTFTGYYMHWVRQAPGQGLEWMGR | | ITCRASQSIIKYLNWYQQRP |
| | | INPNTGGTNYAQKFQGRVIMTRDTS | | GKAPKLLIYATSTLQSGVPA |
| | | IKTTYMELSSLRSDDMAVYYCARSA | | RFSGSGSGTDFTLTISSLQP |
| | | TGYYGMDAWGQGTTVTVSSASTKGP | | EDFATYYCQQSYSTLWTFGQ |
| | | SVFPLAPSSKSTSGGTAALGCLVKD | | GTKVEIERTVAAPSVFIFPP |
| | | YFPEPVTVSWNSGALTSGVHTFPAV | | SDEQLKSGTASVVCLLNNFY |
| | | LQSSGLYSLSSVVTVPSSSLGTQTY | | PREAKVQWKVDNALQSGNSQ |
| | | ICNVNHKPSNTKVDKRVEPKSCDKT | | ESVTEQDSKDSTYSLSSTLT |
| | | HTCPPCPAPELLGGPSVFLFPPKPK | | LSKADYEKHKVYACEVTHQG |
| | | DTLMISRTPEVTCVVVDVSHEDPEV | | LSSPVTKSFNRGEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| CH77 | 29 | QVQLVQSGAEVKKPGASVKVSCKAS | 30 | DIVMTQSPDSLALSLGERAT |
| | | GYTFIAYYMHWVRQAPGQGLEWMGW | | INCKSSQSVLYNSNNKNYLA |
| | | INPNSGGTNYAQKFQGRVTMTRDTS | | WYQQKPGQPPKLLIYWASTR |
| | | ISTAYMELSRLRFDDTAVYYCGRFS | | ESGVPDRFSGSGSGTDFTLT |
| | | GNYFLYHGMDVWGQGTTVTVSSAST | | ISSLQAEDVAVYYCQQYYSN |
| | | KGPSVFPLAPSSKSTSGGTAALGCL | | LTFGGGTKVEIKRTVAAPSV |
| | | VKDYFPEPVTVSWNSGALTSGVHTF | | FIFPPSDEQLKSGTASVVCL |
| | | PAVLQSSGLYSLSSVVTVPSSSLGT | | LNNFYPREAKVQWKVDNALQ |
| | | QTYICNVNHKPSNTKVDKRVEPKSC | | SGNSQESVTEQDSKDSTYSL |
| | | DKTHTCPPCPAPELLGGPSVFLFPP | | SSTLTLSKADYEKHKVYACE |
| | | KPKDTLMISRTPEVTCVVVDVSHED | | VTHQGLSSPVTKSFNRGEC |
| | | PEVKFNWYVDGVEVHNAKTKPREEQ | | |
| | | YNSTYRVVSVLTVLHQDWLNGKEYK | | |
| | | CKVSNKALPAPIEKTISKAKGQPRE | | |
| | | PQVYTLPPSREEMTKNQVSLTCLVK | | |
| | | GFYPSDIAVEWESNGQPENNYKTTP | | |
| | | PVLDSDGSFFLYSKLTVDKSRWQQG | | |
| | | NVFSCSVMHEALHNHYTQKSLSLSP | | |
| | | GK | | |
| CH78 | 31 | QVQLVQSGAEVKKPGASVKVSCKAS | 32 | EIVLTQSPATLSLSPGERAT |
| | | GYTFTGYYMHWVRQAPGQGLEWMGW | | LSCRASQSVSSYLAWYQQKP |
| | | INPNSGGTNYAQKFQGRVTMTRDTS | | GQAPRLLIYDASKRATGIPA |
| | | ISTAYMELSRLRSDDTAVYFCARVW | | RFSVSGSGTDFTLTISSLEP |
| | | VYYYDSSGYSYPFDYWGQGTLVTVS | | EDFAVYYCQQRSHWTWTFGQ |
| | | SASTKGPSVFPLAPSSKSTSGGTAA | | GTRVEIKRTVAAPSVFIFPP |
| | | LGCLVKDYFPEPVTVSWNSGALTSG | | SDEQLKSGTASVVCLLNNFY |
| | | VHTFPAVLQSSGLYSLSSVVTVPSS | | PREAKVQWKVDNALQSGNSQ |
| | | SLGTQTYICNVNHKPSNTKVDKRVE | | ESVTEQDSKDSTYSLSSTLT |
| | | PKSCDKTHTCPPCPAPELLGGPSVF | | LSKADYEKHKVYACEVTHQG |
| | | LFPPKPKDTLMISRTPEVTCVVVDV | | LSSPVTKSFNRGEC |
| | | SHEDPEVKFNWYVDGVEVHNAKTKP | | |
| | | REEQYNSTYRVVSVLTVLHQDWLNG | | |
| | | KEYKCKVSNKALPAPIEKTISKAKG | | |
| | | QPREPQVYTLPPSREEMTKNQVSLT | | |
| | | CLVKGFYPSDIAVEWESNGQPENNY | | |
| | | KTTPPVLDSDGSFFLYSKLTVDKSR | | |
| | | WQQGNVFSCSVMHEALHNHYTQKSL | | |
| | | SLSPGK | | |
| CH80 | 33 | QVQLVQSGAEVKKPGASVKVSCKAS | 34 | DIQLTQSPSSLSASVGDRVT |
| | | GYTFIGYYIHWVRQAPGQGLEWMGW | | ITCRASQGISNYLAWYQQKP |
| | | INPNSGGTNYAQKFQGRVTMTRDTS | | GKIPRLLIYAASTLQSGVPS |
| | | ITTAYMELTRLRSDDTAVYYCARGG | | RFSGSGSGTDFTLTISSLQP |
| | | LPGTGTAYWGQGTLVTVSSASTKGP | | EDVATYYCQKFNSVPPLTFG |
| | | SVFPLAPSSKSTSGGTAALGCLVKD | | GGTKVEIKRTVAAPSVFIFP |
| | | YFPEPVTVSWNSGALTSGVHTFPAV | | PSDEQLKSGTASVVCLLNNF |
| | | LQSSGLYSLSSVVTVPSSSLGTQTY | | YPREAKVQWKVDNALQSGNS |
| | | ICNVNHKPSNTKVDKRVEPKSCDKT | | QESVTEQDSKDSTYSLSSTL |
| | | HTCPPCPAPELLGGPSVFLFPPKPK | | TLSKADYEKHKVYACEVTHQ |
| | | DTLMISRTPEVTCVVVDVSHEDPEV | | GLSSPVTKSFNRGEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| CH81 | 35 | QVQLVQSGAEVKKPGASVKVSCKAS | 36 | DIQMTQSPSSLSASVGDRVT |
| | | GYTFTSYDINWVRQATGQGLECMGW | | ITCRASQSISNYLNWYQQKP |
| | | MNPNSGNTGYAQKFQGRVTMTRNTS | | RKAPKLLIYAASSLQSGVPS |
| | | ISTAYMELSSLRSDDTAVYYCARGL | | RFSGSGSGADFTLTISSLQP |
| | | RTYYYGSGYYRPLGYWGQGTLVTVS | | EDFATYYCQQSYSTLQTFGQ |
| | | SASTKGPSVFPLAPSSKSTSGGTAA | | GTKLEIKRTVAAPSVFIFPP |
| | | LGCLVKDYFPEPVTVSWNSGALTSG | | SDEQLKSGTASVVCLLNNFY |
| | | VHTFPAVLQSSGLYSLSSVVTVPSS | | PREAKVQWKVDNALQSGNSQ |
| | | SLGTQTYICNVNHKPSNTKVDKRVE | | ESVTEQDSKDSTYSLSSTLT |
| | | PKSCDKTHTCPPCPAPELLGGPSVF | | LSKADYEKHKVYACEVTHQG |
| | | LFPPKPKDTLMISRTPEVTCVVVDV | | LSSPVTKSFNRGEC |
| | | SHEDPEVKFNWYVDGVEVHNAKTKP | | |
| | | REEQYNSTYRVVSVLTVLHQDWLNG | | |
| | | KEYKCKVSNKALPAPIEKTISKAKG | | |
| | | QPREPQVYTLPPSREEMTKNQVSLT | | |
| | | CLVKGFYPSDIAVEWESNGQPENNY | | |
| | | KTTPPVLDSDGSFFLYSKLTVDKSR | | |
| | | WQQGNVFSCSVMHEALHNHYTQKSL | | |
| | | SLSPGK | | |
| CH89 | 37 | EVQLVESGAEVKKPGASVKVSCKAS | 38 | DIVLTQSPSSLSASVGDRVT |
| | | GYTFTGYYMHWVRQAPGQGLEWMGW | | ITCRTSQSISNYLNWYQQKP |
| | | INPNSGGTNYAPKFQGRVTMTRDTS | | GKAPKLLIYAASSLQSGVPS |
| | | ISTAYMELTGLRSDDTAVYYCARSI | | RFSGSGSGTDFTLTVSSLQP |
| | | TLIVNFAYWGQGTLVTVSSASTKGP | | EDFATYYCQQTYNTPVTFGG |
| | | SVFPLAPSSKSTSGGTAALGCLVKD | | GTKVEIKRTVAAPSVFIFPP |
| | | YFPEPVTVSWNSGALTSGVHTFPAV | | SDEQLKSGTASVVCLLNNFY |
| | | LQSSGLYSLSSVVTVPSSSLGTQTY | | PREAKVQWKVDNALQSGNSQ |
| | | ICNVNHKPSNTKVDKRVEPKSCDKT | | ESVTEQDSKDSTYSLSSTLT |
| | | HTCPPCPAPELLGGPSVFLFPPKPK | | LSKADYEKHKVYACEVTHQG |
| | | DTLMISRTPEVTCVVVDVSHEDPEV | | LSSPVTKSFNRGEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| CH90 | 39 | QVQLVQSGAEVKKPGASVKVSCKAS | 40 | EIVLTQSPSSLSASVGDRVT |
| | | GYTFISYYMHWVRQAPGQGLEWMGI | | ITCRASQGISNSLAWYQQKP |
| | | INSSGGSTNYAQKFQGRVTMTRDTS | | GKAPKLLLYAASRLESGVPS |
| | | TSTVYMELSSLRSEDTAVYYCARGG | | RFSGSGSGTDYTLTISSLQP |
| | | ITLVRGVIYYWGQGTLVTVSSASTK | | EDFATYYCQQYYSTLPTFGQ |
| | | GPSVFPLAPSSKSTSGGTAALGCLV | | GTKVEIKRTVAAPSVFIFPP |
| | | KDYFPEPVTVSWNSGALTSGVHTFP | | SDEQLKSGTASVVCLLNNFY |
| | | AVLQSSGLYSLSSVVTVPSSSLGTQ | | PREAKVQWKVDNALQSGNSQ |
| | | TYICNVNHKPSNTKVDKRVEPKSCD | | ESVTEQDSKDSTYSLSSTLT |
| | | KTHTCPPCPAPELLGGPSVFLFPPK | | LSKADYEKHKVYACEVTHQG |
| | | PKDTLMISRTPEVTCVVVDVSHEDP | | LSSPVTKSFNRGEC |
| | | EVKFNWYVDGVEVHNAKTKPREEQY | | |
| | | NSTYRVVSVLTVLHQDWLNGKEYKC | | |
| | | KVSNKALPAPIEKTISKAKGQPREP | | |
| | | QVYTLPPSREEMTKNQVSLTCLVKG | | |
| | | FYPSDIAVEWESNGQPENNYKTTPP | | |
| | | VLDSDGSFFLYSKLTVDKSRWQQGN | | |
| | | VFSCSVMHEALHNHYTQKSLSLSPG | | |
| | | K | | |
| CH91 | 41 | QVQLQESGPGLVKPSQTLSLTCTVS | 42 | QSALTQPRSVSGSPGQSVTI |
| | | GGSISSGGYYWSWIRQHPGKGLEWI | | SCTGTSSDVGGYDYVSWYQQ |
| | | GNIYYSGSTYYNPSLKSRITISLDT | | HPGKAPKLMISDVSKRPSGV |
| | | SKNQFSLKLSSVTAADTAVYYCART | | PDRFSGSKSGNTASLTISGL |
| | | SRTTVLRNAFDIWGHGTMVTVSSAS | | QPEDEADYYCCSYAGTYTYW |
| | | TKGPSVFPLAPSSKSTSGGTAALGC | | VFGGGTKLTVLGQPKAAPSV |
| | | LVKDYFPEPVTVSWNSGALTSGVHT | | TLFPPSSEELQANKATLVCL |
| | | FPAVLQSSGLYSLSSVVTVPSSSLG | | ISDFYPGAVTVAWKADSSPV |
| | | TQTYICNVNHKPSNTKVDKRVEPKS | | KAGVETTTPSKQSNNKYAAS |
| | | CDKTHTCPPCPAPELLGGPSVFLFP | | SYLSLTPEQWKSHRSYSCQV |
| | | PKPKDTLMISRTPEVTCVVVDVSHE | | THEGSTVEKTVAPTEC |
| | | DPEVKFNWYVDGVEVHNAKTKPREE | | |
| | | QYNSTYRVVSVLTVLHQDWLNGKEY | | |
| | | KCKVSNKALPAPIEKTISKAKGQPR | | |
| | | EPQVYTLPPSREEMTKNQVSLTCLV | | |
| | | KGFYPSDIAVEWESNGQPENNYKTT | | |
| | | PPVLDSDGSFFLYSKLTVDKSRWQQ | | |
| | | GNVFSCSVMHEALHNHYTQKSLSLS | | |
| | | PGK | | |
| CH92 | 43 | QVQLVQSGAEVKKPGASVKVSCKAS | 44 | DIQMTQSPSSVSASVGDRVT |
| | | GYTFTGYYIHWVRQAPGQGLEWMGW | | ITCRASQGISTWLAWYQQKP |
| | | INPNSGGTNYAQKFQGRVTMTRDTS | | GKAPKALIYAATRLQSGVPS |
| | | ISAAYMELSSLLFDDTAVYYCARVP | | RFSGSGSGTDFTLTISSLQP |
| | | HIVVVVAAANANFDYWGQGTLVTVA | | EDFAIYYCQQANSFPITFGQ |
| | | SASTKGPSVFPLAPSSKSTSGGTAA | | GTRLEIKRTVAAPSVFIFPP |
| | | LGCLVKDYFPEPVTVSWNSGALTSG | | SDEQLKSGTASVVCLLNNFY |
| | | VHTFPAVLQSSGLYSLSSVVTVPSS | | PREAKVQWKVDNALQSGNSQ |
| | | SLGTQTYICNVNHKPSNTKVDKRVE | | ESVTEQDSKDSTYSLSSTLT |
| | | PKSCDKTHTCPPCPAPELLGGPSVF | | LSKADYEKHKVYACEVTHQG |
| | | LFPPKPKDTLMISRTPEVTCVVVDV | | LSSPVTKSFNRGEC |
| | | SHEDPEVKFNWYVDGVEVHNAKTKP | | |
| | | REEQYNSTYRVVSVLTVLHQDWLNG | | |
| | | KEYKCKVSNKALPAPIEKTISKAKG | | |
| | | QPREPQVYTLPPSREEMTKNQVSLT | | |
| | | CLVKGFYPSDIAVEWESNGQPENNY | | |
| | | KTTPPVLDSDGSFFLYSKLTVDKSR | | |
| | | WQQGNVFSCSVMHEALHNHYTQKSL | | |
| | | SLSPGK | | |
| CH94 | 45 | VQLVESGAEVKKPGASVQVSCKASG | 46 | EIVLTQSPSSLSASVGDRVT |
| | | YIFKSYYVHWVRQAPGQGLEWMGII | | FTCRASQSMSSCLNWYQQKP |
| | | NPSGGTASYAQKFQGRVTMTRDTST | | GKAPKLLIYAASSLQSGVPS |
| | | STVYMELSSLRSEDTAVYYCARDNG | | RFSGSGSGTDFTLTISSLQP |
| | | IVGYSGSRGYYYYYGMDVWGQGTTV | | EDFATYYCQQSYSTPYTFGQ |
| | | TVSSASTKGPSVFPLAPSSKSTSGG | | GTKLEINRTVAAPSVFIFPP |
| | | TAALGCLVKDYFPEPVTVSWNSGAL | | SDEQLKSGTASVVCLLNNFY |
| | | TSGVHTFPAVLQSSGLYSLSSVVTV | | PREAKVQWKVDNALQSGNSQ |
| | | PSSSLGTQTYICNVNHKPSNTKVDK | | ESVTEQDSKDSTYSLSSTLT |
| | | RVEPKSCDKTHTCPPCPAPELLGGP | | LSKADYEKHKVYACEVTHQG |
| | | SVFLFPPKPKDTLMISRTPEVTCVV | | LSSPVTKSFNRGEC |
| | | VDVSHEDPEVKFNWYVDGVEVHNAK | | |
| | | TKPREEQYNSTYRVVSVLTVLHQDW | | |
| | | LNGKEYKCKVSNKALPAPIEKTISK | | |
| | | AKGQPREPQVYTLPPSREEMTKNQV | | |
| | | SLTCLVKGFYPSDIAVEWESNGQPE | | |
| | | NNYKTTPPVLDSDGSFFLYSKLTVD | | |
| | | KSRWQQGNVFSCSVMHEALHNHYTQ | | |
| | | KSLSLSPGK | | |
| DH677.3 | 47 | QVQLVQSGAEVQKPGASVKVSCKAS | 48 | DIQLTQSPSSLSASVGDSVT |
| | | GYTFASYDINWVRQATGQGLEWMGW | | ITCRASQGFGNYLAWYQQRP |
| | | MNPKTGNTGYAQKFQGRVTLTRNTS | | GKVPEVLIYAATTLQSGVPS |
| | | ISTAYMELTSLRSEDTAVYYCATYR | | RFSGSGSGTDFTLTISSLQP |
| | | IIAAVGYRYFQYWGQGTLVTVSSAS | | EDVATYYCQKYNSAPFTFGQ |
| | | TKGPSVFPLAPSSKSTSGGTAALGC | | GTRLEIKRTVAAPSVFIFPP |
| | | LVKDYFPEPVTVSWNSGALTSGVHT | | SDEQLKSGTASVVCLLNNFY |
| | | FPAVLQSSGLYSLSSVVTVPSSSLG | | PREAKVQWKVDNALQSGNSQ |
| | | TQTYICNVNHKPSNTKVDKRVEPKS | | ESVTEQDSKDSTYSLSSTLT |
| | | CDKTHTCPPCPAPELLGGPSVFLFP | | LSKADYEKHKVYACEVTHQG |
| | | PKPKDTLMISRTPEVTCVVVDVSHE | | LSSPVTKSFNRGEC |
| | | DPEVKFNWYVDGVEVHNAKTKPREE | | |
| | | QYNSTYRVVSVLTVLHQDWLNGKEY | | |
| | | KCKVSNKALPAPIEKTISKAKGQPR | | |
| | | EPQVYTLPPSREEMTKNQVSLTCLV | | |
| | | KGFYPSDIAVEWESNGQPENNYKTT | | |
| | | PPVLDSDGSFFLYSKLTVDKSRWQQ | | |
| | | GNVFSCSVMHEALHNHYTQKSLSLS | | |
| | | PGK | | |
| JR4 | 49 | EVQLVESGPGLVKPLETLSLTCAVP | 50 | QSVLTQPPSVSAAPGQKVTI |
| | | GGSIRRNYWSWIRQPPGKGLEWIGH | | SCSGSSSNIGRSYVSWYQQV |
| | | SYGSGGSTNYNPSLESRVTLSVDTS | | PGAAPKLLIYDTNKRPSGVS |
| | | KNLFSLKLTSVTAADTAVYYCARTV | | DRFSGSKSGSSASLAITGLQ |
| | | WYYTSGTHYFDHWGQGVLVTVSSAS | | TGDEADYYCGAWDGSLNVHI |
| | | TKGPSVFPLAPSSRSTSESTAALGC | | FGSGTKLTVLGQPKASPLVT |
| | | LVKDYFPEPVTVSWNSGSLTSGVHT | | LFPPSSEELQANKATLVCLI |
| | | FPAVLQSSGLYSLSSVVTVPSSSLG | | SDFYPGVVKVAWKADGNSVN |
| | | TQTYVCNVNHKPSNTKVDKRVEIKT | | TGVETTTPSKQSNNKYAASS |
| | | CGGGSKPPTCPPCPAPELLGGPSVF | | YLSLTSDQWKSHKSYSCQVT |
| | | LFPPKPKDTLMISRTPEVTCVVVDV | | HEGSTVEKTVAPTEC |
| | | SQEDPDVKFNWYVNGAEVHHAQTKP | | |
| | | RETQYNSTYRVVSVLTVTHQDWLNG | | |
| | | KEYTCKVSNKALPAPIQKTISKDKG | | |
| | | QPREPQVYTLPPSREELTKNQVSLT | | |
| | | CLVKGFYPSDIVVEWESSGQPENTY | | |
| | | KTTPPVLDSDGSYFLYSKLTVDKSR | | |
| | | WQQGNVFSCSVMHEALHNHYTQKSL | | |
| | | SVSPGK | | |
| N12-i3 | 51 | QVQLVQSGAEVKKPGSSVRVSCKAS | 52 | EIVLTQSPGTLSMSPGERAT |
| | | GGSFSRYTVNWVRQAPGQGLEWMAR | | LSCRASRTVSSSNLAWYQQK |
| | | FIPIFNMPDYAPKFQGRITITADES | | PGQAPRLLIYDVSSRATGIP |
| | | TSTAYLELSSLTSDDTAVYYCASRQ | | DRFSGRGSGTDFTLTISRLE |
| | | HHEYFQEWGQGTLVTVSSASTKGPS | | PEDFAVYYCQQYGTSPLTFG |
| | | VFPLAPSSKSTSGGTAALGCLVKDY | | GGTKVEIKRTVAAPSVFIFP |
| | | FPEPVTVSWNSGALTSGVHTFPAVL | | PSDEQLKSGTASVVCLLNNF |
| | | QSSGLYSLSSVVTVPSSSLGTQTYI | | YPREAKVQWKVDNALQSGNS |
| | | CNVNHKPSNTKVDKRVEPKSCDKTH | | QESVTEQDSKDSTYSLSSTL |
| | | TCPPCPAPELLGGPSVFLFPPKPKD | | TLSKADYEKHKVYACEVTHQ |
| | | TLMISRTPEVTCVVVDVSHEDPEVK | | GLSSPVTKSFNRGEC |
| | | FNWYVDGVEVHNAKTKPREEQYNST | | |
| | | YRVVSVLTVLHQDWLNGKEYKCKVS | | |
| | | NKALPAPIEKTISKAKGQPREPQVY | | |
| | | TLPPSREEMTKNQVSLTCLVKGFYP | | |
| | | SDIAVEWESNGQPENNYKTTPPVLD | | |
| | | SDGSFFLYSKLTVDKSRWQQGNVFS | | |
| | | CSVMHEALHNHYTQKSLSLSPGK | | |
| N5-i5 | 53 | EVQLVESGGGLVQPGGSLRLSCAAS | 54 | QSALTQPASVSGSPGQSITI |
| | | GFTFSTYAMSWVRQAPGKGLEWVSS | | SCTGTSSDVGSYNFVSWYQQ |
| | | INNSGRNTFSADSVKGRFTISRDNS | | HPGKAPKLMIYEVSERPSGI |
| | | KNTLFLVMNSLRAEDTAVYYCAKDL | | SNRFSGSKSGNTASLTISGL |
| | | RLGGGSDYWGQGTLVTVSSASTKGP | | QAEDEADYYCSSYAGSTTFR |
| | | SVFPLAPSSKSTSGGTAALGCLVKD | | VFGGGTKLTVRGQPKAAPSV |
| | | YFPEPVTVSWNSGALTSGVHTFPAV | | TLFPPSSEELQANKATLVCL |
| | | LQSSGLYSLSSVVTVPSSSLGTQTY | | ISDFYPGAVTVAWKADSSPV |
| | | ICNVNHKPSNTKVDKRVEPKSCDKT | | KAGVETTTPSKQSNNKYAAS |
| | | HTCPPCPAPELLGGPSVFLFPPKPK | | SYLSLTPEQWKSHRSYSCQV |
| | | DTLMISRTPEVTCVVVDVSHEDPEV | | THEGSTVEKTVAPTEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| N60-i3 | 55 | TGVHSEVQLVESGPGLVKPSQTLSL | 56 | QSVLTQPASVSGSPGQSITI |
| | | TCTVSGASISSGGYFWSWIRQHPGK | | SCTGTSSDVGGYKYVSWYQQ |
| | | GLEWIGNIYYIGNTYYNPSLKSRLT | | HPDKAPKLMIYEVSNRPSGV |
| | | ISVDTTQNQFSLKLTSVTAADTAVY | | SNRFSGSKSGNTASLTISGL |
| | | YCARVPRLRGGNYFDSWGQGTLVTV | | QAEDEADYYCSSYTSSSTWV |
| | | SSASTKGPSVFPLAPSSKSTSGGTA | | FGGGTKLTVLGQPKAAPSVT |
| | | ALGCLVKDYFPEPVTVSWNSGALTS | | LFPPSSEELQANKATLVCLI |
| | | GVHTFPAVLQSSGLYSLSSVVTVPS | | SDFYPGAVTVAWKADSSPVK |
| | | SSLGTQTYICNVNHKPSNTKVDKRV | | AGVETTTPSKQSNNKYAASS |
| | | EPKSCDKTHTCPPCPAPELLGGPSV | | YLSLTPEQWKSHRSYSCQVT |
| | | FLFPPKPKDTLMISRTPEVTCVVVD | | HEGSTVEKTVAPTE |
| | | VSHEDPEVKFNWYVDGVEVHNAKTK | | |
| | | PREEQYNSTYRVVSVLTVLHQDWLN | | |
| | | GKEYKCKVSNKALPAPIEKTISKAK | | |
| | | GQPREPQVYTLPPSREEMTKNQVSL | | |
| | | TCLVKGFYPSDIAVEWESNGQPENN | | |
| | | YKTTPPVLDSDGSFFLYSKLTVDKS | | |
| | | RWQQGNVFSCSVMHEALHNHYTQKS | | |
| | | LSLSPGK | | |

In certain embodiments, the non-neutralizing antibody in the Ab-CD4 conjugate disclosed herein is an intact Cluster A antibody selected from 2.2c, A32, C11, CH20, CH29, CH38, CH40, CH49, CH51, CH52, CH53, CH54, CH55, CH57, CH77, CH78, CH80, CH81, CH89, CH90, CH91, CH92, CH94, DH677.3, JR4, N12-i3, N5-i5, and N60-i3. In certain embodiments, the Cluster A antibody is A32, and in certain embodiments, the Cluster A antibody is N5-i5.

CoRBS, unlike the Cluster A binding site, is localized at the surface of the Env trimer, mapping to the outer domain of gp120, proximal to the CD4 binding site. Unlike the Cluster A region, which maps to the interior of the HIV Env trimer, the CoRBS is used as a second attachment point by HIV virions to specifically engage the coreceptor on the cell surface. CoRBS antibodies, which are unable to mediate potent ADCC or ADCP activity on their own, have nonetheless been shown to facilitate engagement of Cluster A antibodies, potentiating their ADCC and/or ADCP activity.

In certain embodiments, the antibody in the Ab-CD4 conjugate disclosed herein is an intact CoRBS antibody selected from 17b (having a heavy chain sequence of SEQ ID NO: 57 and a light chain sequence of SEQ ID NO: 58 or having the 6 CDRs of SEQ ID NOs: 57 and 58), 412d (having a heavy chain sequence of SEQ ID NO: 59 and a light chain sequence of SEQ ID NO: 60 or having the 6 CDRs of SEQ ID NOs: 59 and 60), 48d (having a heavy chain sequence of SEQ ID NO: 61 and a light chain sequence of SEQ ID NO: 62 or having the 6 CDRs of SEQ ID NOs: 61 and 62), E51 (having a heavy chain sequence of SEQ ID NO: 63 and a light chain sequence of SEQ ID NO: 64 or having the 6 CDRs of SEQ ID NOs: 63 and 64), N12-i2 (having a heavy chain sequence of SEQ ID NO: 65 and a light chain sequence of SEQ ID NO: 66 or having the 6 CDRs of SEQ ID NOs: 65 and 66), and X5 (having a heavy chain sequence of SEQ ID NO: 67 and a light chain sequence of SEQ ID NO: 68 or having the 6 CDRs of SEQ ID NOs: 67 and 68), including those disclosed, for example, in U.S. Published Patent Application No 2015/0175678. The heavy and light chain amino acid sequences of exemplary CoRBS antibodies are shown below in Table B. In certain embodiments, the CoRBS antibody is X5, and in certain embodiments, the CoRBS antibody is 17b. The CoRBS antibody 17b recognizes a conserved epitope within the bridging sheet of the CoRBS, while the X5 antibody combines the elements of the highly-conserved bridging sheet of the CoRBS with the elements of the V3 loop stem.

TABLE B
CoRBS Antibody Polypeptide Sequences

| | SEQ | | SEQ | |
|---|---|---|---|---|
| Antibody | ID | Heavy chain amino acid | ID | Light chain amino acid |
| name | NO. | sequence | NO. | sequence |
| 17b | 57 | QVQLLESGAEVKKPGSSVKVSCKAS | 58 | ELELTQSPATLSVSPGERAT |
| | | GDTFIRYSFTWVRQAPGQGLEWMGR | | LSCRASESVSSDLAWYQQKP |
| | | IITILDVAHYAPHLQGRVTITADKS | | GQAPRLLIYGASTRATGVPA |
| | | TSTVYLELRNLRSDDTAVYFCAGVY | | RFSGSGSGAEFTLTISSLQS |
| | | EGEADEGEYDNNGFLKHWGQGTLVT | | EDFAVYYCQQYNNWPPRYTF |
| | | VTSASTKGPSVFPLAPSSKSTSGGT | | GQGTRLEIKRTVAAPSVFIF |
| | | AALGCLVKDYFPQPVTVSWNSGALT | | PPSDEQLKSGTASVVCLLNN |
| | | SGVHTFPAVLQSSGLYSLSSVVTVP | | FYPREAKVQWKVDNALQSGN |
| | | SSSLGTQTYICNVNHKPSNTKVDKK | | SQESVTEQDSKDSTYSLSST |
| | | VEPKSCDKTHTCPPCPAPELLGGPS | | LTLSKADYEKHKVYACEVTH |
| | | VFLFPPKPKDTLMISRTPEVTCVVV | | QGLSSPVTKSFNRGEC |
| | | DVSHEDPEVKFNWYVDGVEVHNAKT | | |
| | | KPREEQYNSTYRVVSVLTVLHQDWL | | |
| | | NGKEYKCKVSNKALPAPIEKTISKA | | |
| | | KGQPREPQVYTLPPSREEMTKNQVS | | |
| | | LTCLVKGFYPSDIAVEWESNGQPEN | | |
| | | NYKTTPPVLDSDGSFFLYSKLTVDK | | |
| | | SRWQQGNVFSCSVMHEALHNHYTQK | | |
| | | SLSLSPGK | | |
| 412d | 59 | EVQLVQSGAEVKKPGSSVKVSCKAS | 60 | DIQMTQSPSTLSASVGDRVT |
| | | GGTFSNYAINWVRQAPGQGLEWMGG | | ITCRASQSISNWLAWYQQKP |
| | | IIPIFNIAHYAQRFQGRVSITADES | | GRAPKLLMYKASSLKSGVPS |
| | | TSTAYMELSSLRSEDTAVFYCASPY | | RFSGSGSGTEFTLTISSLQS |
| | | PNDYNDYAPEEGMSWYFDLWGRGTL | | DDFATYYCQQHDSSPYTFGQ |
| | | VTVSPASTKGPSVFPLAPSSKSTSG | | GTKLEIKRTVAAPSVFIFPP |
| | | GTAALGCLVKDYFPEPVTVSWNSGA | | SDEQLKSGTASVVCLLNNFY |
| | | LTSGVHTFPAVLQSSGLYSLSSVVT | | PREAKVQWKVDNALQSGNSQ |
| | | VPSSSLGTQTYICNVNHKPSNTKVD | | ESVTEQDSKDSTYSLSSTLT |
| | | KKVEPKSCDKTHTCPPCPAPELLGG | | LSKADYEKHKLYACEVTHQG |
| | | PSVFLFPPKPKDTLMISRTPEVTCV | | LSSPVTKSFNRGEC |
| | | VVDVSHEDPEVKFNWYVDGVEVHNA | | |
| | | KTKPREEQYNSTYRVVSVLTVLHQD | | |
| | | WLNGKEYKCKVSNKALPAPIEKTIS | | |
| | | KAKGQPREPQVYTLPPSREEMTKNQ | | |
| | | VSLTCLVKGFYPSDIAVEWESNGQP | | |
| | | ENNYKTTPPVLDSDGSFFLYSKLTV | | |
| | | DKSRWQQGNVFSCSVMHEALHNHYT | | |
| | | QKSLSLSPGK | | |
| 48d | 61 | EVQLVQSGAEVKKPGATVKISCKAS | 62 | DIQMTQSPSSVSASVGDRVT |
| | | GYTFSDFYMYWVRQAPGKGLEWMGL | | ITCRASQDISTWLAWYQQKP |
| | | IDPEDADTMYAEKFRGRVTITADTS | | GKAPKLLIYAASTLQSGVPS |
| | | TDTGYLELSSLRSEDTAVYYCAADP | | RFSGSGSGTDFSLTINSLQP |
| | | WELNAFNVWGQGTLVSVSSASTKGP | | EDFATYYCQQANSFFTFGGG |
| | | SVFPLAPSSKSTSGGTAALGCLVKD | | TKVEIKRTVAAPSVFIFPPS |
| | | YFPEPVTVSWNSGALTSGVHTFPAV | | DEQLKSGTASVVCLLNNFYP |
| | | LQSSGLYSLSSVVTVPSSSLGTQTY | | REAKVQWKVDNALQSGNSQE |
| | | ICNVNHKPSNTKVDKKVEPKSCDKT | | SVTEQDSKDSTYSLSSTLTL |
| | | HTCPPCPAPELLGGPSVFLFPPKPK | | SKADYEKHKLYACEVTHQGL |
| | | DTLMISRTPEVTCVVVDVSHEDPEV | | SSPVTKSFNRGEC |
| | | KFNWYVDGVEVHNAKTKPREEQYNS | | |
| | | TYRVVSVLTVLHQDWLNGKEYKCKV | | |
| | | SNKALPAPIEKTISKAKGQPREPQV | | |
| | | YTLPPSREEMTKNQVSLTCLVKGFY | | |
| | | PSDIAVEWESNGQPENNYKTTPPVL | | |
| | | DSDGSFFLYSKLTVDKSRWQQGNVF | | |
| | | SCSVMHEALHNHYTQKSLSLSPGK | | |
| E51 | 63 | EVQLVQSGAEVNKPGSSVKVSCQAS | 64 | QSILTQPPSVSAAPGQKVTI |
| | | GATLNSHAFSWVRQAPGQGLEWMAG | | SCSGSSSNIGNNDVSWYQQF |
| | | IIPIFGSSHYAQKFRGRVTISADES | | PGTVPKLVIYENNERPSGIP |
| | | TRTVYLHLRGLRSDDTAVYYCASNS | | DRFSGSKSGTSATLGITGLQ |
| | | IAGVAAAGDYADYDGGYYYDMDVWG | | TGDEADYYCGTWDSSLSAVV |
| | | QGTTVTVSSASTKGPSVFPLAPSSK | | FGGGSKVTVLGQPKAAPSVT |
| | | STSGGTAALGCLVKDYFPEPVTVSW | | LFPPSSEELQANKATLVCLI |
| | | NSGALTSGVHTFPAVLQSSGLYSLS | | SDFYPGAVTVAWKADSSPVK |
| | | SVVTVPSSSLGTQTYICNVNHKPSN | | AGVETTTPSKQSNNKYAASS |
| | | TKVDKRVEPKSCDKTHTCPPCPAPE | | YLSLTPEQWKSHRSYSCQVT |
| | | LLGGPSVFLFPPKPKDTLMISRTPE | | HEGSTVEKTVAPTEC |
| | | VTCVVVDVSHEDPEVKFNWYVDGVE | | |
| | | VHNAKTKPREEQYNSTYRVVSVLTV | | |
| | | LHQDWLNGKEYKCKVSNKALPAPIE | | |
| | | KTISKAKGQPREPQVYTLPPSREEM | | |
| | | TKNQVSLTCLVKGFYPSDIAVEWES | | |
| | | NGQPENNYKTTPPVLDSDGSFFLYS | | |
| | | KLTVDKSRWQQGNVFSCSVMHEALH | | |
| | | NHYTQKSLSLSPGK | | |
| N12-i2 | 65 | QVQLVQSGAEVKKPGSSVKVSCRAS | 66 | EIVMMQSPGTLSLSPGERAT |
| | | RGTFSSYGITWVRQAPGQGLEWMGG | | LSCRASQSVSSSYLAWYQQK |
| | | IIPIFDVTNYAQNFQGRVAITTDAE | | PGQAPRLLIYGASSRATGIP |
| | | MSTAYMELRSLKSEDSAVYYCASDS | | DRFSGSGSGTDFTLTISRLE |
| | | RDFSYYEPGTSYSHYYNIMDVWGQG | | PEDFAVYYCQQYGSSPETFG |
| | | TTVTVSSASTKGPSVFPLAPSSKST | | QGTKVEIKRTVAAPSVFIFP |
| | | SGGTAALGCLVKDYFPEPVTVSWNS | | PSDEQLKSGTASVVCLLNNF |
| | | GALTSGVHTFPAVLQSSGLYSLSSV | | YPREAKVQWKVDNALQSGNS |
| | | VTVPSSSLGTQTYICNVNHKPSNTK | | QESVTEQDSKDSTYSLSSTL |
| | | VDKRVEPKSCDKTHTCPPCPAPELL | | TLSKADYEKHKVYACEVTHQ |
| | | GGPSVFLFPPKPKDTLMISRTPEVT | | GLSSPVTKSFNRGEC |
| | | CVVVDVSHEDPEVKFNWYVDGVEVH | | |
| | | NAKTKPREEQYNSTYRVVSVLTVLH | | |
| | | QDWLNGKEYKCKVSNKALPAPIEKT | | |
| | | ISKAKGQPREPQVYTLPPSREEMTK | | |
| | | NQVSLTCLVKGFYPSDIAVEWESNG | | |
| | | QPENNYKTTPPVLDSDGSFFLYSKL | | |
| | | TVDKSRWQQGNVFSCSVMHEALHNH | | |
| | | YTQKSLSLSPGK | | |
| X5 | 67 | EVQLLEQSGAEVKKPGSSVQVSCKA | 68 | ELVLTQSPGTLSLSAGERAT |
| | | SGGTFSMYGFNWVRQAPGHGLEWMG | | LSCRASQSVSSGSLAWYQQK |
| | | GIIPIFGTSNYAQKFRGRVTFTADQ | | PGQAPRLLIYGASTRATGIP |
| | | ATSTAYMELTNLRSDDTAVYYCARD | | DRFSGSGSGTDFTLTIGRLE |
| | | FGPDWEDGDSYDGSGRGFFDFWGQG | | PEDLAVYYCQQYGTSPYTFG |
| | | TLVTVSSASTKGPSVFPLAPSSKST | | QGTKLEIKRTVAAPSVFIFP |
| | | SGGTAALGCLVKDYFPEPVTVSWNS | | PSDEQLKSGTASVVCLLNNF |
| | | GALTSGVHTFPAVLQSSGLYSLSSV | | YPREAKVQWKVDNALQSGNS |
| | | VTVPSSSLGTQTYICNVNHKPSNTK | | QESVTEQDSRDSTYSLGSTL |
| | | VDKKVEPKSCDKTHTCPPCPAPELL | | TLSKADYEKHKVYACEVTHQ |
| | | GGPSVFLFPPKPKDTLMISRTPEVT | | GLSSPVTKSFNRGEC |
| | | CVVVDVSHEDPEVKFNWYVDGVEVH | | |
| | | NAKTKPREEQYNSTYRVVSVLTVLH | | |
| | | QDWLNGKEYKCKVSNKALPAPIEKT | | |
| | | ISKAKGQPREPQVYTLPPSREEMTK | | |
| | | NQVSLTCLVKGFYPSDIAVEWESNG | | |
| | | QPENNYKTTPPVLDSDGSFFLYSKL | | |
| | | TVDKSRWQQGNVFSCSVMHEALHNH | | |
| | | YTQKSLSLSPGK | | |

In various embodiments of the disclosure, the Ab-CD4 conjugate comprises at least one antibody selected from Cluster A antibodies and CoRBS antibodies.

In various embodiments, the antibody of the Ab-CD4 conjugates disclosed herein are full-length antibodies and not, for example, a single chain antibody fragment, Fab fragment, or Fv fragment. The full-length antibodies disclosed herein comprise at least an Fc region and a Fab region. In this way, the Ab-CD4 conjugates comprising an Fc region are able to mediate Fc receptor functions involved in targeting and killing HIV virions and HIV-infected cells.

### Fc Modifications

In some embodiments, an Fc portion of an antibody or Ab-CD4 or Ab-CD4mc conjugate described herein is modified to increase its antibody serum-half life in vivo. In some embodiments, an Fc modified antibody or Ab-CD4 conjugate extends its therapeutic and/or protective activity. Such modifications to the Fc region can circumvent the need for frequent administration and/or allow for lower dosing, resulting in improved patient compliance and/or lower costs in comparison to an antibody or antigen-binding fragment thereof with an unmodified Fc region.

In some embodiments, the Fc modification confers a longer circulation half-life. Typically, the modification relies on improving the interaction between the IgG Fc domain and the neonatal Fc receptor (FcRn), a ubiquitously expressed cellular receptor which binds to internalized IgG at endosomal pH (5.5-6.0), prevents lysosomal degradation and promotes recycling to the extracellular fluid (Roopenian and Akilesh, Nat. Rev. Immunol. 2007 September; 7(9):715-25). Fc engineering for higher FcRn binding affinity at endosomal pH has yielded several Fc mutations capable of improving IgG half-life, as assessed in non-human primates and in human FcRn transgenic mice models.

For example, the Fc modification may comprise an “LS” or so-called “XTEND™” mutation (M428L/N434S) developed by Xencor Corp. XTEND™ may provide an 11-fold increase in binding at pH 6.0 relative to wild-type IgG1, which is a 4.2-fold improvement in serum half-life in transgenic mice and 3.2-fold in non-human primates. As described in Zalevsky et al., 2010, Nat. Biotechnol., 2010 February; 28(2): 157-159, XTEND™ Fc was tested in xenograft mouse models that express human FcRn as either an anti-VEGF or anti-EGFR IgG1 antibody, which resulted in extended serum half-life as well as reduced tumor burden relative to those of wild-type IgG1. As described in Roth et al., 2018, XTEND™ has been adapted to ravulizumab (ALXN1210), resulting in a serum half-life of ˜49.7 days. Ravulizumab was approved by United States Food and Drug Administration on December 2018 for the treatment of paroxysmal nocturnal hemoglobinuria/hemolytic-uremic syndrome (Roth et al., Blood Adv., 2018 Sep. 11; 2(17):2176-2185). XTEND™ has also been adapted to VRC01-LS, which is under clinical evaluation for the prevention of human immunodeficiency virus (Gaudinski et al., PLoS Med. 2018 Jan. 24; 15(1):e1002493).

In some embodiments, an Fc portion of an antibody or Ab-CD4 conjugate described herein is modified to increase its affinity to Fc receptors. In some aspects, the Fc receptors are Fc receptors for IgG (FcγRs). For example, the Fc modification may comprise an G236A/S239D/A330L/1332E mutation within the Fc referred to as “GASDALIE” mutation as described in Ahmed at al., J Struct Biol 2016 April; 194(1):78-89 to enhance binding of Ab-CD conjugate to Fcγ receptors present on the effector cell surface.

sCD4 and CD4 Mimetics

CoRBS and Cluster A epitopes of the “closed” Env trimer residing on virions or infected cells become available for antibody recognition sequentially upon triggering with a CD4 moiety, such as sCD4 or CD4mc. As discussed above, sCD4 and CD4mc have been used to modulate Env conformation and expose CD4i epitopes, mostly within the CoRBS, in order to mediate direct neutralizing activity. Such non-conjugated molecules, however, exhibit a reduced ability to allow for antibody binding and subsequent viral neutralization and Fc-mediated effector functions.

Cluster of Differentiation 4, or “CD4,” is a glycoprotein located on the surface of immune cells and is the receptor to which HIV binds. CD4 contains four immunoglobulin domains d1, d2, d3, and d4, as well as a transmembrane domain and a cytoplasmic tail domain.

In certain embodiments, the CD4 moiety in the Ab-CD4 conjugates disclosed herein is selected from sCD4 compounds and CD4mc compounds. In certain embodiments, the sCD4 comprises or consists of the four immunoglobulin domains d1-d4. In certain other embodiments, the sCD4 comprises or consists of the d1d2 domain of sCD4, and in some embodiments, the sCD4 comprises or consists of the d1 domain of sCD4. sCD4 consisting of the d1d2 domain (CD4d1d2) is represented by the following sequence:

(SEQ ID NO: 69)
KKVVLGKKGDTVELTCTASQKKSIQFHWKNSNQIKILGNQGSFLTKGPSK
LNDRADSRRSLWDQGNFPLIIKNLKIEDSDTYICEVEDQKEEVQLLVFGL
TANSDTHLLQGQSLTLTLESPPGSSPSVQCRSPRGKNIQGGKTLSVSQLE
LQDSGTWTCTVLQNQKKVEFKIDIVVLAFQKASNT

Also within the scope of the present disclosure are synthetic peptide mimetics of CD4. These CD4mc compounds are small synthetic molecules (e.g., MW less than 600 Da) that bind HIV gp120 within the well-conserved Phe 43 cavity, near the binding site for CD4. The binding of CD4mc induces conformational changes in Env similar to those observed for CD4.

Several CD4mc compounds are known in the art, and they may be prepared according to any means recognized in the art. The CDmc moiety in the Ab-CD4 conjugates disclosed herein may be selected from any known CDmc in the art including small compounds and peptide-based compounds, including, for example, NBD-556, NBD-557, M48U1, JP-III-48, DMJ-I-228, MCG-IV-120, and BNM-III-170. Various CD4mc are disclosed, for instance, in Richard, J. et al., CD4 mimetics sensitize HIV-1-infected cells to ADCC, Proc. Natl. Acad. Sci. USA 2015; 112:E2687-2694; Madani, N. et al., A CD4-mimetic compound enhances vaccine efficacy against stringent immunodeficiency virus challenge, Nat. Commun. 2018; 9:2362; Melillo, B. et al., Small-Molecule CD4-Mimics: Structure-Based Optimization of HIV-1 Entry Inhibition, ACS Med. Chem. Lett. 2016; 7(3):330-334; and Van Herrewege, Y et al., CD4 mimetic miniproteins: potent anti-HIV compounds with promising activity as microbicides 2008; 61(4):818-26. In one embodiment, the CDmc is M48U1, and in one embodiment, the CDmc is BNM-III-170. BNM-III-170 has the following structure:

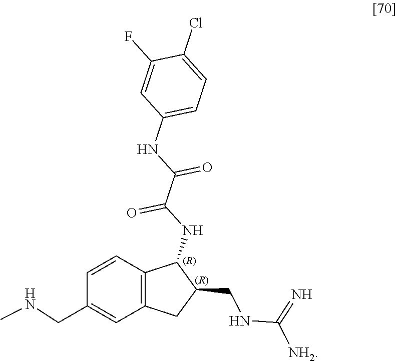

M48U1 is a small molecule with the following polypeptide sequence: Tpa-NLHFCQLRCKSLGLLGRCApTU1CACV-NH₂ (SEQ ID NO: 70), wherein Tpa is thiopropionyl, p is D-proline, and U₁ is Phe(p-cyclohexylmethoxy) [71].

In certain embodiments, at least 1, at least 2, at least 3, at least 4, at least 5, at least 6, at least 7, at least 8, at least 9, at least 10, at least 15, at least 20, at least 30, or at least 40 sCD4 molecules are conjugated to an antibody in an Ab-CD4 conjugate.

In certain embodiments, at least 1, at least 2, at least 3, at least 4, at least 5, at least 6, at least 7, at least 8, at least 9, at least 10, at least 15, at least 20, at least 30, or at least 40 CD4mc molecules are conjugated to an antibody in an Ab-CD4mc conjugate.

### Linkers

The linkers disclosed herein may be any suitable linker for conjugating the non-neutralizing or neutralizing antibody to the CD4 or CD4mc moiety such that the linker is of sufficient length and flexibility to allow for antigen binding, e.g., to allow for the potential interaction of all binding moieties to cognate targets on the Env trimer. In certain embodiments, the Ab-CD4 conjugates disclosed herein comprise two linkers, which may be the same or different, that link a single non-neutralizing or neutralizing antibody to two CD4 moieties.

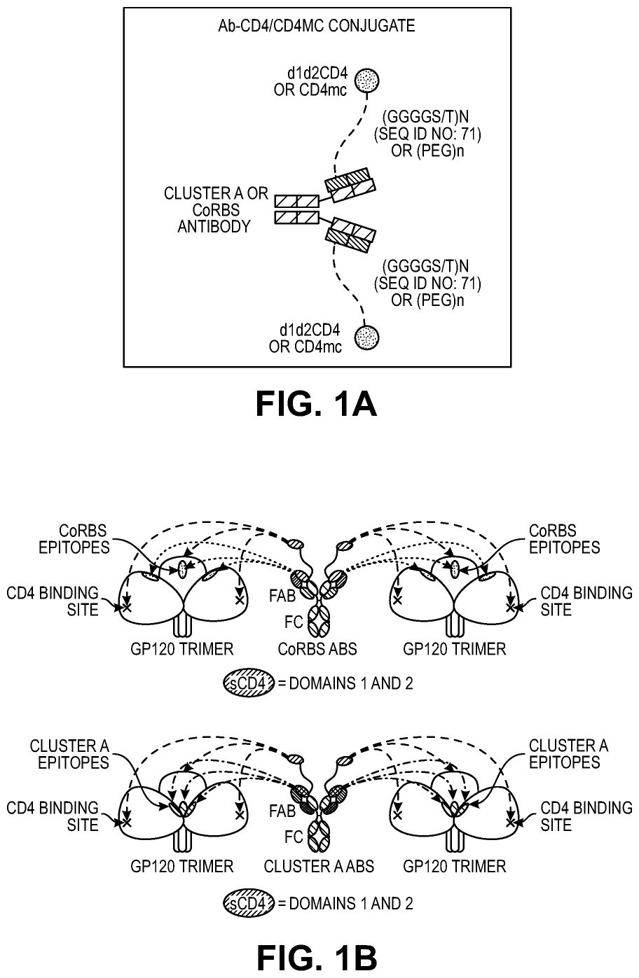

The linker may be conjugated to the non-neutralizing or neutralizing antibody at any desired site within the antibody frame. In certain embodiments, the linker is conjugated to the heavy chain constant region (e.g., C_{H}2), and in certain embodiments, the linker is conjugated to the light chain constant region (e.g., C_{L}). In certain embodiments, the Ab-CD4 conjugate disclosed herein comprises one non-neutralizing or neutralizing antibody having two linkers, each linking a CD4 moiety, such that the Ab-CD4 conjugate contains a total of four binding moieties, including the two CD4 binding moieties and the two Cluster A or CoRBS binding moieties on the non-neutralizing or neutralizing antibody located in the VH region, as depicted schematically, for example, in FIGS. 1A and 1B. In certain embodiments, the linker is a flexible linker selected from peptide linkers and polyethylene glycol linkers.

The length of the linker may be selected based upon structural information of the non-neutralizing or neutralizing antibody's epitopes, such as epitopes in the gp120 promoter region, in relation to the position of the CD4 binding site in the Env trimer. The linker length is compatible with the binding of the Fab arm and the CD4 moiety to the same gp120 promoter in the trimer, as well as, in certain embodiments, to optionally crosslink two adjacent gp120 promoters within the same trimer. For example, the linker length may vary depending on whether the Ab-CD4 conjugate contains a Cluster A antibody or a CoRBS antibody, as the Cluster A epitope is located further away from the CD4 binding site on the Env trimer than the CoRBS binding site. Accordingly, the linker of a Cluster A-CD4 conjugate may, in certain embodiments, be longer than the linker of a CoRBS-CD4 conjugate.

In certain embodiments of the disclosure, the flexible linker may be a peptide linker comprising from about 10-80 amino acids, such as about 20-70 amino acids, about 30-50 amino acids, about 35-45 amino acids, or about 40 amino acids. In certain embodiments, the linker may range from about 50 Å to about 200 Å in length, such as from about 50 Å to about 175 Å, from about 75 Å to about 155 Å, about 50 Å to about 60 Å, or about 150 Å to about 155 Å. In certain embodiments, the linker comprises glycine and serine or threonine residues, and in certain embodiments, the linker comprises a repeating sequence of GGGGS/T, i.e., (Gly-Gly-Gly-Gly-Xaa)n, wherein Xaa is serine or threonine and n is 2-16 (SEQ ID NO: 71). In certain embodiments, the linker has a sequence chosen from (G₄S)₆-(G₄T)₂ (SEQ ID NO: 72) and (G₄S)₈ (SEQ ID NO: 73), and in certain embodiments, the linker has a length of about 150 Å to about 155 Å, such as about 152 Å.

In certain embodiments, the flexible linker comprises polyethylene glycol (PEG). PEG linkers are well-known in the art and as disclosed herein may comprise, for example, about 4 to 50 PEG units, such as about 12 to 24 PEG units or 12 to 48 PEG units and/or may be about 40 Å to about 180 Å in length, such as about 45 Å to about 55 Å, or about 50 Å in length.

### Methods of Making Ab-CD4 Conjugates

The Ab-CD4 conjugates disclosed herein may be prepared by various methods known in the art. As would be recognized in the art, the methods employed will vary based on the CD4 moiety selected, as well as the desired linker, linker length, and conjugate site position on the antibody. In certain embodiments, the Ab-CD4 conjugate may be expressed from a single nucleic acid molecule in a vector, and in certain embodiments, the Ab-CD4 conjugate may be prepared by chemically conjugating a CD4 moiety to the antibody.

In certain embodiments wherein the CD4 moiety is sCD4, such as a d1d2 sCD4, and the linker is a peptide linker, the Ab-CD4 conjugates may be a single chimeric protein molecule expressed from a nucleic acid inserted into a vector. Any suitable host cell/vector system may be used for expression of the nucleic acid sequences encoding the antibodies or the Ab-CD4 conjugates disclosed herein. Bacterial, for example E. coli, and other microbial systems may be used, in part, for expression of CD4 compounds. Eukaryotic, e.g., mammalian, host cell expression systems may be used for production of larger antibody molecules, including complete antibody molecules or entire Ab-CD4 hybrid molecule. Suitable mammalian host cells include, for example, CHO, HEK293T, PER.C6, myeloma, and hybridoma cells. In certain embodiments, the Ab-CD4 conjugates disclosed herein may be produced in mammalian cells, such as mammalian Expi293F cells.

In certain embodiments, the antibody is linked to a d1d2CD4 via a (GGGGS/T)ₙ (SEQ ID NO: 71) linker, such that the construct is Ab-(GGGGS/T)ₙ-d1d2CD4, wherein n is 2-16. Such conjugates may be generated by transient transfection of heavy and light chain plasmids into an expression system, such as Expi293F cells. In certain embodiments, the construct may be a Ab-(GGGGS/T)ₙ-d1CD4 conjugate wherein n is 2-16, and in certain embodiments, the construct mat be a Ab-(GGGGS/T)ₙ-CD4 conjugate wherein CD4 comprises d1, d2, d3, and d4 and n is 2-16. In certain embodiments, the Ab-CD4 conjugates disclosed herein may be made using expression cells transfected with a plasmid encoding the desired CD4 domain linked to the N-terminus of the heavy chain of the antibody. Next, plasmids containing the appropriate kappa or lambda light chain of the antibody may be transfected with the Ab(C_{H})-CD4 construct to yield an Ab-CD4 conjugate comprising an intact antibody.

Following expression, the Ab-CD4 conjugates can be isolated or purified using any suitable technique known in the art. For example, methods that can be used include anion or cation exchange chromatography, phosphocellulose chromatography, hydrophobic interaction chromatography, affinity chromatography, immune-affinity chromatography, hydroxyapatite chromatography, lectin chromatography, molecular sieve chromatography, isoelectric focusing, gel electrophoresis, or any other suitable method or combination of methods. In certain embodiments, the expressed Ab-CD4 conjugates may be purified by affinity chromatography, such as Protein A affinity chromatography, and/or size exclusion chromatography.

In certain embodiments wherein the CD4 moiety is a CD4mc and the linker is a PEG linker, the CD4 moiety may be attached to the antibody moiety through a tRNA suppressor system, which system allows for site-specific incorporation of the linker through unnatural amino acids (UAAs) conjugated to the CD4 moiety via Click Chemistry. The tRNA suppressor system may be, for example, that described in Liu, W. et al., Genetic incorporation of multiple unnatural amino acids into proteins in mammalian cells, Nat. Methods 2007; 4:239-244. In such embodiments, the UAA, such as p-acetylphenylalanine, may first be incorporated at the desired conjugation site of the non-neutralizing or neutralizing antibody using the tRNA suppressor system as described (Liu, 2007); thereafter, the incorporated UAA can be conjugated to the CD4mc using Click Chemistry. In certain embodiments, a reassigned nonsense or frameshift codon may be used to encode the UAA, and an orthogonal aminoacryl-tRNA synthetase/tRNA pair specific to the UAA is used to deliver the UAA to co-translationally into the target antibody. After the aminoacryl-tRNA synthetase/tRNA pair site-specifically incorporates the UAA (e.g., p-acetylphenylalanine) into the antibody such that a UAA-Ab is expressed in an expression cell line, the keto group of the UAA may be selectively coupled to an alkoxy-amine derivatized compound of interest, such as PEG, via a stable oxime bond. In this way, PEG linkers may be used to attach the CD4 moiety to the non-neutralizing or neutralizing antibody moiety.

In certain embodiments, the conjugation/UAA incorporation site may be chosen individually for the particular antibody moiety to be conjugated, for example, within the regions not involved in forming an antigen binding site, such as within the unstructured loop regions of the Fab heavy or light chain constant domain.

In various other embodiments wherein the CD4 moiety is a CD4 me and the linker is a PEG linker, the Ab-CD4 conjugated may be prepared using a conjugation platform that uses site directed mutagenesis to incorporate cysteines into the antibody. Next, the disulfide bonds may be reduced and then reoxidized, followed by a classical conjugation reaction with maleimide or bromoacetamido moieties bearing a long and flexible PEG linker. Variable conjugation sites on the antibody may be selected, for example, based on available crystal structures of Fab-gp120 antigen complexes. For example, for the 17b, N12-i2, and C11 antibodies, conjugates sites at S74, Q61, and S56 of the heavy chains may be used, and for N5-i5, N60-i3, N12-i3, and A32 antibodies, conjugate sites at E125, S76, Q61, and S56 of the light chain may be used for cysteine conjugation. Finally, Click Chemistry is employed to conjugate the CD4mc moiety to the maleimide or bromoacetamido moieties comprising the PEG linker.

In the methods disclosed herein, the Fc N-glycan core structure of the antibody remains intact so as not to decrease Fc receptor binding. Variable conjugation sites and linker lengths are encompassed within the scope of the present disclosure.

### Vectors and Host Cells

In another aspect, the present disclosure is directed to a vector comprising an isolated polynucleotide comprising a nucleic acid molecule encoding any of the Ab-CD4 conjugates disclosed herein wherein the at least one linker is a peptide linker, or a complementary sequence of the present isolated polynucleotides. In some embodiments, the vector is a plasmid or cosmid. In other embodiments, the vector is a viral vector, wherein additional DNA segments can be ligated into the viral vector. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors”, can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

In some embodiments, the present disclosure is directed to a vector comprising a nucleic acid molecule that encodes a Ab-CD4 conjugates disclosed herein wherein the at least one linker is a peptide linker.

Generally, any system or vector suitable to maintain, propagate or express a polypeptide in a host may be used for expression of the Ab-CD4 conjugates disclosed herein. The appropriate DNA/polynucleotide sequence may be inserted into the expression system by any of a variety of well-known and routine techniques, such as, for example, those set forth in Sambrook et al., eds., Molecular Cloning: A Laboratory Manual (3rd Ed.), Vols. 1-3, Cold Spring Harbor Laboratory (2001).

In another aspect, the present disclosure is directed to a host cell comprising any of the vectors disclosed herein including the expression vectors comprising the polynucleotide sequences encoding the Ab-CD4 conjugates of the present disclosure. A wide variety of host cells are useful in expressing the present conjugates. Non-limiting examples of suitable host cells include well known eukaryotic and prokaryotic hosts, such as strains of E. coli, Pseudomonas, Bacillus, Streptomyces, fungi such as yeasts, and animal cells, such as CHO, Rl.l, B-W and L-M cells, African Green Monkey kidney cells (e.g., COS 1, COS 7, BSCl, BSC40, and BMT10), insect cells (e.g., Sf9), and human cells such as Expi293F cells and HOS cells.

Efficient expression of the present Ab-CD4 conjugates depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the Ab-CD4 conjugates of the present disclosure, one skilled in the art will be able to select the proper vectors, expression control sequences, and hosts without undue experimentation to accomplish the desired expression without departing from the scope of this disclosure.

### Pharmaceutical Compositions and Kits

Further disclosed herein are pharmaceutical compositions comprising the Ab-CD4 conjugates of the present disclosure. The pharmaceutical compositions disclosed herein can take any suitable form, including the form of solutions, suspensions, emulsion, tablets, pills, pellets, capsules, capsules containing liquids, powders, and sustained-release formulations, injectants, and combinations thereof.

In certain embodiments disclosed herein, the pharmaceutical composition may comprise a single Ab-CD4 conjugate of the present disclosure, such as a Cluster A-CD4 conjugate. Further disclosed herein is a pharmaceutical composition comprising a mixture of at least two different Ab-CD4 conjugates, such as a mixture of Cluster A-CD4 conjugates and CoRBS-CD4 conjugates. Further disclosed herein is a pharmaceutical composition comprising a mixture of one Ab-CD4 conjugate and unconjugated antibody, such as a mixture of Cluster A-CD4 conjugates and unconjugated CoRBS antibody or a mixture of CoRBS-CD4 conjugate and unconjugated Cluster A antibody. The pharmaceutical compositions disclosed herein comprising Ab-CD4 conjugates can be administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. The pharmaceutical compositions may be administered parenterally, when possible, at the target cell site, or intravenously. Intravenous or subcutaneous administration of the Ab-CD4 conjugates is preferred in certain embodiments. The pharmaceutical compositions disclosed herein are administered to a patient or subject systemically, parenterally, or locally.

For parenteral administration, the Ab-CD4 conjugates can be formulated in a unit dosage injectable form (e.g., solution, suspension, or emulsion) in association with a pharmaceutically acceptable carrier.

Suitable pharmaceutically acceptable carriers include, but are not limited to, nontoxic buffers such as phosphate, citrate, and other organic acids; salts such as sodium chloride; antioxidants including ascorbic acid and methionine; preservatives (e.g., octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG). The compositions disclosed herein may be chosen from any suitable form, including injectable suspensions, solutions, sprays, lyophilized powders, syrups, and elixirs. Additional examples of carriers include water, saline, Ringer's solution, dextrose solution, and 5% human serum albumin. Nonaqueous carriers such as fixed oils and ethyl oleate and liposomes may also be used. The carriers disclosed herein may contain minor amounts of additives such as substances that enhance isotonicity and chemical stability, e.g., buffers and preservatives. The Ab-CD4 conjugates may be formulated in such carriers at concentrations of, for example, about 1 mg/ml to 10 mg/ml.

The dose and dosage regimen may depend upon a variety of factors, such as the nature of the infection and the characteristics of the particular Ab-CD4 conjugates to be administered, e.g., its therapeutic index, the patient, and the patient's history. Generally, a therapeutically effective amount of at least one Ab-CD4 conjugate is administered to a patient. In particular embodiments, the amount of Ab-CD4 conjugate administered is in the range of about 0.1 mg/kg to about 20 mg/kg of patient body weight. Depending on the type and severity of the infection, about 0.1 mg/kg to about 20 mg/kg body weight (e.g., about 0.1-15 mg/kg/dose) of antibody is an initial candidate dosage for administration to the patient, whether, for example, by one or more separate administrations, or by continuous infusion. The progress of the therapy may be readily monitored by conventional methods and assays and based on criteria known to the physician or other persons of skill in the art.

In yet another embodiment, there is provided a kit for performing diagnostic and prognostic assays using the Ab-CD4 conjugates disclosed herein. Kits may include a suitable container comprising an Ab-CD4 conjugate in either labeled or unlabeled form. In addition, when the Ab-CD4 conjugate is supplied in a labeled form suitable for an indirect binding assay, the kit further includes reagents for performing the appropriate indirect assay. For example, the kit includes one or more suitable containers including enzyme substrates or derivatizing agents, depending on the nature of the label. Control samples and/or instructions may also be included.

### Methods

Disclosed herein are methods of killing HIV-infected cells through an Fc-mediated effector function, as well as methods of using pharmaceutical compositions comprising the Ab-CD4 or Ab-CD4mc conjugates to treat or prevent HIV infection and methods of reducing or eliminating the latent HIV reservoir. Methods disclosed herein further include methods for preventing an increase in HIV virus titer, virus replication, virus proliferation or an amount of an HIV viral protein in a subject, said methods comprising administering an effective amount of the Ab-CD4 or Ab-CD4mc conjugates disclosed herein to the subject.

In certain embodiments, there are disclosed herein methods of killing HIV-infected cells through an Fc-mediated effector function comprising contacting the HIV-infected cell with an Ab-CD4 or Ab-CD4mc conjugate as disclosed herein, thereby neutralizing HIV and eliminating HIV-infected cells. The Ab-CD4 conjugates disclosed herein comprising either intact CoRBS or Cluster A antibodies exhibit effective virus recognition and ADCC or ADCP killing of HIV-infected cells, as demonstrated in the Examples below. The level of binding and ADCC and ADCP activities of the Ab-CD4 or Ab-CD4mc conjugates disclosed herein having Cluster A specificity were comparable to those observed for the Ab-CD4 or Ab-CD4mc conjugates having CoRBS specificity, indicating similar epitope exposure. This indicates that Cluster A Ab-CD4 conjugates acting through multivalent binding are capable of overcoming the energy barrier required to expose the Cluster A targets residing at the infected cell surface.

The Ab-CD4 or Ab-CD4mc conjugates disclosed herein, including both those having Cluster A or CoRBS specificity, further show efficient, cross-clade neutralization of Tier 1 and 2 viral strains. Tiers are categorized based on the frequency by which the Env trimer exists in a closed, open, or intermediate conformation, wherein Tier 1 viruses are more frequently open or intermediate, and Tier 2 viruses are more frequently closed. The Cluster A region of the Env trimer is known to be targeted by antibodies lacking any neutralizing activity, including for easy-to-neutralize Tier 1 viruses. The Cluster A-CD4 conjugates disclosed herein, however, are capable of stabilizing Env in more “open” conformations, thereby resulting in novel non-neutralizing Cluster A antibody conjugates capable of potent ADCC and ADCP and acquiring neutralizing activity against HIV.

In certain embodiments of all aspects of the disclosure, the Ab-CD4 or Ab-CD4mc conjugates disclosed herein are used in a method of detecting binding of Ab-CD4 or Ab-CD4mc antibodies to antigens, such as Env. Such methods may include, but are not limited to, antigen-binding assays that are well-known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry. In certain embodiments disclosed herein, the binding capacity of the Ab-CD4 conjugates to unliganded cell-surface Env may be evaluated by cell-based ELISA.

In certain embodiments, there are further disclosed herein methods of treating or preventing HIV infection comprising administering to a subject pharmaceutical composition comprising an Ab-CD4 or Ab-CD4mc conjugate as disclosed herein. In certain embodiments, the method may include co-administering both an anti-Cluster A-CD4 conjugate and an anti-CoRBS-CD4 conjugate. Co-administering, as used herein, may be in a sequential manner, as well as administration of these agents in a substantially simultaneous manner, such as in a single mixture/composition or in doses given separately, but nonetheless administered substantially simultaneously to the subject, for example at different times in the same day or 24-hour period. Such co-administration of Cluster A-CD4 conjugates and CoRBS-CD4 conjugates can be provided as a continuous treatment lasting up to hours, days, weeks, or months. In some embodiments, the subject is human, including a human with HIV infection or at risk for HIV-related diseases or disorders.

Subjects at risk for HIV-related diseases or disorders include patients who have come into contact with an infected person or who have been exposed to HIV in some way. Administration of a prophylactic agent can occur prior to the manifestation of symptoms characteristic of HIV-related disease or disorder, such that a disease or disorder is prevented or delayed in its progression.

In certain aspects, the methods of treating or preventing HIV infection may further comprise co-administering other agents suitable for the treatment of HIV infection, such as anti-retroviral therapies. Anti-retroviral therapies that may be co-administered with the Ab-CD4 conjugates disclosed herein may include, for example, nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors (such as bevirimat and vivecon), or broad spectrum inhibitors, such as natural antivirals.

Further disclosed herein is a method of reducing or eliminating the latent HIV reservoir comprising administering the Ab-CD4 conjugates disclosed herein to a subject infected with HIV. In certain embodiments disclosed herein, the impact of HIV Nef- and Vpu-mediated CD4 down-regulation is diminished, as such down-regulation leads to a reduction in CD4-inducible ADCC and ADCP targets on infected cells. Because the Ab-CD4 conjugates disclosed herein use the potential of the non-neutralizing antibodies to kill the HIV infected cells through Fc receptor-mediated mechanisms, the capacity of non-neutralizing antibodies to recognize infected cells is reduced by HIV accessory viral protein U (Vpu) and negative regulatory factor (Nef) protein, two well-established regulators of cell-surface CD4 expression. By decreasing the levels of CD4 on the surface of the infected/budding target cell, Vpu and Nef disrupt the exposure of epitopes recognized by the CD4i antibodies and assist in creating a latent reservoir of HIV-infected cells.

One strategy for diminishing or eradicating the latent reservoir is the “shock-and-kill” strategy, which seeks first to bring latent cells back into a state of viral production and replication and then seeks to kill those infected cells. Although shock-and-kill strategies represent promising approaches to HIV eradication, latently infected cells in which viral production has been induced by latency-reversing agents are unlikely to be depleted in the absence of an efficient immune response. Several barriers are known to prevent the efficient killing of these cells by autologous CD8+ T cells, including, for example the paucity of HIV specific CD8 T cells in fully suppressed individuals on ART; the downregulation of MHC at the surface of infected cells that prevents antigen presentation; and the accumulation of CTL immune escape variants in the latent reservoir.

Accordingly, the methods disclosed herein of diminishing or eradicating the latent reservoir of infected cells after viral reactivation relies on immune cells to mediate ADCC and/or ADCP. Through ADCC, effector cells such as NK cells and monocytes can kill infected cells expressing Env through recognition by HIV-specific antibodies. Because the HIV Vpu and Nef proteins keeps Env-CD4 complexes, the main target for ADCC, off of the cell surface, this immune mechanism is inefficient. However, as disclosed herein, the Ab-CD4 conjugates are able to push the HIV Env protein into the CD4-bound conformation, resulting in sensitization of HIV infected cells to ADCC. Accordingly, the Ab-CD4 conjugates disclosed herein may be used to kill HIV-infected latent reservoir cells after the viral production of such latent reservoir cells has been induced by at least one latency reversing agent.

In the methods disclosed herein, any latency reversing agent known in the art may be used. Latency reversing agents are disclosed, for example, in Ait-Ammar, A. et al., Current Status of Latency Reversing Agents Facing the Heterogeneity of HIV-1 Cellular and Tissue Reservoirs, Front. Microbiol. 2020; 10:3060. In certain embodiments, the latency reversing agent may be selected from the group consisting of PKC agonists (e.g., ingenols), MAPK agonists (e.g., procyanidin trimer C1), CCR5 antagonists (e.g., maraviroc), Tat vaccines (e.g., Tat Oyi vaccine, Tat-R5M5 protein), SMAC mimetics (e.g., SBI-0637142, birinapant), inducers of P-TEFb release (e.g., JQT, I-BET, I-BET151, OTX015, UMB-136, MMQO, CPI-203, RVX-208, PFI-1, BI-2536, BI-6727, HMBA), activators of Akt pathway (e.g., disulfiram), benzotriazole derivatives (1-hydroxybenzotriazol), epigenetic modifiers such as HDACis (e.g., TSA, trapoxin, SAHA, romidepsin, Panobinostat, entinostat, givinostat, valproic acid, MRK-1/11, AR-42, fimepinostat, chidamide), HMTis (e.g., chaetocin, EPZ-6438, GSK-343, DZNEP, BIX-01294, UNC-0638), and DNMTis (e.g., 5-AzaC, 5-AzadC), and immunomodulatory latency reversing agents such as TLR agonists (e.g., TLR2/Pam3CSK4), TLR7/GS-9620, TLR8, TLR9/MGN 1703), IL-15 agonists (e.g. ALT-803), and immune checkpoint inhibitors (e.g., anti-PD-1, niolumab, pemprolizumab, anti-CTLA-4, ipilimumab). For example, in certain embodiments the latency reversing agent may be a PKC agonist, such as ingenol.

By diminishing or eradicating the latent reservoir of HIV-infected cells, the Ab-CD4 conjugates disclosed herein may be useful in a variety of applications including, for example, therapeutic treatment methods, such as the treatment, cure, functional cure, or prevention of HIV infection. All of the methods of use disclosed herein may be in vitro, ex vivo, or in vivo methods.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

Unless indicated otherwise in these Examples, the methods involving commercial kits were done following the instructions of the manufacturers.

### Statistical Analysis:

Statistics were analyzed using GraphPad Prism version 8.4.3 (GraphPad, San Diego, CA, USA). Each data set was tested for statistical normality, and this information was used to apply the appropriate (parametric or nonparametric) statistical test. P values of <0.05 were considered significant, and significance values are indicated as follows: *, P<0.05; **, P<0.01; ***, P<0.001; ****, P<0.0001.

### Cell Lines and Isolation of Primary Cells.

HEK293T human embryonic kidney cells, HeLa TZMbl cells and human osteosarcoma (HOS) cells were grown as previously described [23, 48]. Expi293F™ cells (Thermo Fisher Scientific) were cultured in Gibco™ Expi293™ Expression Medium supplemented with 100 IU penicillin and 100 μg/ml streptomycin solution at 37° C., 8% CO₂, 90% humidity and shaking at 125 rotations per minute, according to the manufacturer's protocol. Primary human peripheral blood mononuclear cells (PBMCs) and CD4+ T cells were isolated, activated, and cultured as previously described [41]. PBMCs were obtained by leukapheresis and CD4+ T lymphocytes were purified from resting PBMCs by negative selection using immunomagnetic beads per the instructions of the manufacturer (StemCell Technologies, Vancouver, BC, Canada) and were activated with phytohemagglutinin-L (10 μg/ml) for 48 h and then maintained in RPMI 1640 complete medium supplemented with recombinant interleukin-2 (rIL-2) (100 U/ml).

### Plasmids, Vectors and Proviral Constructs.

The JR-CSF IMC and the pNL4.3 Nef-Luc Env-plasmid were obtained from the AIDS Research and Reference Reagent Program, Division of AIDS, NIAID, NIH. The JRFL and pHIV-1_{AD8} IMCs were previously described [63, 64]. Plasmid expressing the full-length Envs HIV-1_{JRFL}, HIV-1_{YU2}, HIV-1_{CM244}, HIV-1_{C1086}, HIV-1_{BG505} and HIV-1_{ZM109} were previously reported [23, 65-67]. The CD4-IgH plasmid contains domains 1 and 2 of CD4 fused to the heavy chain through a flexible 40 amino acids linker (6 repeats of G₄S and 2 repeats of G₄T motifs). Two restriction sites NheI at the 5′-end and BamHI at the 3′-end were incorporated into the CD4-polypeptide linker plasmid which was then inserted into pACP-tag(m)-2 plasmid (New England Biolabs) to obtain pACP-CD4. The IgGH gene lacking the leader sequence was amplified by PCR with the given primers and purified using the MinElute Reaction cleanup kit (Qiagen), followed by digestion with BamHI-Hf and NotI restriction enzymes (New England Biolabs). The digested fragment was purified by agarose gel electrophoresis, then ligated into BamHI and NotI sites of pACP-CD4 to afford the respective pACP-CD4-IgH vector which was transformed into NEB® 5-alpha F′Iq Competent E. coli according to the manufacturer's protocol (New England Biolabs). Vectors containing CD4 and heavy chain genes were then sequenced and compared with the original heavy chain sequences. In preparation for larger scale protein production, plasmids were grown under ampicillin selection and purified using GeneJET plasmid Midiprep Kit (Thermo Scientific), following the protocol specified by the manufacturer. The vesicular stomatitis virus G (VSV-G)-encoding plasmid pSVCMV-IN-VSV-G was previously reported [68].

### Generation of Stable CD4-IgH Cell Lines.

Expi293 cells were seeded at 1×10⁶ cells/ml (viability >90%) and transfected with CD4-IgH plasmid encoding domains 1 and 2 of CD4 fused to the N-terminus of the heavy chain of the non-neutralizing antibody using EndoFectin™ Max transfection reagent (GeneCopoeia) following the manufacturer's protocol. Transfected cells were incubated for 5 days at 37° C., in a humidified atmosphere of 8% CO₂, at 125 rpm. Then, cell culture supernatants were replaced and maintained using Gibco™ Expi293™ Expression Medium supplemented with 50 μg/ml Geneticin until cell viability reached and remained >90%.

### Expression and Purification of CD4-IgG Conjugates.

CD4-conjugated antibodies were produced in stable cell lines via transient transfection of plasmid coding light chains using EndoFectin™ Max transfection reagent (GeneCopoeia). Transfected cells were incubated at 37° C., in a humidified atmosphere of 8% CO₂, on an orbital shaker (125 rpm). Five days post-transfection, cell culture supernatants were collected, centrifuged and filtered through 0.22 μm PES membrane to remove cell debris. Antibody conjugates were affinity purified by protein A affinity chromatography according to the manufacturer's instructions. The eluted proteins were concentrated, buffer-exchanged with DPBS buffer (pH 7) and purified over Superdex 200 Increase 10/300 GL column (GE Healthcare) in DPBS buffer. The purified proteins were pooled, concentrated with Amicon Ultra-15 centrifugal filters (MWCO 30,000, Millipore), analyzed by reducing SDS-PAGE with Coomassie Blue staining, and further purified by size exclusion chromatography (Superdex 200 Increase 5/150 GL column, GE Health Sciences). Finally, protein concentration was quantified by the Pierce BCA protein assay (Thermo Scientific), aliquoted and stored at −20° C.

### Negative Stain Electron Microscopy (NS-EM).

The SOSIP CD4i Abs complexes were prepared by mixing a 3:1 molar ratio of BG505 SOSIP.664 and CD4i Abs. The resulting mixture was incubated at room temperature for 2 hours and purified by SEC on a Superdex 200 Increase 10/300 GL column. Fraction containing SOSIP-CD4Abs complexes were analyzed by NS-EM. A 5 μL of sample were applied for 1 minute to carbon-coated 400 Cu mesh grid which had been glow discharged at 25 mA for 2 minutes, followed by negative staining with 2% Uranyl Acetate for 1 minute. Data was collected using a JEOL LEM-1011 microscope operating at 100 keV, with a magnification of 120,000×.

### Viral Production and Infection.

VSV-G-pseudotyped HIV-1 JRFL virus was produced and titrated as previously described [22]. Viruses were then used to infect activated primary CD4+ T cells from healthy HIV-1-negative donors by spin infection at 800×g for 1 h in 96-well plates at 25° C. For the viral neutralization assay, TZM-bl cells were infected with either single-round luciferase-expressing HIV-1 pseudovirions or fully replicative WT viruses. Briefly, 293T cells were transfected by the calcium phosphate method with the proviral vector pNL4.3Luc Env- and a plasmid expressing indicated HIV-1 Env at a ratio of 2:1 or with full IMCs constructs. Two days after transfection, the cell supernatants were harvested. Each virus preparation was frozen and stored in aliquots at −80° C. until use.

### Virus Capture Assay.

The assay was modified from a previously published method [35]. Pseudoviral particles were produced by transfecting 2×10⁶ HEK293T cells with pNL4.3 Nef-Luc Env- (3.5 μg), pSVCMV-IN-VSV-G (1 μg) and plasmid (2.5 μg) encoding for JRFL full length Env using the standard calcium phosphate protocol. 48 hours later, virion-containing supernatants were collected, and the cell debris was removed through centrifugation (486×g for 10 min). To immobilize antibodies on ELISA plates, white Pierce™ protein A coated 96-well plates (Thermo Fisher Scientific, Waltham, MA, USA.) were incubated with 5 μg/ml of the tested antibodies diluted in 100 μL phosphate-buffered saline (PBS) overnight at 4° C. Unbound antibodies were removed by washing the plates twice with PBS. Plates were subsequently blocked with 3% BSA in PBS for 1 hour at room temperature. After two washes with PBS, 200 μL of virion-containing supernatant was added to the wells in the presence or absence of sCD4 (10 μg/ml). Viral capture by any given antibodies was visualized by adding 1 ×10⁴ HIV-1-resistant HEK293T cells in full DMEM medium per well. Forty-eight hours post-infection, cells were lysed by the addition of 30 μL of passive lysis buffer (Promega, Madison, WI, USA.) and three freeze-thaw cycles. An LB941 TriStar luminometer (Berthold Technologies) was used to measure the luciferase activity of each well after the addition of 100 μL of luciferin buffer (15 mM MgSO₄, 15 mM KH₂PO₄ (pH 7.8), 1 mM ATP, and 1 mM dithiothreitol) and 50 μL of 1 mM D-luciferin potassium salt (Prolume, Randolph, VT, USA.). Luciferase signals were then normalized to those obtained with the 2G12 antibody.

### Viral Neutralization Assay.

TZM-bl target cells (NIH AIDS reagent program) were seeded at a density of 1×10⁴ cells/well in 96-well luminometer-compatible tissue culture plates (Perkin Elmer) 24 hours before infection. 100 μL of recombinant viruses were mixed and incubated with 100 μL of Ab (+/−sCD4) or Ab-CD4 conjugate for 1 h at 37° C. For the competition experiments, Ab-CD4 were pre-incubated with 10 μg/ml of recombinant gp120 proteins prior incubation with recombinant virus. Each mix of virions and Ab or Ab-CD4 conjugate was then split into two and added to the target cells followed by incubation for 4 hours at 37° C.; 100 μL of fresh DMEM 5% FCS 1% P en-Strep was then added to the cells, which had been incubated for an additional 48 hours at 37° C. Cells were then lysed by the addition of 30 μl of passive lysis buffer (Promega) followed by one freeze-thaw cycle. An LB941 TriStar luminometer (Berthold Technologies) was used to measure the luciferase activity of each well after the addition of 100 μl of luciferin buffer (15 mM MgSO₄, 15 mM KPO₄ [pH 7.8], 1 mM ATP, and 1 mM dithiothreitol) and 50 μl of 1 mM d-luciferin potassium salt (Prolume). The neutralization half-maximal inhibitory concentration (IC₅₀) has been calculated with GraphPad Prism version 8.0.1.

### Recombinant Proteins.

Soluble CD4 (sCD4) was produced and purified as previously described [23]. The recombinant gp120 proteins ΔV1V2V3V5 WT, ΔV1V2V3V5 D368R and ID2 were produced and purified as previously reported [43, 58].

### ELISA.

The capacity of recombinant gp120 proteins to interact with CD4, CoRBS and Cluster A Abs was tested by ELISA as previously described [69]. Bovine serum albumin (BSA) and the recombinant gp120 proteins (ΔV1V2V3V5 WT, ΔV1V2V3V5 D368R and ID2) were prepared in PBS (0.1 μg/mL) and adsorbed to MaxiSorp; Nunc plates (Thermo Fisher Scientific, Watham, MA, USA) overnight at 4° C. BSA was used as a negative control. Coated wells were subsequently blocked with blocking buffer (Tris-buffered saline (TBS) containing 0.1% Tween 20 and 2% [wt/vol] BSA) for 90 minutes at room temperature. Wells were then washed 4 times with washing buffer (Tris-buffered saline [TBS] containing 0.1% Tween 20). Antibodies (17b, N5i5 or CD4-g) were diluted in blocking buffer and incubated for 120 minutes at room temperature. Wells were then washed 4 times with washing buffer. This w % as followed by incubation of HRP conjugated antibody specific for the Fc region of human IgG (Pierce) for 90 minutes at room temperature. Wells were then washed 4 times with washing buffer. HRP enzyme activity was determined after the addition of a 1:1 mix of Western Lightning ECL reagents (Perkin Elmer Life Sciences, Waltham, MA, USA). Light emission was measured with an LB 941 TriStar luminometer (Berthold Technologies, Bad Wildbad, Germany).

### Cell-Based ELISA.

Recognition of trimeric HIV-1_{JRFL} envelope glycoprotein (Env) ΔCT at the surface of HOS cells was performed by cell-based ELISA, as previously described [49]. HOS cells were seeded in T-25 flasks (2×10⁶ cells per flask) and transfected the next day with a total of 12 μg of pcDNA3.1 expressing the codon-optimized HIV-1_{JRFL} EnvΔCT, either WT or containing the CD4-binding site D368R mutation, per flask using the standard polyethylenimine (PEI; Polysciences Inc., PA, USA) transfection method. Twenty-four hours after transfection, cells were plated in 384-wells plates (2×10⁴ cells per well). One day later, cells were incubated in blocking buffer (washing buffer [25 mM Tris (pH 7.5), 1.8 mM CaCl₂), 1.0 mM MgCl₂ and 140 mM NaCl] supplemented with 10 mg/ml non-fat dry milk and 5 mM Tris [pH 8.0] for 30 minutes and then co-incubated for 1 hour with indicated Ab-CD4 or unconjugated Ab (1 μg/ml) in the presence or absence of soluble CD4 (sCD4) (3 μg/ml) in phosphate-buffered saline [PBS] diluted in blocking buffer. Cells were then washed five times with blocking buffer and five times with washing buffer. A HRP conjugated antibody specific for the Fc region of human IgG (Pierce) was then incubated with all the samples for 45 minutes and then washed again as just described. All incubations were done at room temperature. To measure the HRP enzyme activity, 20 μl of a 1:1 mix of Western Lightning oxidizing and enhanced luminol reagents (Perkin Elmer Life Sciences) was added to each well. Chemiluminescence signal was acquired for 1 see/well with the LB 941 TriStar luminometer (Berthold Technologies).

### Flow Cytometry Analysis of Cell Surface Staining.

Cell surface staining of infected cells was performed as previously described [41]. Binding of cell surface HIV-1 Env by Ab-CD4 conjugates or unconjugated Ab (10 μg/ml) was performed at 48 hours post-infection in the presence or absence of sCD4 (10 μg/ml). For competition experiments, Ab or Ab-CD4 conjugates were pre-incubated for 30 minutes at room temperature with purified soluble gp120 ΔV1V2V3V5 D368R or ID2 protein (10 μg/ml) prior incubation with infected cells for 45 minutes. Cells were then washed twice with PBS and stained with 2 μg/ml goat anti-human (Alexa Fluor 647; Invitrogen) secondary Abs for 15 min in PBS. After two more PBS washing, cells were fixed in a 2% PBS-formaldehyde solution. To evaluate F240 epitope exposure, infected cells were stained with Alexa-Fluor 647-conjugated F240 Abs in the presence of Ab-CD4 or unconjugated Ab (10 μg/ml)+/−sCD4 (10 μg/ml). Infected cells were then stained intracellularly for HIV-1 p24, using a Cytofix/Cytoperm fixation/permeabilization kit (BD Biosciences, Mississauga, ON, Canada) and fluorescent anti-p24 MAb (phycoerythrin [PE]-conjugated anti-p24, clone KC57; Beckman Coulter/Immunotech). The percentage of infected cells (p24+) was determined by gating the living cell population on the basis of viability dye staining (Aquavivid; Thermo Fisher Scientific). Samples were acquired on an LSR II cytometer (BD Biosciences), and data analysis was performed using FlowJo vX.0.7 (Tree Star, Ashland, OR, USA).

### FACS-Based ADCC Assay.

Measurement of ADCC using the FACS-based assay was performed at 48 hours post-infection as previously described [48]. Infected primary CD4⁺ T cells were stained with Aquavivid viability dye and cell proliferation dye (eFluor670; eBioscience) and used as target cells. Autologous PBMC effector cells, stained with another cellular marker (cell proliferation dye eFluor450; eBioscience), were added at an effector/target ratio of 10:1 in 96-well V-bottom plates (Corning, Corning, NY). Antibodies (+/−sCD4) or Ab-CD4 conjugates were added to appropriate wells, and the cells were incubated for 15 minutes at room temperature. The plates were subsequently centrifuged for 1 minute at 300×g and incubated at 37° C. and 5% CO₂ for 5 hours before being fixed in a 2% PBS-formaldehyde solution. Samples were acquired on an LSR II cytometer (BD Biosciences), and data analysis was performed using FlowJo vX.0.7 (Tree Star). The percentage of ADCC resulting from gating performed on infected lived target cells was calculated with the following formula: (percentage of p24+ cells in targets plus effectors)−(percentage of p24+ cells in targets plus effectors plus Abs)/(percentage of p24+ cells in targets).

### Example 1—Design and Expression of Single Chain Ab-CD4 Conjugates

The single chain Ab-CD4 conjugates comprising a CoRBS or Cluster A specific nnAb linked to the C-terminus of sCD4 (domain 1 and 2, residues 26-208 of extracellular domain of human CD4) (SEQ ID NO: 69) via a flexible 40 amino acid-(-(Gly₄—Ser)₆-(Gly₄—Thr)₂) (SEQ ID NO: 72) linker were prepared as described above. The linker was attached to the N-terminus of the heavy chain (IgGH) of the mAb IgG1, resulting in molecule in which 2 sCD4 domains were attached to a single IgG1. For each epitope specificity, 2 antibodies were tested as the IgG1 arm of the Ab-CD4 conjugate molecule. A32, the prototype antibody of the Cluster A region, and N5-i5, an antibody isolated from an HIV-1 infected individual capable of potent ADCC against CD4i Env targets, were selected to represent the Cluster A region. A32 and N5-i5 recognize largely overlapping epitopes that map to the highly conserved C1-C2 portion of the Cluster A region of CD4-triggered gp120. For the CoRBS-specific antibodies, 17b, an antibody recognizing a conserved epitope within the bridging sheet of the CoRBS, and X5, an antibody combining the elements of the highly conserved bridging sheet of the CoRBS with elements of the V3 loop stem, were selected. The length of the (G₄S/T)ₙ-linker (SEQ ID NO: 71) was selected based upon structural information of the antibody epitope within the gp120 promoter in relation to the position of the CD4 binding site in the Env trimer. The selected linker length is compatible with the binding of the F_{ab} arm and sCD4 to the same gp120 promoter in the trimer as well as possibly to crosslink two adjacent gp120 protomers within the same trimer. The selected linker length was similar to what was used to develop conjugates of a single chain variable fragment (scFv) of 17b and sCD4 that showed HIV-1 neutralizing activity [38]. ScFv conjugates with long linkers (35-40 amino acid residues) displayed stronger neutralizing activity as compared to the same constructs with a shorter linker (5-20 amino acids residues). Therefore, the Ab-CD4 conjugates herein were designed with a flexible 40-amino acid linker (six repeats of the G₄S motif and two repeats of the G₄T motif).

Ab-CD4 conjugates were produced in mammalian Expi293F cells as discussed above. First, an Expi293F cell line was developed to stably express domains 1 and 2 of CD4 fused to the N-terminus of the heavy chain of the selected nnAb. The plasmid containing the appropriate kappa or lambda light chain was then transfected. The production of Ab-CD4 conjugates using this method typically yielded 6-19 mg of properly folded product per liter of culture. The purification protocol included Protein A affinity chromatography, followed by size exclusion chromatography (SEC). The calculated molecular weight of the Ab-CD4 conjugate is about 200 kDa, as compared to about 150 kDa for wild-type mAb. The SEC polishing step was performed to remove protein aggregates and/or dimers. Under the reducing condition of SDS-PAGE, all the mAbs, alone or linked to sCD4, were resolved at the expected molecular weights. The heavy chains of all Ab-CD4 conjugates migrated to form a band with a molecular weight of 75 kDa that corresponds to the sum of the heavy chain (-50 kDa) plus the d1d2 domain of CD4 (-25 kDa). In SEC analyses, one distinct peak corresponding to the Ab-CD4 conjugates was observed eluting earlier than a 158 kDa peak of mAb alone.

### Example 2—Single Chain Ab-CD4 Conjugates Recognize the Unliganded Env Present at the Surface of HIV-1 Infected Cells

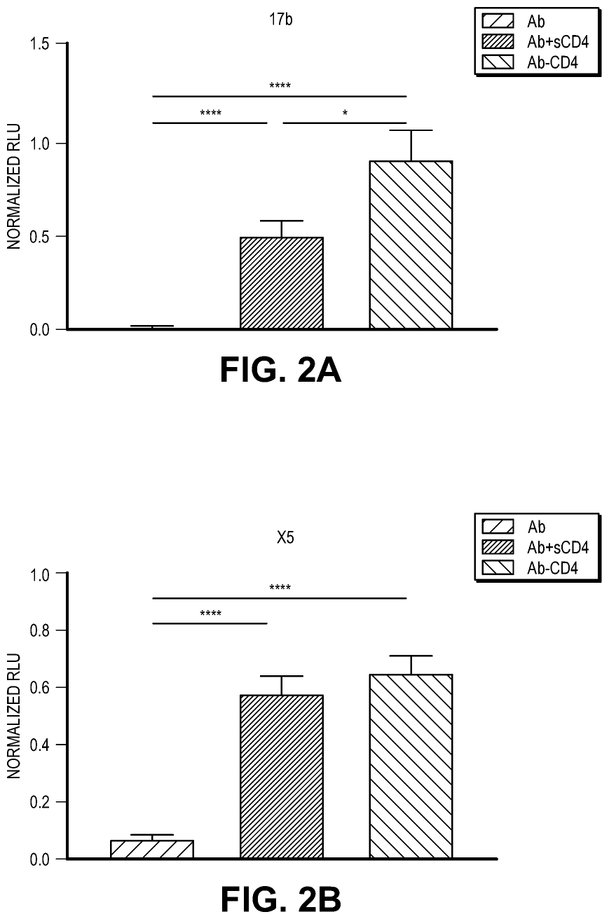

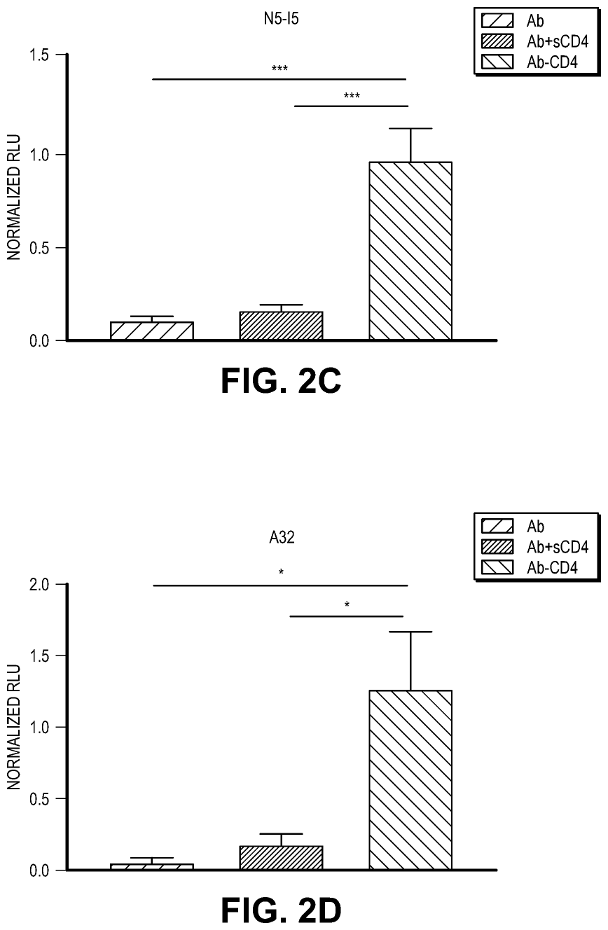

The Ab-CD4 conjugates prepared as discussed above were developed to recognize the “closed” Env trimer available at the surface of HIV-1 infected cells and to trigger and expose CD4i epitopes. Therefore, the binding capacity of the Ab-CD4 conjugates to unliganded cell-surface Env was evaluated using a previously described cell-based ELISA [48, 49]. Briefly, HOS cells were transfected with a plasmid encoding the HIV-1_{JRFL} Env. Two days later, transfected cells were incubated with (1) antibodies alone (17b, X5, A32, and N5-i5); (2) a mixture of antibodies plus sCD4 (3 μg/ml); or (3) Ab-CD4 conjugates. Binding was detected using HRP-conjugated secondary antibodies and is reported as normalized relative luminescent units (RLU), with ±SEM from at least 4 independent experiments performed in quadruplicate, with the signal obtained from cells transfected with an empty pcDNA3.1 plasmid (no Env) subtracted, normalized to Env levels as determined by bNAb 2G12. The results are shown below in Table 1 and in FIGS. 2A-D.

TABLE 1
Recognition of JRFL Env-expressing cells

| | Mean | |
|---|---|---|
| Construct | (normalized RLU) | SEM |
| 17b antibody alone | 0.02 | 0.01 |
| 17b + sCD4 mix | 0.49 | 0.08 |
| 17b-sCD4 conjugate | 0.90 | 0.15 |
| X5 antibody alone | 0.07 | 0.01 |
| X5 + sCD4 mix | 0.58 | 0.06 |
| X5-sCD4 conjugate | 0.65 | 0.07 |
| N5-i5 antibody alone | 0.11 | 0.03 |
| N5-i5 + sCD4 mix | 0.16 | 0.03 |
| N5-i5-sCD4 conjugate | 0.94 | 0.18 |
| A32 antibody alone | 0.07 | 0.02 |
| A32 + sCD4 mix | 0.18 | 0.07 |
| A32-sCD4 conjugate | 1.28 | 0.41 |

None of the four non-neutralizing antibodies tested alone was able to recognize Env-expressing cells (FIGS. 2A-D; Table 1). When mixed with sCD4, the CoRBS specific nnAbs 17b and X5 recognized cell-surface Env, with a normalized RLU of 0.49 and 0.58, respectively (FIGS. 2A and 2B; Table 1), but the Cluster A specific non-neutralizing antibodies N5-i5 and A32 did not, having a normalized RLU of 0.16 and 0.18, respectively (FIGS. 2C and 2D; Table 1). This is consistent with published information on Env conformations post sCD4 binding, where sCD4 is able to trigger the conformation changes required to expose the CoRBS but not the Cluster A region. However, Ab-CD4 conjugates containing the Ab arm of either of the two CD4i non-neutralizing antibody classes CoRBS and Cluster A efficiently recognized Env-expressing cells (FIGS. 2A-D; Table 1). Interestingly, the binding levels of Cluster A Ab-CD4 conjugates (0.94 and 1.28) were comparable to CoRBS Ab-CD4s specificities (0.90 and 0.65), indicating a similar level of epitope exposure. This is of particular interest as the Cluster A region maps to the Env trimer interior and requires significant structural rearrangements of the trimer post CD4-binding for exposure. This also indicates that the requirement of cooperativity between CoRBS and Cluster A non-neutralizing antibodies for efficient recognition of unliganded cell-surface Env pretreated with sCD4 or CD4mc may be overcome with a Cluster A Ab-CD4 conjugate.

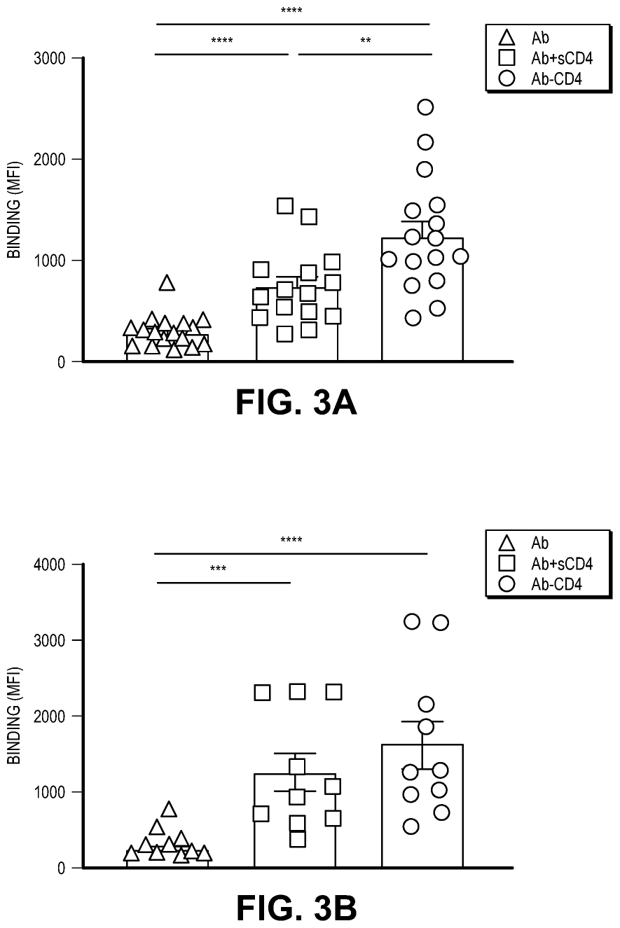

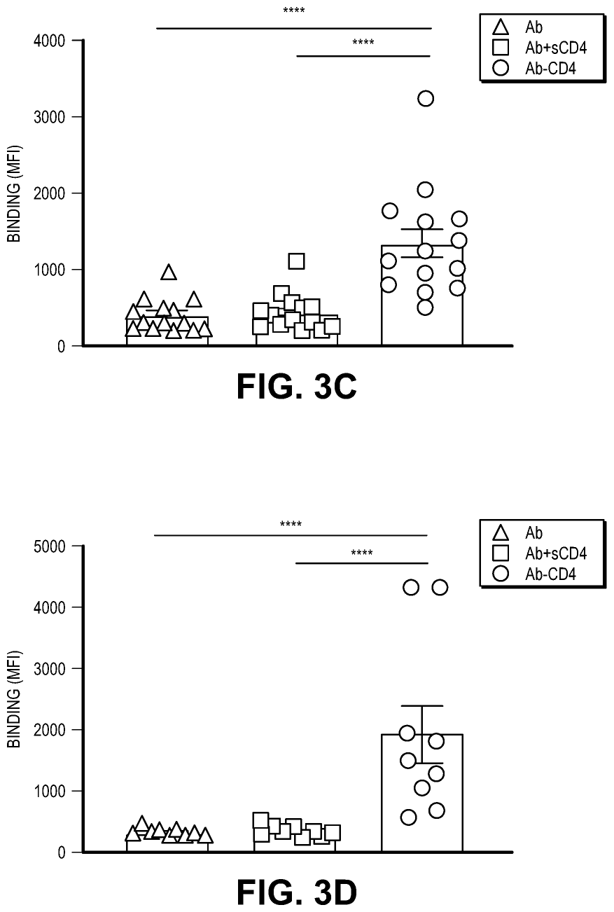

The capacity of the Ab-CD4 conjugates disclosed herein to recognize HIV-1-infected cells was also investigated by flow cytometry. Activated primary CD4+ T cells were infected with the primary HIV-1 isolate JRFL; two days later, the infected cells were incubated with non-neutralizing antibodies alone (10 μg/ml each of 17b, X5, N5-i5, and A32), a mixture of non-neutralizing antibodies and sCD4 (10 μg/ml), or Ab-CD4 conjugates (10 ag/ml prepared as described above. Antibody binding was measured using Alexa fluor-647-conjugated secondary antibodies. Consistent with the protective activities of Nef and Vpu proteins, which limit cell-surface Env-CD4 interaction, infected primary CD4+ T cells were mainly resistant to recognition by antibodies targeting the CoRBS (17b and X5; FIGS. 3A and 3B) or the Cluster A (N5-i5 and A32; FIGS. 3C and 3D) regions when used alone. Addition of sCD4 as a mixture significantly enhanced the capacity of the CoRBS Abs 17b and X5 to recognize HIV-1-infected cells, and both CoRBS Ab-CD4 conjugate molecules (17b-CD4 and X5-CD4) directly recognized infected cells (FIGS. 3A and 3B). The capacity of CoRBS Ab-CD4 conjugates to recognize HIV-1-infected cells was similar or superior to their unconjugated antibody counterparts.

In contrast, Cluster A Abs failed to recognize infected cells either when used alone or in the presence of a mixture together with sCD4 (FIGS. 3C and 3D). Only the Cluster A Ab-CD4 conjugates (N5-i5-CD4 and A32-CD4) efficiently bound to JRFL-infected cells (FIGS. 3C and 3D). The results are shown below in Table 2

TABLE 2
Recognition of JRFL-infected cells

| | Mean | |
|---|---|---|
| Construct | (MFI) | SEM |
| 17b antibody alone | 311.00 | 40.31 |
| 17b + sCD4 mix | 740.20 | 94.81 |
| 17b-sCD4 conjugate | 1250.00 | 142.20 |
| X5 antibody alone | 342.20 | 62.08 |
| X5 + sCD4 mix | 1281.00 | 245.70 |
| X5-sCD4 conjugate | 1649.00 | 309.90 |
| N5-i5 antibody alone | 401.50 | 58.26 |
| N5-i5 + sCD4 mix | 437.40 | 64.40 |
| N5-i5-sCD4 conjugate | 1348.00 | 189.10 |
| A32 antibody alone | 287.70 | 20.46 |
| A32 + sCD4 mix | 337.70 | 32.03 |
| A32-sCD4 conjugate | 1937.00 | 478.10 |

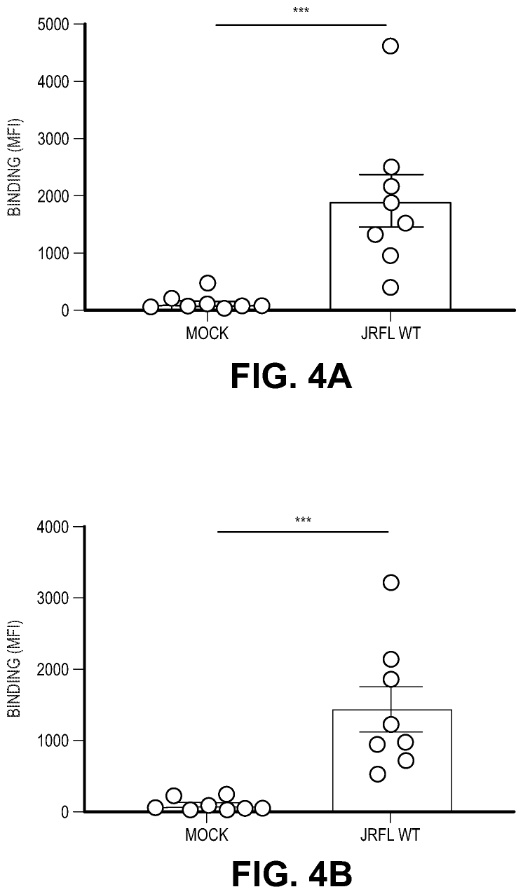

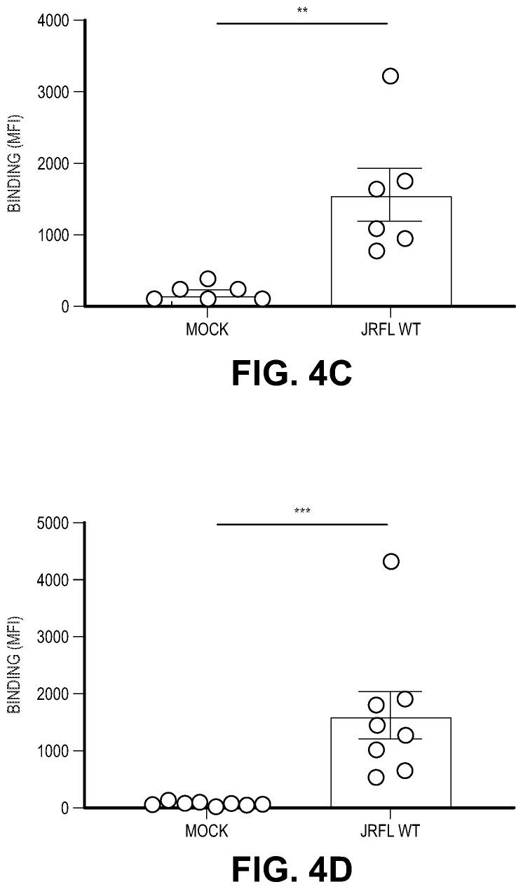

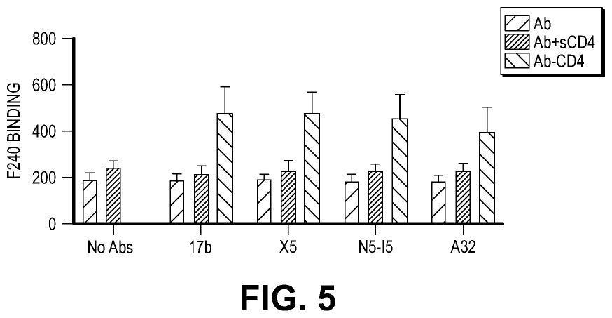

Notably, all four of the developed Ab-CD4 conjugates specifically recognized HIV-1-infected cells over autologous mock-infected cells (FIGS. 4A-D) and triggered Env conformational changes as indicated by increased binding of the anti-gp41 mAbs F240 upon Ab-CD4 interaction (FIG. 5). The graphs shown in FIG. 5 represent the compiled median fluorescence intensities on the infected (p24+) cell population, based on recognition of primary CD4+ T cells infected with HIV-1 JRFL by Alexa-Fluor 647-conjugated F240 mAbs in the presence of Ab-CD4 or Ab (10 μg/ml)+/−sCD4 (10 μg/ml), as evaluated by flow cytometry. Error bars indicate means±SEM for 3 independent experiments. Since F240 targets a gp41 epitope that is only exposed late in the entry process post CD4 binding, similar to CD4i Env epitopes, these data suggest that Ab-CD4 molecules may “push” the Env trimer into similar downstream conformational states. While not wishing to be bound by theory, it is believed that this conformation is likely more open than the conformation present when triggered by sCD4 alone or with a mixture of sCD4 and non-neutralizing antibodies, as indicated by the more efficient F240 epitope exposure for Env treated with Ab-CD4 conjugates as compared to non-neutralizing antibodies, sCD4, or a mixture of sCD4 plus non-neutralizing antibodies (FIG. 5).

### Example 3—Ab-CD4 Conjugates Eliminate HIV-1 Infected Cells Through an ADCC Mechanism

The ADCC activity against HIV-1 infected cells was evaluated using a previously described flow cytometry-based ADCC assay [41] using primary CD4+ T cells infected with JRFL as target cells and autologous PBMCs as effector cells. The ADCC-mediated elimination of infected cells was determined by the loss of p24+target cells upon treatment with effector cells and either Ab-CD4 conjugates or antibodies with or without sCD4. Since Ab-CD4 conjugates showed similar biological activity within each class of non-neutralizing antibodies, one conjugate from each class was tested (17b-CD4 and N5-i5-CD4).

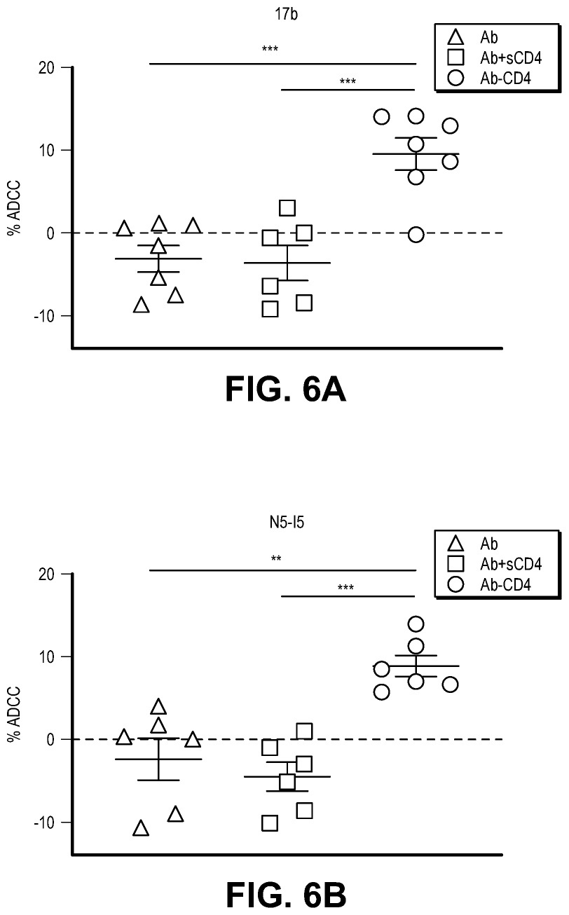

The results are shown below in Table 3 and illustrated in FIGS. 6A-D. JRFL-infected cells were found to be resistant to ADCC mediated by 17b, even in the presence of sCD4 (FIG. 6A). In contrast, the 17b-CD4 conjugate mediated ADCC against JRFL-infected cells (FIG. 6A). The results confirm that effective recognition of infected cells by the 17b-CD4 conjugate translated to ADCC. A similar ADCC killing was observed with the N5-i5-CD4 conjugate molecule, indicating effective triggering of effector cells and ADCC by Ab-CD4 conjugates targeting the Cluster A region (FIG. 6B). These results suggest that both classes of Ab-CD4 conjugates can directly recognize and mediate ADCC of HIV-1-infected cells, unlike mixtures of the 17b or N5-i5 antibody with sCD4.

TABLE 3
ADCC activity against JRFL-infected cells

| | Mean | |
|---|---|---|
| Construct | (% ADCC) | SEM |
| 17b antibody alone | −3.10 | 1.60 |
| 17b + sCD4 mix | −3.65 | 2.09 |
| 17b-sCD4 conjugate | 9.54 | 1.94 |
| N5-i5 antibody alone | −2.42 | 2.49 |
| N5-i5 + sCD4 mix | −4.53 | 1.76 |
| N5-i5-sCD4 conjugate | 8.84 | 1.30 |

### Example 4—Ab-CD4 Conjugates Show Neutralizing Activity Against Tier 1 and Tier 2 HIV-1 Env Trimer

While some antibodies specific for CoRBS are capable of weak neutralization of Tier 1 viruses, the Cluster A antibodies represent a group of canonical non-neutralizing antibodies incapable of impacting virus through neutralization. Because the Ab-CD4 conjugate molecules disclosed herein bound to Env present on infected cells, the ability of the Ab-CD4 conjugates to recognize the trimeric Env present on HIV-1 virions was also investigated. A previously described virus-capture assay was used [35], which measures binding of HIV-1 virions by mAbs immobilized on enzyme-linked immunosorbent assay (ELISA) plates. Viral particles were produced by co-transfecting HEK293T cells with the pNL4.3 Nef Luc Env-construct, a plasmid encoding the tier-2 HIV-1_{JRFL} Env and a plasmid encoding the G glycoprotein from the vesicular stomatitis virus (VSV-G). This generated a virus capable of a single round of infection. Virus containing supernatants were added to plates coated with antibody alone or Ab-CD4 conjugates and unbound virions removed by washing. Antibody-mediated retention of HIV-1 virions was assessed by addition of HEK293T cells. Infection of this CD4-negative cell-line is mediated by VSV-G and measured by luciferase activity two days post-infection.

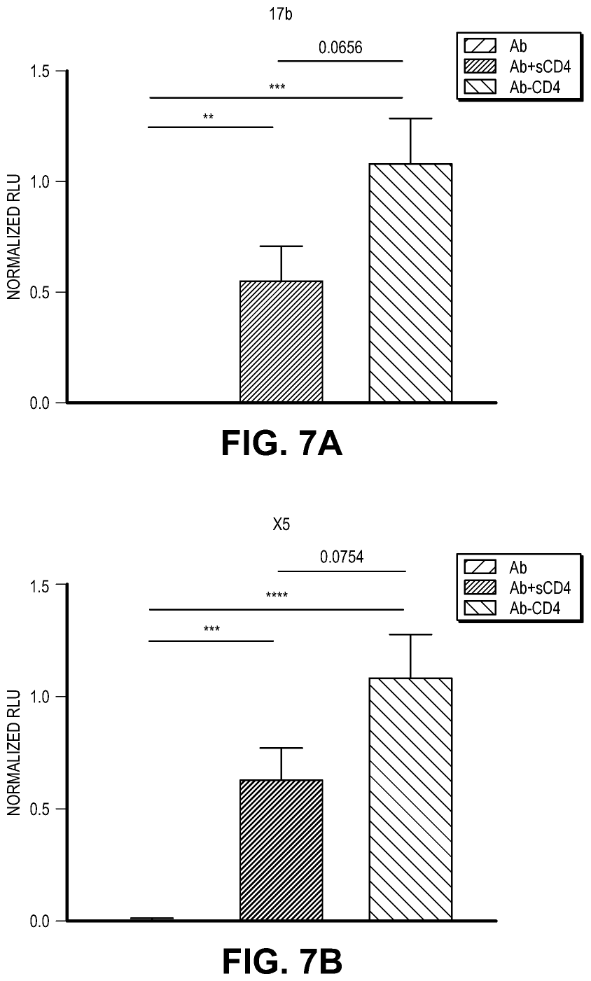

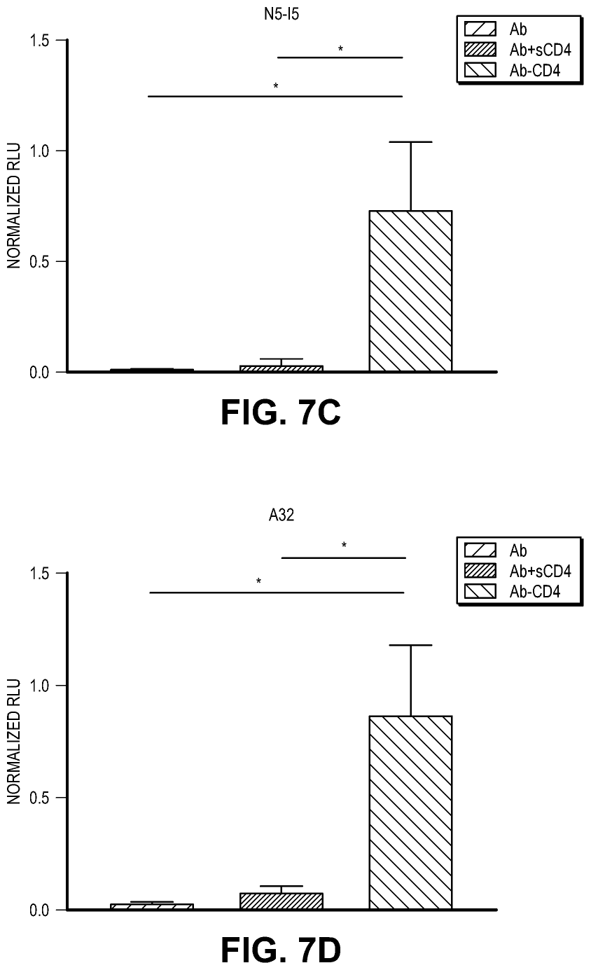

As shown in FIGS. 7A-D and Table 4 below, CoRBS antibodies (17b and X5; FIGS. 7A and 7B) and Cluster A antibodies (N5-i5 and A32, FIGS. 7C and 7D) alone failed to capture HIV-1 virions. Furthermore, consistent with the pattern observed for HIV-1 infected cells, an enhanced capacity for CoRBS antibodies to capture HIV-1 viral particles in the presence of sCD4 was observed, but this was not the case for Cluster A antibodies, which were unable to retain virions either in the presence or absence of sCD4. On the other hand, CoRBS-CD4 conjugates exhibited efficient virion capture (FIGS. 7A and 7B). For Cluster A antibodies, virion capture was observed only when the antibody was directly linked to CD4 (FIGS. 7C and 7D), confirming again that Cluster A-CD4 conjugate molecules are able to trigger conformational rearrangements of the Env trimer required for C1C2 epitope exposure. Notably in all cases, the retention of virions by Ab-CD4 conjugates tended to be superior to their unconjugated counterpart antibodies, either alone or in the presence of sCD4.

TABLE 4
Recognition of JRFL Env present on the virus

| | Mean | |
|---|---|---|
| Construct | (normalized RLU) | SEM |
| 17b antibody alone | 0.0021 | 0.0011 |
| 17b + sCD4 mix | 0.5591 | 0.1597 |
| 17b-sCD4 conjugate | 1.0860 | 0.2010 |
| X5 antibody alone | 0.0017 | 0.0009 |
| X5 + sCD4 mix | 0.6359 | 0.1387 |
| X5-sCD4 conjugate | 1.0920 | 0.1840 |
| N5-i5 antibody alone | 0.0176 | 0.0066 |
| N5-i5 + sCD4 mix | 0.0399 | 0.0166 |
| N5-i5-sCD4 conjugate | 0.7446 | 0.2970 |
| A32 antibody alone | 0.0353 | 0.0059 |
| A32 + sCD4 mix | 0.0748 | 0.0236 |
| A32-sCD4 conjugate | 0.8713 | 0.3121 |

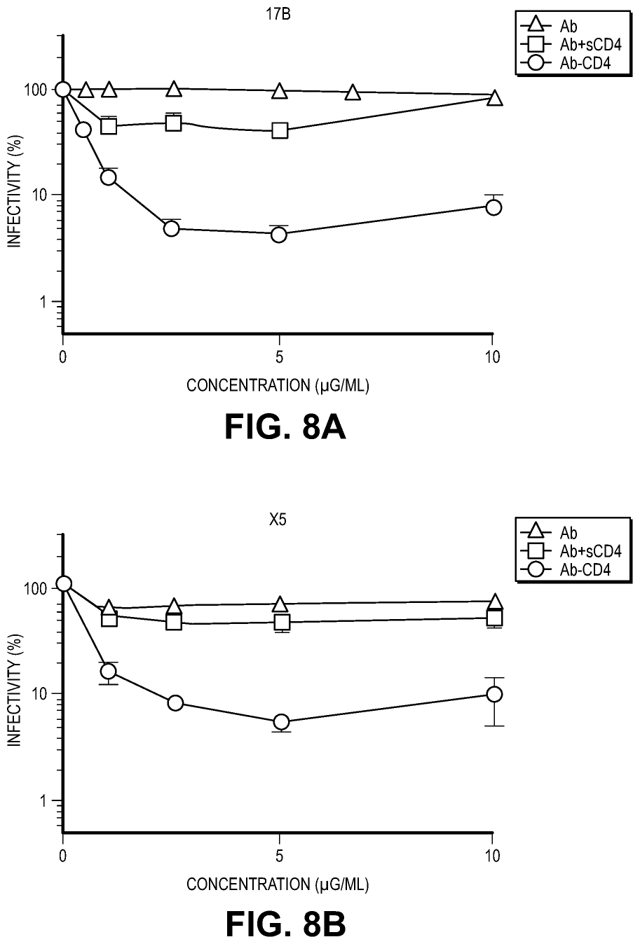

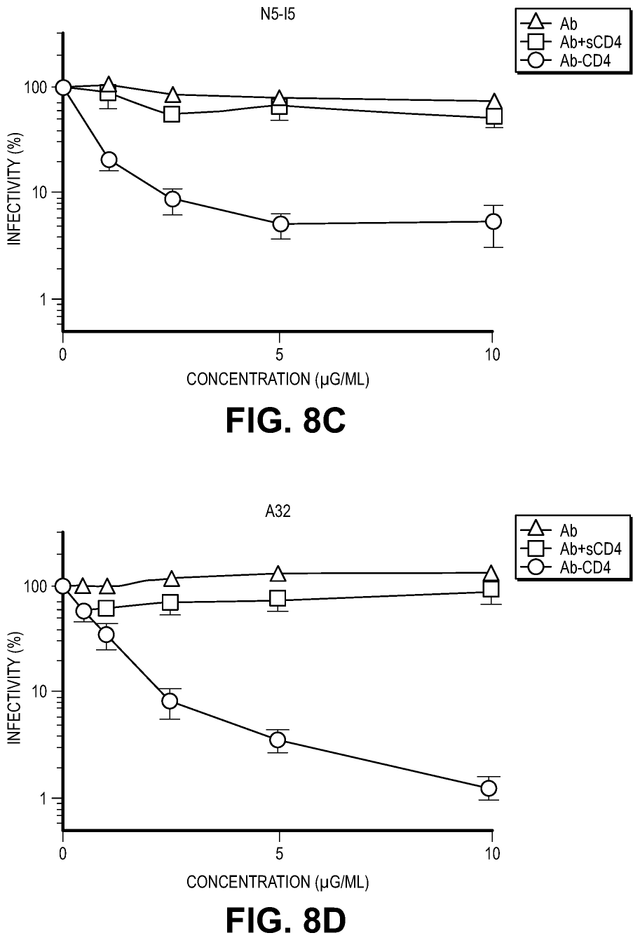

Finally, whether the increased capacity of Ab-CD4 conjugates to capture viral particles translated into direct neutralizing activity was evaluated. While CoRBS (17b or X5; FIGS. 8A and 8B) and Cluster A (N5-i5 or A32; FIGS. 8C and 8D) antibodies alone failed to neutralize pseudoviruses bearing the Env from JRFL, both Ab-CD4 conjugate classes did (FIGS. 8A-D). Neutralization IC50 and IC80 values against JRFL Env isolates for each of the four Ab-CD4 conjugates are shown below in Table 5.

TABLE 5
Neutralization activity against JRFL

| Construct | IC₅₀ (μg/mL) | IC₈₀ (μg/mL) |
|---|---|---|
| 17b antibody alone | >10 | >10 |
| 17b + sCD4 mix | 2.7740 | >10 |
| 17b-sCD4 conjugate | 0.2394 | 0.9596 |
| X5 antibody alone | >10 | >10 |
| X5 + sCD4 mix | 3.2260 | >10 |
| X5-sCD4 conjugate | 0.2025 | 0.8100 |
| N5-i5 antibody alone | >10 | >10 |
| N5-i5 + sCD4 mix | >10 | >10 |
| N5-i5-sCD4 conjugate | 0.2618 | 1.0472 |
| A32 antibody alone | >10 | >10 |
| A32 + sCD4 mix | >10 | >10 |
| A32-sCD4 conjugate | 0.4971 | 1.9884 |

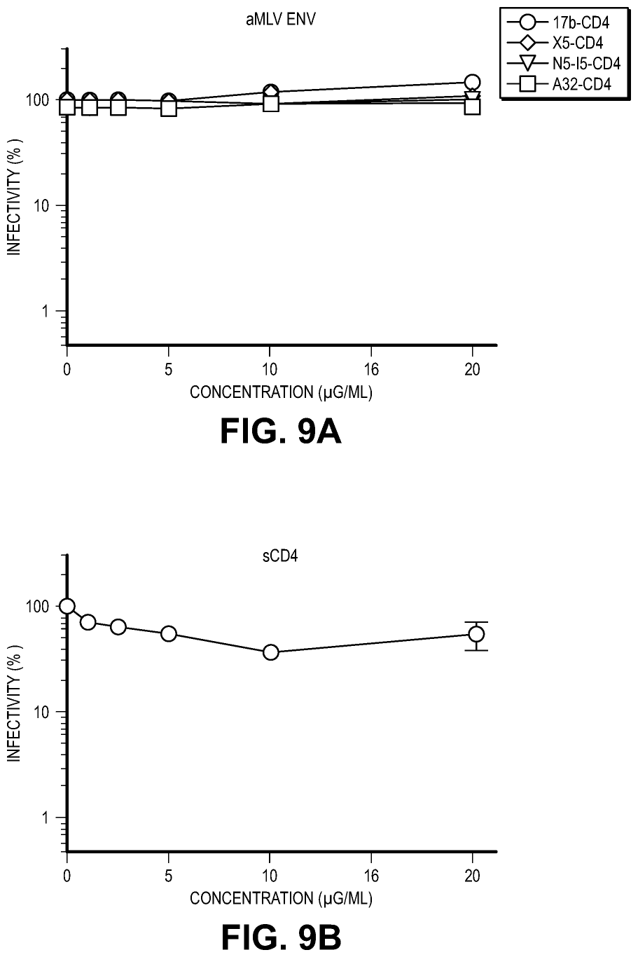

This activity was specific to HIV-1 Env, since no neutralization was observed against pseudoviruses bearing A-MLV Env (FIG. 9A). The neutralizing activity of Ab-CD4 conjugates was superior to sCD4 alone (FIG. 9B) or in combination with the non-neutralizing antibodies (FIG. 8A-D), confirming that these Ab-CD4 conjugate molecules represent an improvement over an unconjugated mixture of both moieties together.

The neutralizing capacity of these molecules was then tested against a panel of virus or pseudoviruses bearing HIV-1 Env from Tier 1 and 2 viral strains. Consistent with the conserved nature of the epitopes targeted by the two classes of Ab-CD4 conjugate molecules, they showed neutralization activities against viruses or pseudoviruses bearing HIV-1 Env from clades A, B, C and CRF01_AE strains, as shown below in Table 6 below, wherein the neutralization half-maximal inhibitory concentrations of the different Ab-CD4 conjugates for each virus are summarized.

TABLE 6
Neutralization IC₅₀ of Ab-CD4 conjugates

| | | Neutralization IC₅₀ (μg/mL) | | | |
|---|---|---|---|---|---|
| Clade | Env | 17b-CD4 | X5-CD4 | N5-i5-CD4 | A32-CD4 |
| Clade B | YU2 | 0.082 | 0.152 | 0.299 | 0.264 |
| | AD8 | 0.877 | 0.840 | 3.274 | 4.263 |
| | JRCSF | 0.807 | 1.968 | 1.469 | 2.992 |
| Clade A | BG505 | 2.637 | 3.168 | 9.603 | 7.894 |
| Clade C | C1086 | 0.269 | 0.473 | 0.431 | 0.865 |
| | ZM109 | 0.113 | 0.285 | 0.161 | 0.469 |
| CRF01_AE | CM244 | 0.609 | 1.377 | 1.048 | 2.176 |

### Example 5—Biological Activity of Ab-CD4 Conjugates Relies on Both Ab and CD4 Interaction with Env Trimer

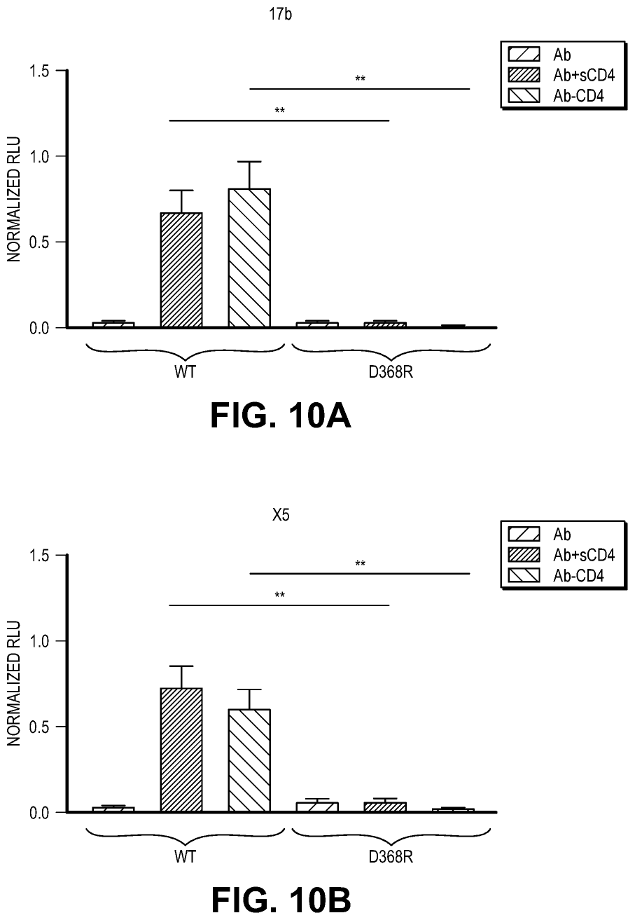

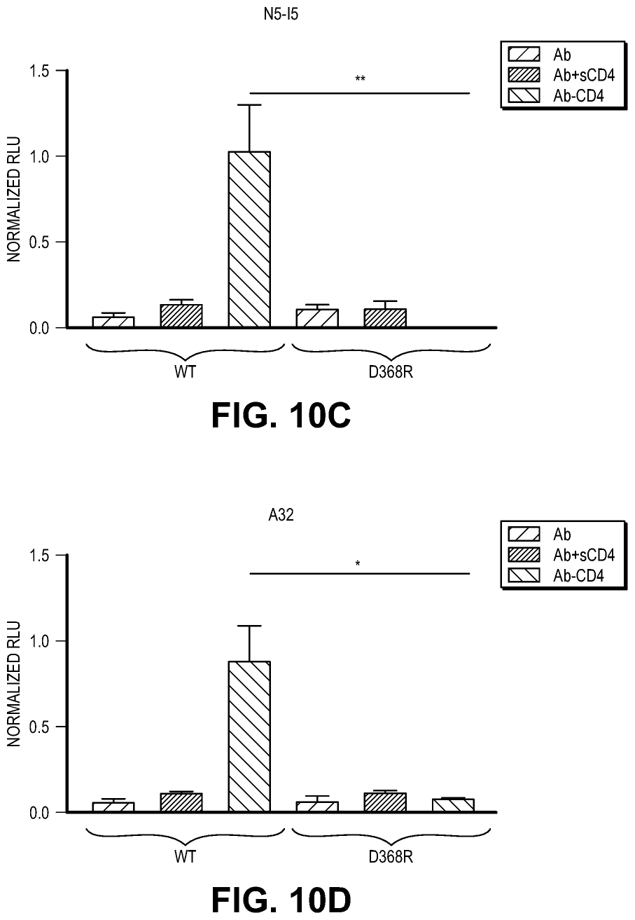

The mechanism of interaction of the Ab-CD4 conjugates with the Env trimer was investigated to determine if both the CD4 and the antibody arms are engaged in binding to Env on HIV-1-infected cells/viral particles and are involved in the ADCC/neutralization activity. To assess the specific role of the CD4 moiety, a D368R mutation was introduced into the CD4 binding site of HIV-1_{JRFL} Env. This mutation was shown to abrogate the Env-CD4 interaction [16, 42, 51, 57]. As shown in FIGS. 10A-D, introduction of this mutation totally inhibited the capacity of both classes of Ab-CD4 conjugates to recognize cell-surface expressed HIV-1_{JRFL} Env. This confirms that the sCD4 moiety of these Ab-CD4 conjugates is required to target the unbound “closed” Env.

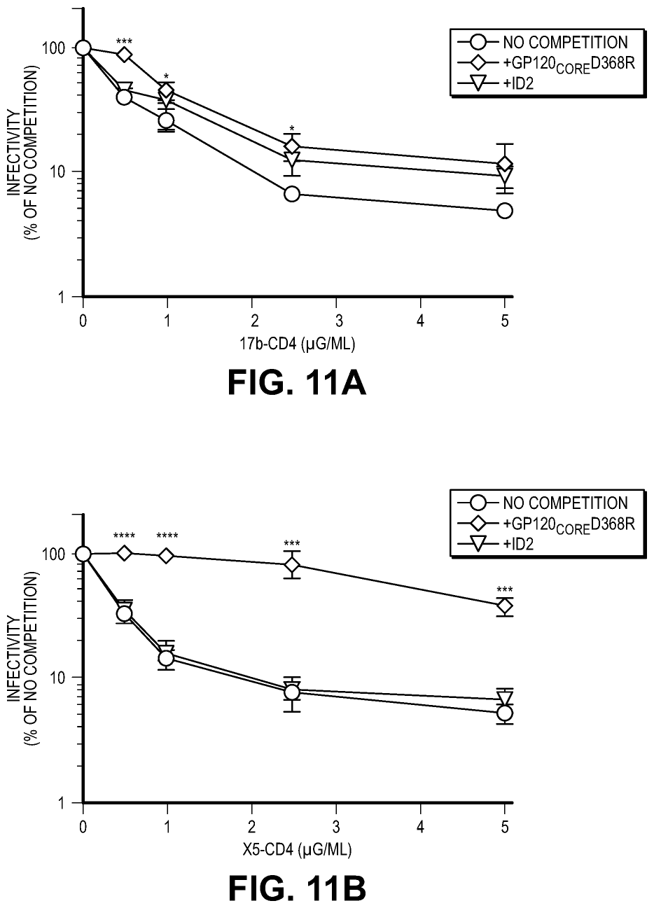

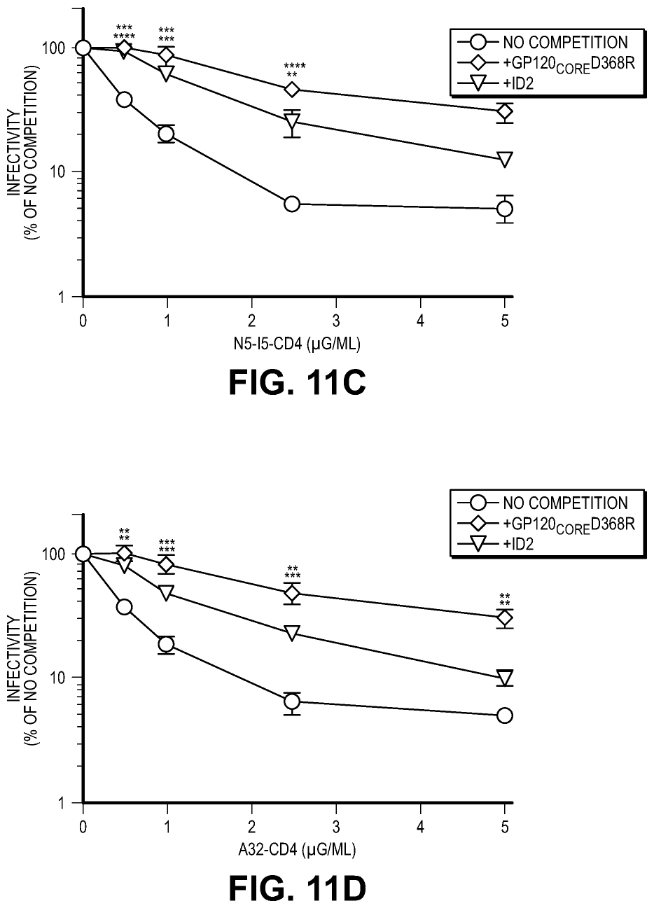

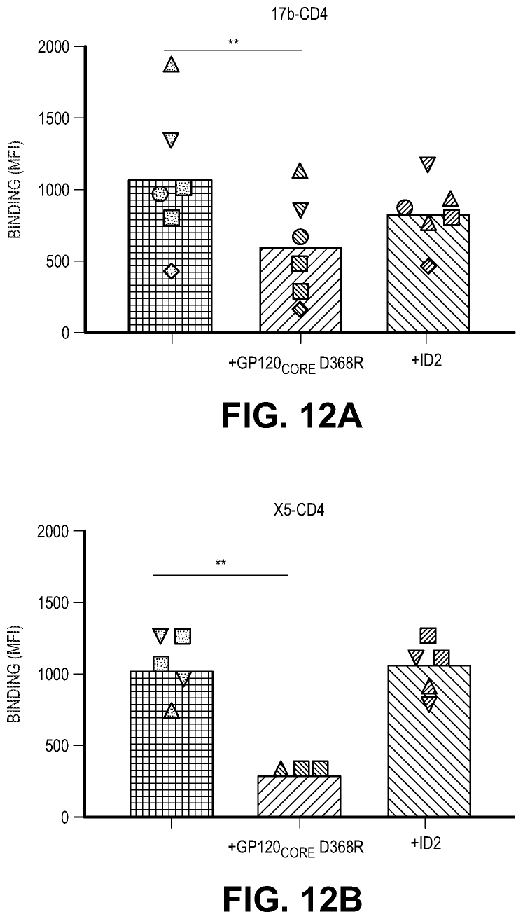

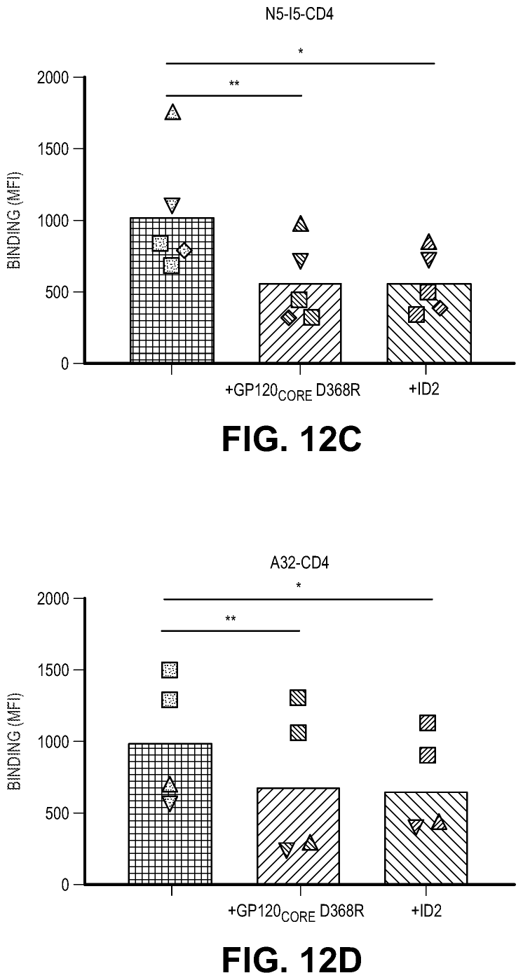

To assess the specific role of the antibody moiety in the biological activities of the Ab-CD4 conjugates, competition experiments were done using two different gp120 probes, gp120_{core} D368R and ID2. First, a CD4-bound stabilized gp120 core protein, lacking variable regions V1, V2, V3, and V5 and harboring the CD4-binding site D368R mutation was used. This recombinant protein can interact with CoRBS and Cluster A antibodies but not with CD4 [58, 59]. Pre-incubation of CoRBS-CD4 conjugates or Cluster A-CD4 conjugates with the gp120_{core} D368R probe significantly reduced their capacity to neutralize HIV-1 and to recognize HIV-1-infected primary CD4+ T cells (FIGS. 11A-D and FIGS. 12A-D, blue line).

Conversely, competition with an inner domain stabilized gp120 probe (ID2), having the ability to interact with Cluster A antibodies but not with CoRBS antibodies or CD4, only affected the biological activities of N5-i5-CD4 (FIGS. 11C and 12C, red line) and A32-CD4 (FIGS. 12C and 12D, red line). This suggests that competing with the antibody moiety significantly reduces the capacity of the Ab-CD4 conjugates to neutralize HIV-1 and to target infected cells. All together, these results suggest that the biological activities mediated by the Ab-CD4 conjugate depend on both antibody and CD4 moiety interaction with HIV-1 Env.

### Example 6—A32-CD4 Conjugates Decrease the Size of the Viral Reservoir in Hu-Mice

For in vivo studies, A32-CD4 hybrids were produced to include GASDALIE (G236A, S239D, A330L, 1332E) mutations with the IgG to enhance binding to Fcγ receptors present on the effector cell surface and LALA (L234A/L235A) mutations to produce Fc-effector-null variants. NK cell depletion experiments in a hu-mice NSG-tgIL15-hu-HSC mice model of HIV-1-infection support ADCC and NK cell functions as reported previously. [72-76].

As described herein, the Cluster A targeting A32-CD4 hybrid was evaluated to determine if it could eliminate infected cells in HIV-1_{JRFL}-infected hu-mice generated by transplanting human PBMC in the NSG-IL15 mouse strain (Hu-PBL model). Hu-PBL mice were infected with HIV-1_{JRCSF} (30,000 pfu, IP). At day 5 after infection with HIV-1_{JRCSF}, when plasma viral loads (PVL) were clearly detectable without a significant depletion of peripheral CD4+ T cells, the mice (n=2 per cohort) were treated once with 1.5 mg of a vehicle control, A32-CD4 tAb, the corresponding LALA variant, or the GASDALIE (FcE) variant, injected subcutaneously. The PVL was measured again on day 10. Biodistribution of A32-CD4 in the blood was measured by ELISA against the gp120 stabilized (ID2) inner domain.

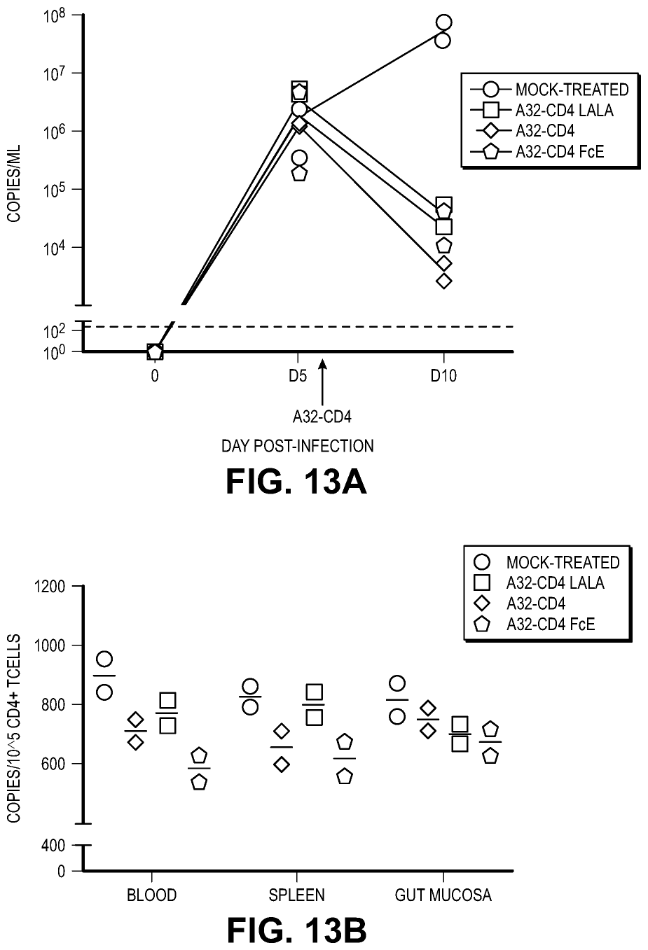

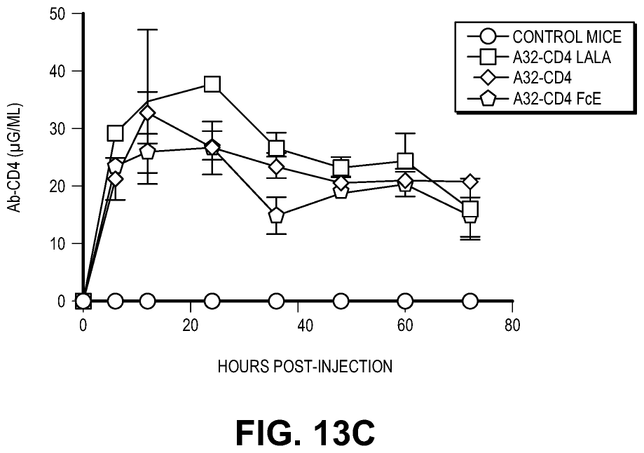

As shown in FIG. 13A, while PVL continued to rise in the mock-treated control mice, treatment with any of the A32-CD4 variants resulted in control of PVL at day 10 post-infection, demonstrating a strong neutralization effect of the Ab conjugates (FIG. 13A). The same groups also showed reductions in integrated HIV-1 proviral DNA copies in CD4+ T cells immunopurified from gut mucosa, peripheral blood and from the spleen, which harbor in vivo viral reservoirs, as shown in FIG. 13B. This suggests a direct impact on the HIV viral reservoir. Moreover, vDNA levels in mice treated with the LALA tAb variant tended to be higher than the levels observed with the wild-type A32-CD4 tAb, and with the tAb FcE variant, the levels observed were lower than those of the wild-type A32-CD4 tAb, as shown in FIGS. 13B and 13C. Taken together, these data suggest that Fc-effector mechanisms were involved in the reduction of tissue viral reservoirs in Hu-PBL mice.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### REFERENCES

1. Kowalski M, et al. Functional regions of the envelope glycoprotein of human immunodeficiency virus type 1. Science 237, 1351-1355 (1987).
2. Alkhatib G, et al. CC CKR5: a RANTES, MIP-1alpha, MIP-1beta receptor as a fusion cofactor for macrophage-tropic HIV-1. Science 272, 1955-1958 (1996).
3. Choe H, et al. The beta-chemokine receptors CCR3 and CCR5 facilitate infection by primary HIV-1 isolates. Cell 85, 1135-1148 (1996).
4. Deng H, et al. Identification of a major co-receptor for primary isolates of HIV-1. Nature 381, 661-666 (1996).
5. Doranz B J, et al. A dual-tropic primary HIV-1 isolate that uses fusin and the beta-chemokine receptors CKR-5, CKR-3, and CKR-2b as fusion cofactors. Cell 85, 1149-1158 (1996).
6. Dragic T, et al. HIV-1 entry into CD4+ cells is mediated by the chemokine receptor C C-CKR-5. Nature 381, 667-673 (1996).
7. Feng Y, et al. HIV-1 entry cofactor: functional cDNA cloning of a seven-transmembrane, G protein-coupled receptor. Science 272, 872-877 (1996).
8. Trkola A, et al. CD4-dependent, antibody-sensitive interactions between HIV-1 and its co-receptor CCR-5. Nature 384, 184-187 (1996).
9. Wu L, et al. CD4-induced interaction of primary HIV-1 gp120 glycoproteins with the chemokine receptor CCR-5. Nature 384, 179-183 (1996).
10. Lu M, et al. A trimeric structural domain of the HIV-1 transmembrane glycoprotein. Nat Struct Biol 2, 1075-1082 (1995).
11. Weissenhorn W, et al. Atomic structure of the ectodomain from HIV-1 gp41. Nature 387, 426-430 (1997).
12. Tolbert W D, et al. Structural Basis for Epitopes in the gp120 Cluster A Region that Invokes Potent Effector Cell Activity. Viruses 11, (2019).
13. Lewis G K, et al. Conformational Masking and Receptor-Dependent Unmasking of Highly Conserved Env Epitopes Recognized by Non-Neutralizing Antibodies That Mediate Potent ADCC against HIV-1. Viruses 7, 5115-5132 (2015).
14. Munro J B, et al. Conformational dynamics of single HIV-1 envelope trimers on the surface of native virions. Science 346, 759-763 (2014).
15. Tolbert W D, et al. Targeting the Late Stage of HIV-1 Entry for Antibody-Dependent Cellular Cytotoxicity: Structural Basis for Env Epitopes in the C1I Region. Structure 25, 1719-1731 e1714 (2017).
16. Veillette M, et al. The HIV-1 gp120 CD4-Bound Conformation Is Preferentially Targeted by Antibody-Dependent Cellular Cytotoxicity-Mediating Antibodies in Sera from HIV-1-Infected Individuals. Journal of virology 89, 545-551 (2015).
17. Gohain N, et al. Cocrystal Structures of Antibody N60-i3 and Antibody JR4 in Complex with gp120 Define More Cluster A Epitopes Involved in Effective Antibody-Dependent Effector Function against HIV-1. Journal of virology 89, 8840-8854 (2015).
18. Veillette M, et al. Interaction with cellular CD4 exposes HIV-1 envelope epitopes targeted by antibody-dependent cell-mediated cytotoxicity. Journal of virology 88, 2633-2644 (2014).
19. Acharya P, et al. Structural definition of an antibody-dependent cellular cytotoxicity response implicated in reduced risk for HIV-1 infection. Journal of virology 88, 12895-12906 (2014).
20. Guan Y, et al. Diverse specificity and effector function among human antibodies to HIV-1 envelope glycoprotein epitopes exposed by CD4 binding. Proceedings of the National Academy of Sciences of the United States of America 110, E69-78 (2013).
21. Ferrari G, et al. An HIV-1 gp120 envelope human monoclonal antibody that recognizes a C1 conformational epitope mediates potent antibody-dependent cellular cytotoxicity (ADCC) activity and defines a common ADCC epitope in human HIV-1 serum. Journal of virology 85, 7029-7036 (2011).
22. Alsahafi N, et al. An Asymmetric Opening of HIV-1 Envelope Mediates Antibody-Dependent Cellular Cytotoxicity. Cell Host Microbe 25, 578-587 e575 (2019).
23. Finzi A, et al. Topological layers in the HIV-1 gp120 inner domain regulate gp41 interaction and CD4-triggered conformational transitions. Mol Cell 37, 656-667 (2010).
24. Richard J, et al. Co-receptor Binding Site Antibodies Enable CD4-Mimetics to Expose Conserved Anti-cluster A ADCC Epitopes on HIV-1 Envelope Glycoproteins. EBioMedicine 12, 208-218 (2016).
25. Ding S, et al. A Highly Conserved Residue of the HIV-1 gp120 Inner Domain Is Important for Antibody-Dependent Cellular Cytotoxicity Responses Mediated by Anti-cluster A Antibodies. Journal of virology 90, 2127-2134 (2016).
26. Decker J M, et al. Antigenic conservation and immunogenicity of the HIV coreceptor binding site. J Exp Med 201, 1407-1419 (2005).
27. Prevost J, et al. Envelope glycoproteins sampling states 2/3 are susceptible to ADCC by sera from HIV-1-infected individuals. Virology 515, 38-45 (2018).
28. Richard J, et al. Uninfected Bystander Cells Impact the Measurement of HIV-Specific Antibody-Dependent Cellular Cytotoxicity Responses. mBio 9, (2018).
29. Alsahafi N, et al. Nef Proteins from HIV-1 Elite Controllers Are Inefficient at Preventing Antibody-Dependent Cellular Cytotoxicity. Journal of virology 90, 2993-3002 (2015).
30. Anand S P, et al. Two Families of Env Antibodies Efficiently Engage Fc-Gamma Receptors and Eliminate HIV-1-Infected Cells. Journal of virology 93, (2019).
31. von Bredow B, et al. Envelope Glycoprotein Internalization Protects Human and Simian Immunodeficiency Virus-Infected Cells from Antibody-Dependent Cell-Mediated Cytotoxicity. Journal of virology 89, 10648-10655 (2015).
32. Anand S P, et al. Antibody-Induced Internalization of HIV-1 Env Proteins Limits Surface Expression of the Closed Conformation of Env. Journal of virology 93, (2019).
33. Arias J F, et al. Tetherin antagonism by Vpu protects HIV-infected cells from antibody-dependent cell-mediated cytotoxicity. Proceedings of the National Academy of Sciences of the United States of America 111, 6425-6430 (2014).
34. Alvarez R A, et al. HIV-1 Vpu antagonism of tetherin inhibits antibody-dependent cellular cytotoxic responses by natural killer cells. Journal of virology 88, 6031-6046 (2014).
35. Ding S, et al. CD4 Incorporation into HIV-1 Viral Particles Exposes Envelope Epitopes Recognized by CD4-Induced Antibodies. Journal of virology 93, (2019).
36. Laumaea A, Smith A B, 3rd, Sodroski J, Finzi A. Opening the HIV envelope: potential of CD4 mimics as multifunctional HIV entry inhibitors. Curr Opin HIV AIDS 15, 300-308 (2020).
37. Madani N, et al. CD4-mimetic small molecules sensitize human immunodeficiency virus to vaccine-elicited antibodies. Journal of virology 88, 6542-6555 (2014).
38. Lagenaur L A, et al. sCD4-17b bifunctional protein: extremely broad and potent neutralization of HIV-1 Env pseudotyped viruses from genetically diverse primary isolates. Retrovirology 7, 11 (2010).
39. Dey B, et al. Neutralization of human immunodeficiency virus type 1 by sCD4-17b, a single-chain chimeric protein, based on sequential interaction of gp120 with CD4 and coreceptor. Journal of virology 77, 2859-2865 (2003).
40. Gardner M R, et al. AAV-expressed eCD4-Ig provides durable protection from multiple SHIV challenges. Nature 519, 87-91 (2015).
41. Richard J, et al. CD4 mimetics sensitize HIV-1-infected cells to ADCC. Proceedings of the National Academy of Sciences of the United States of America 112, E2687-2694 (2015).
42. Vezina D, et al. Stabilizing the HIV-1 envelope glycoprotein State 2A conformation. Journal of virology, (2020).
43. Tolbert W D, et al. Paring Down HIV Env: Design and Crystal Structure of a Stabilized Inner Domain of HIV-1 gp120 Displaying a Major ADCC Target of the A32 Region. Structure 24, 697-709 (2016).
44. Thali M, et al. Characterization of conserved human immunodeficiency virus type 1 gp120 neutralization epitopes exposed upon gp120-CD4 binding. Journal of virology 67, 3978-3988 (1993).
45. Kwong P D, et al. Structure of an HIV gp120 envelope glycoprotein in complex with the CD4 receptor and a neutralizing human antibody. Nature 393, 648-659 (1998).
46. Darbha R, et al. Crystal structure of the broadly cross-reactive HIV-1-neutralizing Fab X5 and fine mapping of its epitope. Biochemistry 43, 1410-1417 (2004).
47. Moulard M, et al. Broadly cross-reactive HIV-1-neutralizing human monoclonal Fab selected for binding to gp120-CD4-CCR5 complexes. Proceedings of the National Academy of Sciences of the United States of America 99, 6913-6918 (2002).
48. Veillette M, et al. Conformational evaluation ofHIV-1 trimeric envelope glycoproteins using a cell-based ELISA assay. Journal of visualized experiments: JoVE, 51995 (2014).
49. Ding S, et al. A New Family of Small-Molecule CD4-Mimetic Compounds Contacts Highly Conserved Aspartic Acid 368 of HIV-1 gp120 and Mediates Antibody-Dependent Cellular Cytotoxicity. Journal of virology 93, (2019).
50. Mengistu M, et al. Antigenic properties of the human immunodeficiency virus envelope glycoprotein gp120 on virions bound to target cells. PLoS Pathog 11, e1004772 (2015).
51. Richard J, et al. Small CD4 Mimetics Prevent HIV-1 Uninfected Bystander CD4+ T Cell Killing Mediated by Antibody-dependent Cell-mediated Cytotoxicity. EBioMedicine 3, 122-134 (2016).
52. Gohain N, et al. Molecular basis for epitope recognition by non-neutralizing anti-gp41 antibody F240. Sci Rep 6, 36685 (2016).
53. Richard J, et al. Impact of HIV-1 Envelope Conformation on ADCC Responses. Trends Microbiol 26, 253-265 (2018).
54. Scheid J F, et al. Broad diversity of neutralizing antibodies isolated from memory B cells in HIV-infected individuals. Nature 458, 636-640 (2009).
55. Xiang S H, et al. Epitope mapping and characterization of a novel CD4-induced human monoclonal antibody capable of neutralizing primary HIV-1 strains. Virology 315, 124-134 (2003).
56. Xiang S H, et al. Characterization of CD4-induced epitopes on the HIV type 1 gp120 envelope glycoprotein recognized by neutralizing human monoclonal antibodies. AIDS Res Hum Retroviruses 18, 1207-1217 (2002).
57. Richard J, et al. Flow cytometry-based assay to study HIV-1 gp120 specific antibody-dependent cellular cytotoxicity responses. Journal of virological methods 208, 107-114 (2014).
58. Coutu M, Finzi A. HIV-1 gp120 dimers decrease the overall affinity of gp120 preparations for CD4-induced ligands. Journal of virological methods 215-216, 37-44 (2015).
59. Levast B, et al. HIV-1 gp120 envelope glycoprotein determinants for cytokine burst in human monocytes. PLoS One 12, e0174550 (2017).
60. Tolbert W D, et al. Defining rules governing recognition and Fc-mediated effector functions to the HIV-1 co-receptor binding site. BMC Biol 18, 91 (2020).
61. Huston J S, et al. Protein engineering of antibody binding sites: recovery of specific activity in an anti-digoxin single-chain Fv analogue produced in Escherichia coli. Proceedings of the National Academy of Sciences of the United States of America 85, 5879-5883 (1988).
62. Sanders R W, et al. A next-generation cleaved, soluble HIV-1 Env trimer, BG505 SOSIP.664 gp140, expresses multiple epitopes for broadly neutralizing but not non-neutralizing antibodies. PLoS Pathog 9, e1003618 (2013).
63. O'Brien W A, et al. HIV-1 tropism for mononuclear phagocytes can be determined by regions of gp120 outside the CD4-binding domain. Nature 348, 69-73 (1990).
64. Theodore T S, Englund G, Buckler-White A, Buckler C E, Martin M A, Peden K W. Construction and characterization of a stable full-length macrophage-tropic HIV type 1 molecular clone that directs the production of high titers of progeny virions. AIDS Res Hum Retroviruses 12, 191-194 (1996).
65. Medjahed H, Pacheco B, Desormeaux A, Sodroski J, Finzi A. The HIV-1 gp120 major variable regions modulate cold inactivation. Journal of virology 87, 4103-4111 (2013).
66. Montefiori D C, et al. Magnitude and breadth of the neutralizing antibody response in the RV144 and Vax003 HIV-1 vaccine efficacy trials. The Journal of infectious diseases 206, 431-441 (2012).
67. Prevost J, et al. The HIV-1 Env gp120 Inner Domain Shapes the Phe43 Cavity and the CD4 Binding Site. mBio 11, (2020).
68. Lodge R, Lalonde J P, Lemay G, Cohen E A. The membrane-proximal intracytoplasmic tyrosine residue of HIV-1 envelope glycoprotein is critical for basolateral targeting of viral budding in MDCK cells. EMBO J 16, 695-705 (1997).
69. Beaudoin-Bussieres G, et al. Elicitation of Cluster A and Co-Receptor Binding Site Antibodies are Required to Eliminate HIV-1 Infected Cells. Microorganisms 8, (2020).
70. Melillo, B, et al., Small-Molecule CD4-Mimics: Structure-Based Optimization of HIV-1 Entry Inhibition, ACS Med Chem Lett. 7(3): 330-334 (2016).
71. Van Herrewege, Y, et al., CD4 mimetic miniproteins: potent anti-HIV compounds with promising activity as microbicides, J Antimicrob. Chemother. 61(4), 818-26 (2008).
72. A. A. Ahmed, et al., Structural characterization of GASDALIE Fc bound to the activating Fc receptor FcgammaRIIIa, J Struct Biol 194 (2016) 78-89.
73. S. Bournazos, et al., Broadly neutralizing anti-HIV-1 antibodies require Fc effector functions for in vivo activity, Cell 158 (2014) 1243-1253.
74. P. Smith, et al., Mouse model recapitulating human Fcgamma receptor structural and functional diversity, Proc Natl Acad Sci USA 109 (2012) 6181-6.
75. K. O. Saunders, Conceptual Approaches to Modulating Antibody Effector Functions and Circulation Half-Life, Front Immunol 10 (2019) 1296.
76. J. K. Rajashekar, et al., Modulating HIV-1 envelope glycoprotein conformation to decrease the HIV-1 reservoir, Cell Host Microbe 29 (2021) 904-916 e6.

1. An Ab-CD4 conjugate comprising an antibody, at least one linker, and at least one CD4 compound, wherein the antibody binds to a Cluster A region or a co-receptor binding site (CoRBS) of the HIV envelope glycoprotein and comprises an Fc region, wherein the at least one linker links the antibody to the at least one CD4 compound, and wherein the Ab-CD4 conjugate is capable of neutralizing an HIV virus.

2. The Ab-CD4 conjugate of claim 1, wherein the antibody is a full-length antibody.

3. The Ab-CD4 conjugate of claim 1, wherein the Ab-CD4 conjugate comprises two linkers and two CD4 compounds, wherein a first linker links the antibody to a first CD4 compound and a second linker links the antibody to a second CD4 compound.

4. The Ab-CD4 conjugate according to claim 1, wherein the linker is (G₄Xaa)ₙ, wherein Xaa is serine or threonine and n is 2-16 (SEQ ID NO: 71).

5. The Ab-CD4 conjugate according to claim 1, wherein the at least one linker is 40-50 amino acids in length.

6. The Ab-CD4 conjugate according to claim 1, wherein the at least one linker is selected from the group consisting of (G₄S)₆-(G₄T)₂ (SEQ ID NO: 72) and (G₄S)₈ (SEQ ID NO: 73).

7. The Ab-CD4 conjugate according to claim 1, wherein the at least one linker is (PEG)ₙ wherein n is 4-100.

8. The Ab-CD4 conjugate according to claim 1, wherein the linker has a length ranging from about 50 Å to about 200 Å.

9. The Ab-CD4 conjugate according to claim 1, wherein the at least one CD4 compound is linked to the at least one linker in a region of the antibody selected from the group consisting of a C_{L} region, a C_{H}1 region, a C_{H}2 region, and a C_{H}3 region.

10. The Ab-CD4 conjugate according to claim 1, wherein the at least one CD4 compound is selected from the group consisting of soluble CD4 (sCD4) and CD4 mimetic compounds (CD4mc).

11. The Ab-CD4 conjugate according to claim 10, wherein the sCD4 compound consists of domain 1 and domain 2 of sCD4.

12. The Ab-CD4 conjugate according to claim 1, wherein the Ab-CD4 conjugate neutralizes HIV virions.

13. The Ab-CD4 conjugate according to claim 1, wherein the antibody binds to the Cluster A region and is selected from the group consisting of 2.2c, A32, C11, CH20, CH29, CH38, CH40, CH49, CH51, CH52, CH53, CH54, CH55, CH57, CH77, CH78, CH80, CH81, CH89, CH90, CH91, CH92, CH94, DH677.3, JR4, N12-i3, N5-i5, and N60-i3.

14. The Ab-CD4 conjugate according to claim 1, wherein the antibody binds to the CoRBS region and is selected from the group consisting of 17b, 412d, 48d, E51, N12-i2, and X5.

15. A vector comprising a nucleic acid molecule encoding the Ab-CD4 conjugate of claim 1.

16. An isolated host cell comprising the vector of claim 15.

17. A method of killing HIV-infected cells through an Fc-mediated effector function comprising contacting the HIV-infected cells with the Ab-CD4 conjugate of claim 1 in the presence of immune cells that bind to the Fc region of the antibody and mediate the Fc-mediated effector function.

18. The method of claim 17, wherein the Fc-mediated effector function is antibody-dependent cellular cytotoxicity.

19. A method of treating or preventing HIV infection in a subject comprising administering to the subject an effective amount of a pharmaceutical composition comprising the Ab-CD4 conjugate of claim 1.

20. The method according to claim 19, wherein the antibody binds to the Cluster A region and further comprising administering a second Ab-CD4 conjugate comprising a second antibody that binds to the CoRBS.

21. The method according to claim 20, wherein the at least one CD4 compound is an sCD4 compound consisting of domain 1 and domain 2 of sCD4.

22. The method according to claim 19, further comprising administering an anti-retroviral therapy.

23. The method according to claim 22, wherein the anti-retroviral therapy is selected from the group consisting of nucleoside analog reverse-transcriptase inhibitors, nucleotide reverse transcriptase inhibitors, non-nucleoside reverse transcriptase inhibitors, protease inhibitors, entry or fusion inhibitors, maturation inhibitors, and natural antivirals.

24. A pharmaceutical composition comprising:
the Ab-CD4 conjugate of claim 1; and
a pharmaceutically acceptable carrier.

25. The pharmaceutical composition according to claim 24, wherein the antibody binds to the Cluster A region, and wherein the pharmaceutical composition further comprises a second Ab-CD4 conjugate comprising a second antibody that binds to the CoRBS region.
